(19) United States
(12) Patent Application Publication
(10) Pub. No.: US 2014/0023683 A1
(43) Pub. Date: Jan. 23, 2014

(54) IDENTIFICATION OF TRANSMITTED HEPATITIS C VIRUS (HCV) GENOMES BY SINGLE GENOME AMPLIFICATION

(71) Applicants: George M. Shaw, Birmingham, AL (US); Hui Li, Birmingham, AL (US); Beatrice H. Hahn, Birmingham, AL (US); Barton F. Haynes, Durham, NC (US)

(72) Inventors: George M. Shaw, Birmingham, AL (US); Hui Li, Birmingham, AL (US); Beatrice H. Hahn, Birmingham, AL (US); Barton F. Haynes, Durham, NC (US)

(73) Assignees: THE UAB RESEARCH FOUNDATION, Birmingham, AL (US); DUKE UNIVERSITY, Durham, NC (US)

(21) Appl. No.: 13/821,886

(22) PCT Filed: Sep. 8, 2011
(86) PCT No.: PCT/US11/50807

§ 371 Date: Oct. 9, 2013

Related U.S. Application Data
(60) Provisional application No. 61/380,997, filed on Sep. 8, 2010.

(51) Int. Cl. C12Q 1/70; A61K 39/29

(52) U.S. Cl. CPC C12Q 1/707; A61K 39/29
USPC 424/228.1; 435/5; 536/23.72

(57) ABSTRACT

This invention provides methods for identifying HCV genomes and more specifically, methods for identifying nucleotide sequence of viral structural proteins at the time of HCV viral transmission. The method of the invention utilizes single genome amplification and sequencing of circulating virus as well as phylogenetic analysis of the resulting nucleotide sequence for identifying transmitted HCV genomes. Also provided are HCV genomes and corresponding nucleotide sequence for transmitted and circulating HCV virus. The invention further provides methods of administering a vaccine comprising one or more identified transmitted HCV sequences.

### FIELD OF THE INVENTION

The invention provides methods for identifying hepatitis C virus (HCV) genomes that mediate viral transmission and clinical infection. More particularly, the invention provides nucleotide sequences corresponding to specific transmitted HCV genomes, including structural gene nucleotide sequences of global HCV genotypes, subtypes, and drug resistant variants that actively mediate viral infection. The invention further provides methods of administering a vaccine comprising one or more identified HCV sequences.

### BACKGROUND OF THE INVENTION

HCV transmission in humans results most commonly from percutaneous inoculations or exposures at mucosal surfaces (Lindenbach, B. D. et al., 2005, Science 309:623-626; Moradpour, D. et al., 2007, Nat Rev Microbiol 5:453-463; Ray, S. C. et al., 2010, Mandell, Douglas, and Bennett's Principles and Practice of Infectious Diseases, Churchill Livingstone Elsevier, Philadelphia, vol, 2:2157-2185; Strickland, G. T. et al., 2008, Lancet Infect Dis 8:379-386; Tohme, R. A. et al., 2010, Hepatology (pre-print Epub:DOI:10.1002/hep.23808)). Experimentally, it has not been possible to identify and characterize by direct analytical methods HCV at or near the moment of transmission, yet it is this virus that antibody or cell-based vaccines, antiviral drugs, and the natural immune responses should interdict (Strickland, G. T. et al., 2008, Lancet Infect Dis 8:379-386; Bowen, D. G. et al., 2005, Nature 436:946-952; Cox, A. L. et al., 2005, J Exp Med 201:1741-1752; Kuntzen, T. et al., 2007, J Virol 81:11658-11668; Liu, C. H. et al., 2006, Clin Infect Dis 42:1254-1259; Liu, L. et al., 2010, J Virol 84:5067-5077). An important step in achieving a molecular understanding of HCV transmission and for improved development of effective HCV vaccines and drugs is an accurate and precise description of the transmitted virus (or viruses) and sequences evolving from them during the critical period leading to productive clinical infection and thereafter.

Previous reports examined the molecular basis of HCV transmission by analyzing the genetic composition of viruses sampled between one and six months following infection (Strickland, G. T. et al., 2008, Lancet Infect Dis 8:379-386; Bowen, D. G. et al., 2005, Nature 436:946-952; Cox, A. L. et al., 2005, J Exp Med 201:1741-1752; Kuntzen, T. et al., 2007, J Virol 81:11658-11668; Liu, C. H. et al., 2006, Clin Infect Dis 42:1254-1259; Liu, L. et al., 2010, J Virol 84:5067-5077). A common methodological approach in these studies was to derive viral sequences from the plasma of patients by bulk or near-limiting dilution polymerase chain reaction (PCR) amplification of viral nucleic acid (viral RNA, vRNA), followed by cloning, sequencing, and phylogenetic analysis. Alternatively, bulk amplified viral nucleic acids were analyzed by 454 deep sequencing where only a small fraction of the gene of interest is interrogated (Wang, G. P. et al., 2010, J Virol 84:6218-6228). While these approaches provide an approximation of the complexity of virus populations in acute and early infection, they have significant limitations. 454 deep sequencing, for example, provides sequence information for only short regions of the genome and has a significant polymerase error rate, and as a consequence allows for only qualitative inferences regarding the genetic identity and complexity of virus populations. Bulk or near-endpoint PCR followed by cloning and sequencing is compromised by the introduction of Taq polymerase errors (Palmer, S. et al., 2005, J Clin Microbiol 43:406-413; Salazar-Gonzalez, J. F. et al., 2008, J Virol 82:3952-3970; Simmonds, P. et al., 1990, J Virol 64:5840-5850), Taq polymerase-mediated template switching (recombination) (Salazar-Gonzalez, J. F. et al., 2008, J Virol 82:3952-3970; Meyerhans, A. et al., 1990, Nucleic Acids Res 18:1687-1691; Shriner, D. et al., 2004, Genetics 167:1573-1583), and non-proportional representation of target sequences due to template re-sampling or unequal template amplification and cloning (Palmer, S. et al., 2005, J Clin Microbiol 43:406-413; Salazar-Gonzalez, J. F. et al., 2008, J Virol 82:3952-3970; Simmonds, P. et al., 1990, J Virol 64:5840-5850; Liu, S. L. et al., 1996, Science 273:415-416).

Based largely on these approaches, previous studies generally described the HCV virus quasispecies in acute and early infection either as relatively “homogeneous,” reflecting transmission of one or few viruses, or relatively “heterogeneous,” reflecting a higher multiplicity of infection. Interestingly, there were parallels between the sequence analysis of HIV-1 and HCV in acute and early infection (Derdeyn, C. A. et al., 2004, Science 303:2019-2022; Frost, S. D. et al., 2005, Proc Natl Acad Sci USA 102:18514-18519; Grobler, J. et al., 2004, J Infect Dis 190:1355-1359; Learn, G. H. et al., 2002, J Virol 76:11953-11959; Long, E. M. et al., 2000, Nat Med 6:71-75; Poss, M. et al., 1995, J Virol 69:8118-8122; Ritola, K. et al., 2004, J Virol 78:11208-11218; Sagar, M. et al., 2004, J Virol 78:7279-7283; Wolfs, T. F. et al., 1992, Virology 189:103-110; Wolinsky, S. M. et al., 1992, Science 255:1134-1137; Zhu, T. et al., 1993, Science 261:1179-1181), but it was not until the development of single genome amplification (SGA)-direct amplicon sequencing that the true nature of the bottleneck to HIV-1 transmission was elucidated (Salazar-Gonzalez, J. F. et al., 2008, J Virol 82:3952-3970; Abrahams, M. R. et al., 2009, J Virol 83:3556-3567; Bar, K. J. et al., 2010, J Virol 84:6241-6247; Haaland, R. E. et al., 2009, PLoS Pathog 5:e1000274; Keele, B. F. et al., 2008, Proc Natl Acad Sci USA 105:7552-7557; Keele, B. F. et al., 2009, J Exp Me, 206:1117-1134; Li, H. et al., 2010, PLoS Pathog 6:e1000890; Salazar-Gonzalez, J. F. et al., 2009, J Exp Med 206:1273-1289). Additionally, an independent obstacle for identifying the transmitted HCV genome is the greater variance in the genome as compared to HIV-1.

Prior studies characterized HCV sequences in acute and early infection using methods that did not involve SGA and direct sequencing, and as a result, they did not allow for precise identification or enumeration of transmitted or early founder viruses, their pathways of diversification as genetic units, or their corresponding phenotypes (Strickland, G. T. et al., 2008, Lancet Infect Dis 8:379-386; Bowen, D. G. et al., 2005, Nature 436:946-952; Cox, A. L. et al., 2005, J Exp Med 201:1741-1752; Kuntzen, T. et al., 2007, J Virol 81:11658-11668; Liu, C. H. et al., 2006, Clin Infect Dis 42:1254-1259; Liu, L. et al., 2010, J Virol 84:5067-5077). The extent to which differences in experimental approach can impact the interpretation of virus complexity was recently highlighted in a study of acute and early HIV-1 subtype C infection (Salazar-Gonzalez, J. F. et al., 2008, J Virol 82:3952-3970) and was seen again in a study in 10 subjects with acute HIV-1 infection who had been analyzed previously by the heteroduplex tracking assay (HTA) method (Ritola, K. et al., 2004, J Virol 78:11208-11218), and for HCV, it was illustrated by 454 deep sequencing (Wang, G. P. et al., 2010, J Virol 84:6218-6228).

Thus, there is a need in the art to develop methods for accurately identifying HCV genome sequences in order to gain a more comprehensive molecular understanding of HCV transmission and for development of effective HCV vaccines and treatments. The present invention provides a novel method based on SGA, direct amplicon sequencing without an interim cloning step, and phylogenetic analysis of sequences with the context of a mathematical model of random virus evolution.

### SUMMARY OF THE INVENTION

The invention provides methods for identifying HCV genomes of transmitted HCV virus (i.e., HCV virus that mediates infection). In the practice of the methods of the invention, improved nucleotide sequence analysis of circulating HCV genomes is utilized for accurate identification of HCV nucleotide sequence. Subsequent phylogenetic analysis by a novel mathematical model disclosed herein provides a means for identifying actual HCV genome nucleotide sequence of transmitted HCV virus. One application of this invention is thus to identify transmitted HCV sequences that mediate viral infection for subsequent development of more effective vaccines and treatments.

More specifically, the invention provides a methods for identifying transmitted HCV genomes, the methods comprising collecting circulating HCV from infected patients, isolating and preparing viral RNA for sequencing, sequencing HCV according to the improved methods provided herein, and performing phylogenetic analysis of said circulating sequences according to mathematical models provided herein to identify actual nucleotide sequence of transmitted HCV virus. In certain embodiments, sequencing of HCV genomes is performed by single genome amplification followed by population sequencing of the DNA amplicons without an interim molecular cloning step. In other embodiments, phylogenetic analysis is performed by a novel mathematical model and/or by alternative phylogenetic methods as described in detail in the Examples below. Ultimately, the methods provide transmitted HCV genomes and nucleotide sequence that are involved in mediating viral infection. In certain embodiments, these sequences include but are not limited to structural proteins and more specifically env and core gene sequences.

The method of the invention further provides for the identification of HCV nucleotide sequence variance over time. Hence, the invention permits evolutionary assessment of transmitted HCV. Such methods are beneficial for the development of vaccines specific to variant polynucleotide and peptide regions of HCV.

The invention provides HCV genomes of transmitted virus (i.e., the nucleotide sequence at the time of viral transmission). In certain embodiments, the polynucleotides of the invention comprise genes encoding HCV structural proteins, including but not limited to env and core genes. Nucleotide sequence of transmitted HCV genomes is set forth in Table I (Appendix A). In additional embodiments, identified transmitted HCV genomes correspond to HCV transmitted in humans, chimpanzees, and other animal models.

In certain embodiments, the invention provides 5′ half-genome HCV sequence and/or full-length genome HCV sequence. More particularly, the invention provides nucleotide sequence comprising one or a plurality of the polynucleotides as set forth in Table I and the Sequence Listing submitted herewith. below. In certain embodiments, the polynucleotides of the invention comprise HCV genes encoding structural proteins, including but not limited to env and core genes. In certain embodiments, the HCV genomes comprise the six global genotypes, subtypes, or drug resistant variants. In additional embodiments, the nucleotide sequences include those identified from circulating HCV virus in addition to nucleotide sequences identified from transmitted HCV virus.

In other aspects, the invention provides methods for producing and administering vaccines comprising HCV nucleotide sequence identified by methods provided herein and/or polypeptides encoded by HCV nucleotide sequence. The advantages of administering the disclosed HCV sequences and/or polypeptides encoded by such HCV sequences is such vaccines would illicit an immune response that is specific to transmitted HCV genomes rather than circulating HCV genomes, thereby improving vaccine effectiveness.

Advantages of this invention include, inter alia, that it permits the identification of HCV genomes of transmitted virus. Prior to this invention actual nucleotide sequence of transmitted HCV genomes was not known due to insufficient methods for error-free sequencing and genetic analysis. The studies disclosed herein illustrate that SGA-direct sequencing unambiguously identifies transmitted viral sequences, which provided a means to characterize core, envelope and full-length HCV genomes and proteins most relevant to virus transmission and in turn, drug and vaccine development. The studies provided in the Examples below provide an understanding of virus natural history and virus-specific cellular and humoral immune responses in naïve and vaccinated individuals.

Specific embodiments of this invention will become evident from the following more detailed description of certain preferred embodiments and the claims.

### BRIEF DESCRIPTION OF THE DRAWINGS

These and other objects and features of this invention will be better understood from the following detailed description taken in conjunction with the drawings, wherein:

FIG. 1 is a schematic representing HCV genome organization. The location of 4.9 kb 5′ half-genome amplicons generated and sequenced herein are at shown bottom left.

FIG. 2 is a schematic representing global HCV genome diversity. The scale bar represents 0.05, or 5%, nucleotide differences between sequences.

FIG. 3 illustrates 5′ half-genome sequence diversity in an acutely-infected subject (10025) productively infected by a single virus. Neighbor-joining phylogenetic tree and a Highlighter plot are shown in the left and right parts of the figure, respectively. The scale bar represents 0.0002, or 1 nucleotide difference between sequences.

FIG. 4 illustrates 5′ half-genome sequence diversity in an acutely-infected subject (10024) productively infected by two viruses. Neighbor-joining phylogenetic tree and a Highlighter plot are shown in the left and right parts of the figure, respectively. The scale bar represents 0.002, or 10 nucleotide differences between sequences.

FIG. 5 illustrates 5′ half-genome sequence diversity in an acutely-infected subject (10021) productively infected by a single virus. Neighbor-joining phylogenetic tree and a Highlighter plot are shown in the left and right parts of the figure, respectively. The scale bar represents 0.0002, or 1 nucleotide difference between sequences. Sequences were derived from plasma sampled over time: Sep. 13, 1998 (gray) and Oct. 4, 1998 (black).

FIG. 6 illustrates 5′ half-genome sequence diversity in an acutely-infected subject (10012) productively infected by three viruses. Neighbor-joining phylogenetic tree and a Highlighter plot are shown in the left and right parts of the figure, respectively. The scale bar represents 0.001, or 5 nucleotide differences between sequences. Sequences were derived from plasma sampled over time: Mar. 17, 1998 (light gray) and Mar. 24, 1998 (dark gray) and 4/5/98 (black).

FIG. 7 illustrates 5′ half-genome sequence diversity in an acutely-infected subject (10029) productively infected by at least eight viruses. Neighbor-joining phylogenetic tree and a Highlighter plot are shown in the left and right parts of the figure, respectively. The scale bar represents 0.002, or 10 nucleotide differences between sequences. Sequences were derived from plasma sampled over time: May 30, 1998 (light gray) and Jun. 14, 1998 (dark gray) and Jun. 28, 1998 (black).

FIG. 8 illustrates 5′ half-genome sequence diversity in an acutely-infected subject (10020) productively infected by more than two viruses. Neighbor-joining phylogenetic tree and a Highlighter plot are shown in the left and right parts of the figure, respectively. The scale bar represents 0.0002, or 1 nucleotide difference between sequences. Sequences were derived from plasma sampled on Sep. 27, 1998 (gray) and Oct. 22, 1998 (black).

FIG. 9 illustrates 5′ half-genome sequence diversity in a chronically-infected subject (JOTO6642). Neighbor-joining phylogenetic tree and a Highlighter plot are shown in the left and right parts of the figure, respectively. The amplified and sequenced region of the genome is represented at the bottom of the figure. The scale bar represents 0.002, or 10 nucleotides difference between sequences.

FIG. 10 illustrates 5′ half-genome sequence diversity in a chronically-infected subject (WHRO3382). Neighbor-joining phylogenetic tree and a Highlighter plot are shown in the left and right parts of the figure. The scale bar represents 0.001, or 5 nucleotides difference between sequences.

### DETAILED DESCRIPTION OF THE PREFERRED EMBODIMENTS

The invention provides methods for identifying HCV genomes that mediate viral infection and transmission. More particularly, the methods provide a means for identifying specific nucleotide sequences of transmitted HCV virus, which prior to the present invention were unavailable due to the following: (i) a significant time period from weeks to months between the moment of transmission and the first appearance of HCV in the blood (Bowen. D. G. and Walker, C. M., 2005, Nature 436: 946-952; Moradpour, D. et al., 2007, Nature Rev Micro 5:453-463); (ii) HCV is genetically highly variable in its nucleotide sequence due to its error-prone RNA-dependent RNA polymerase, and as a result, it exists in individuals as a complex mixture of sequences commonly referred to as a ‘quasispecies’ (Moradpour, D. et al., 2007, Nature Rev Micro 5:453-463); (iii) conventional experimental approaches to sequencing the HCV genome from clinical samples introduced addition variation into the sequences as a consequence of Taq polymerase induced recombination and nucleotide misincorporation errors (Salazar-Gonzalez, J. F. et al., 2008, J Virol 82:3952-3970); and (iv) identifying from these myriad of sequences which sequences corresponded to actual transmitted viruses, or even viruses that were replication-competent and responsible for ongoing virus replication and persistence in the infected subject was not achievable. The identification of transmitted viral sequence provides specific nucleotide and protein regions responsible for mediating viral infection. Utilizing these specific regions in the preparation of vaccines and therapeutic provides for more effective HCV treatments.

In one embodiment, the methods comprise: (a) collecting a patient sample; (b) isolating viral RNA from said sample; (c) sequencing said viral RNA, wherein viral RNA sequencing includes HCV genomes of circulating virus; (d) performing sequence alignment of selected HCV genome regions; (e) analyzing phylogenetically selected sequence alignments; and (f) identifying HCV genomes of transmitted virus. In a further embodiment, the method comprises the additional step (g) detecting the variation in transmitted viral sequence over time, wherein variations in said viral sequences are identified.

The invention is not limited to the identification of transmitted viral genomes, but also includes the identification of circulating viral genomes. The sequence analysis of circulating HCV genomes provides raw data for subsequent phylogenetic analysis. For example, see SEQ ID NOS: 23-757. While the identification of transmitted HCV genomes is a preferred embodiment, the invention is not limited to that example.

As used herein, a “patient” or “subject” to be utilized by the disclosed methods can mean a human, chimpanzee or non-human primate. In certain embodiments the patient is a non-human mammal.

The term “patient sample” as used herein includes but is not limited to a blood, serum, plasma, or urine sample obtained from a patient. In particular embodiments, the patient sample is plasma. In a preferred embodiment, the patient is infected with HCV.

The phrase “performing phylogenetic analysis” as used herein represents the analysis of clinical viral isolates, regardless of the particular methodology employed. Comparative analysis of the genetic relatedness of any a collection of circulating viral isolates is used to select for nucleotide sequence of transmitted HCV genomes. This methodology is used to select for the actual HCV genomes present at the time of patient infection/viral transmission. This can be accomplished by any number of methods, including but not limited to: i) a novel mathematical model of random virus evolution disclosed herein; ii) star phylogeny; iii) Baysian analysis; or any other method of phylogenetic analysis known to one of skill in the art.

The phrase “performing sequence alignment” as used herein is meant to include aligning genetic sequences by any of a number of different procedures that produce a sufficient match between the corresponding residue in the sequences. Typically, Smith-Waterman or Needleman-Wunsch algorithms are used. However, other procedures such as BLAST, FASTA, PSI-BLAST can be used. In certain embodiments manual alignment (i.e., performed by a technician reading the actual sequence and selected the alignment) is performed.

The phrase “isolating viral RNA” as used herein includes those methods well known in the art for extraction of viral RNA from a sample, cDNA synthesis from viral RNA template, optionally cloning of cDNA fragments, and/or amplification of polynucleotide sequence. Such methods are described in more detail, but such experimental procedures are provided in Sambrook and Russell, 2001, Molecular Cloning: A Laboratory Manual, Third Ed., Cold Spring Harbor Laboratory Press, Woodbury, N.Y.

The practice of this invention can involve procedures well known in the art, including for example nucleotide sequence amplification, such as polymerase chain reaction (PCR) and modifications thereof (including for example reverse transcription (RT)-PCR, and stem-loop PCR), as well as reverse transcription and in vitro transcription. Generally these methods utilize one or a pair of oligonucleotide primers having sequence complimentary to sequences 5′ and 3′ to the sequence of interest, and in the use of these primers they are hybridized to a nucleotide sequence and extended during the practice of PCR amplification using DNA polymerase (preferably using a thermal-stable polymerase such as Taq polymerase). RT-PCR may be performed on miRNA or mRNA with a specific 5′ primer or random primers and appropriate reverse transcription enzymes such as avian (AMV-RT) or murine (MMLV-RT) reverse transcriptase enzymes.

In a preferred embodiment, methods of the invention utilize single genome amplification (SGA) for improved sequence accuracy instead of the traditional cloning and amplification methods described immediately above. Details of this method are provided in Example 3, B. Keele et al., 2008, Proc Natl Acad Sci USA 105:7552-57; and Salazar-Gonzalez et al., 2009, J Exp Med 206: 1273-1289. Advantages of SGA are described herein, and include reduced error rate. SGA followed by direct sequencing of uncloned amplicon DNA mitigates Taq polymerase nucleotide misincorporation, thereby resulting in nucleotide sequence that more closely relates to the original viral RNA template.

The phrase “over time” as used herein represents a period of time between the collection of patient samples. For example, a patient sample is collected at time point A and then subsequently as a later date at time point B. The period between samplings is over time. In certain embodiments, samples are taken from the same patient. In alternative embodiments, samples may be taken from different patients. Performing the method of the invention at different time points followed by the differential analysis of HCV genomes at the different time points provides a means for assessing the evolution of HCV genomes both inter- and intra-patient. The identification of highly variable genomic regions is useful for the generation of effective therapeutics and vaccines to HCV.

The term “transmitted” as used herein refers to viral genotype and phenotype at the time of viral infection. The term “transmitted virus” as used here is in reference to actual HCV virus that mediates patient infection. The phrase “transmitted viral sequence” as used herein is meant to include nucleotide or amino acid sequence of HCV genomes of transmitted virus. The term “circulating” refers to HCV virus collected from patient post-infection. In general, patient samples comprise circulating virus because HCV infection has occurred at a prior time point. The half-life of plasma virus is less than 1 day, thus the collection of transmitted virus from a patient sample would be a rare event.

The term “selected” as used in the phrases “selected sequence alignments” or “selected HCV genome regions” is meant to include the identification and utilization of particular subsets of nucleotide sequence and/or genome regions for subsequent analysis. In certain embodiments, full-length HCV genomes are identified, however discrete portions of the genome are utilized or “selected” for further analysis.

In one embodiment, the full-length HCV genome of transmitted virus is identified by the methods of the invention. In another embodiment, half-genomes of transmitted and circulating HCV are identified. In certain embodiments, the regions corresponding to HCV structural genes, including env and core genes are identified. See Table I and Sequence Listing (SEQ ID NOS: 1-757).

The invention also includes polynucleotide sequences identified by the methods disclosed herein. Transmitted HCV sequences are provided in Table I. A further embodiment of the invention includes polypeptides encoded by the polynucleotides of Table I. The polynucleotide and polypeptides of transmitted HCV provide the basis for effective vaccine development.

The present invention also provides vaccine compositions and methods for delivery of HCV polynucleotide or polypeptide sequences to a vertebrate with optimal expression and safety conferred. These vaccine compositions may be prepared and administered in such a manner that the encoded gene products are optimally expressed in the vertebrate of interest. As a result, these compositions and methods are useful in stimulating an immune response against HCV infection. Also included in the invention are expression systems and delivery systems.

In a specific embodiment, the invention provides polynucleotide (e.g., DNA) vaccines (Wei, et al., 2010, Science 329:1060-64) as well as combinatorial vaccines which combine both a polynucleotide vaccine and polypeptide (e.g., either a recombinant protein, a purified subunit protein, a viral vector expressing an isolated HCV polypeptide, or in the form of an inactivated or attenuated HCV vaccine) vaccine in a single formulation, or polypeptide vaccines. The formulation comprises a polynucleotide, or an HCV polypeptide-encoding polynucleotide vaccine, and optionally, an effective amount of a desired isolated HCV polypeptide or fragment, variant, or derivative thereof. The polypeptide may exist in any form, for example, a recombinant protein, a purified subunit protein, a viral vector expressing an isolated HCV polypeptide, or in the form of an inactivated or attenuated HCV vaccine. The HCV polypeptide or fragment, variant, or derivative thereof encoded by the polynucleotide vaccine may be identical to the isolated HCV polypeptide or fragment, variant, or derivative thereof. Alternatively, the HCV polypeptide or fragment, variant, or derivative thereof encoded by the polynucleotide may be different from the isolated HCV polypeptide or fragment, variant, or derivative thereof.

Transmitted HCV genome components can be administered as DNA plasmids for DNA vaccination (Id.), as recombinant proteins, genes expressed in vectors including replication deficient recombinant adenovirus (Barefoot, B. et al., 2008, Vaccine 26: 6108-18), recombinant mycobacteria (Id.; Yu et al., 2007, Clinical Vaccine Immunol 14; 886-893; Yu, et al., 2006, Clinical Vaccine Immunol, 13: 1204-11, Larsen et al., 2009, Vaccine 27: 4709-17), recombinant vesicular stomatititis virus, recombinant vaccinia virus or variants thereof such as modified vaccinia Ankara (Sun et al., 2010, Virology 406: 48-55; Estaban, M., 2009, Human Vaccines 5: 867-71) or NYVAC (Estaban, M., 2009, Human Vaccines 5: 867-71). Advantageously, hepatitis C envelope genes or proteins can be used to induce neutralizing antibody responses either as proteins used alone, as DNAs or vectors used alone or in a DNA prime vector boost regimen, or as a vector prime protein boost regimen. Recombinant proteins may be used as primes and/or boosts with certain adjuvants. Adjuvants include monophosphoryl lipid A, MF59, Alum as well as various oil in water or water in oil emulsions, as examples (Mbow, et al., 2010, Current Opinion in Immunology 22: 411-416). Finally, hepatitis C envelope can be expressed as virus like particles and administered either alone or in adjuvant formulations (Qiao et al., 2003, Hepatology 37: 52-9). Administration routes include for example, intramuscularly, subcutaneously, and/or mucosally, most advantageously intranasally.

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

The term “polynucleotide” is intended to encompass a singular nucleic acid or nucleic acid fragment as well as plural nucleic acids or nucleic acid fragments, and refers to an isolated molecule or construct, e.g., a virus genome (e.g., vRNA), messenger RNA (mRNA), plasmid DNA (pDNA), or derivatives of pDNA (e.g., minicircles as described in (Darquet, A-M et al., 1997, Gene Therapy, 4:1341-1349) comprising a polynucleotide. A polynucleotide may comprise a conventional phosphodiester bond or a non-conventional bond (e.g., an amide bond, such as found in peptide nucleic acids (PNA)).

The terms “nucleic acid” or “nucleic acid fragment” refer to any one or more nucleic acid segments, e.g., DNA or RNA fragments, present in a polynucleotide or construct. A nucleic acid or fragment thereof may be provided in linear (e.g., mRNA) or circular (e.g., plasmid) form as well as double-stranded or single-stranded forms. By “isolated” nucleic acid or polynucleotide is intended a nucleic acid molecule, DNA or RNA, which has been removed from its native environment. For example, a recombinant polynucleotide contained in a vector is considered isolated or “cloned” for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of the polynucleotides of the present invention. Isolated polynucleotides or nucleic acids according to the present invention further include such molecules produced synthetically.

The terms “fragment,” “variant,” and “derivative” when referring to HCV polypeptides of the present invention include any polypeptides which retain at least some of the immunogenicity or antigenicity of the corresponding native polypeptide. Fragments of HCV polypeptides of the present invention include proteolytic fragments, deletion fragments and in particular, fragments of HCV polypeptides which exhibit increased secretion from the cell or higher immunogenicity or reduced pathogenicity when delivered to an animal. Polypeptide fragments further include any portion of the polypeptide which comprises an antigenic or immunogenic epitope of the native polypeptide, including linear as well as three-dimensional epitopes. Variants of HCV polypeptides of the present invention include fragments, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome or genome of an organism or virus. Genes II, Lewin, B., ed., John Wiley & Sons, New York (1985), which is incorporated herein by reference. For example, as used herein, variations in a given gene product is a “variant”. Naturally or non-naturally occurring variations such as amino acid deletions, insertions or substitutions may occur. Non-naturally occurring variants may be produced using art-known mutagenesis techniques. Variant polypeptides may comprise conservative or non-conservative amino acid substitutions, deletions or additions. Derivatives of HCV polypeptides of the present invention, are polypeptides which have been altered so as to exhibit additional features not found on the native polypeptide. Examples include fusion proteins. An analog is another form of an HCV polypeptide of the present invention. An example is a proprotein which can be activated by cleavage of the proprotein to produce an active mature polypeptide.

In certain embodiments, the polynucleotide, nucleic acid, or nucleic acid fragment is DNA. In the case of DNA, a polynucleotide comprising a nucleic acid which encodes a polypeptide normally also comprises a promoter and/or other transcription or translation control elements operably associated with the polypeptide-encoding nucleic acid fragment. An operable association is when a nucleic acid fragment encoding a gene product, e.g., a polypeptide, is associated with one or more regulatory sequences in such a way as to place expression of the gene product under the influence or control of the regulatory sequence(s). Two DNA fragments (such as a polypeptide-encoding nucleic acid fragment and a promoter associated with the 5′ end of the nucleic acid fragment) are “operably associated” if induction of promoter function results in the transcription of mRNA encoding the desired gene product and if the nature of the linkage between the two DNA fragments does not (1) result in the introduction of a frame-shift mutation, (2) interfere with the ability of the expression regulatory sequences to direct the expression of the gene product, or (3) interfere with the ability of the DNA template to be transcribed. Thus, a promoter region would be operably associated with a nucleic acid fragment encoding a polypeptide if the promoter was capable of effecting transcription of that nucleic acid fragment. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, elements from picornaviruses (particularly an internal ribosome entry site, or IRES, also referred to as a CITE sequence).

A DNA polynucleotide of the present invention may be a circular or linearized plasmid or vector, or other linear DNA which may also be non-infectious and nonintegrating (i.e., does not integrate into the genome of vertebrate cells). A linearized plasmid is a plasmid that was previously circular but has been linearized, for example, by digestion with a restriction endonuclease. Linear DNA may be advantageous in certain situations as discussed, e.g., in Cherng, J. Y., et al., 1999, J. Control. Release 60:343-53, and Chen, Z. Y., et al., 2001, Mol Ther 3:403-10. As used herein, the terms plasmid and vector can be used interchangeably. In other embodiments, a polynucleotide of the present invention is RNA, for example, in the form of messenger RNA (mRNA). Methods for introducing RNA sequences into vertebrate cells are described in U.S. Pat. No. 5,580,859.

Polynucleotides, nucleic acids, and nucleic acid fragments of the present invention may be associated with additional nucleic acids which encode secretory or signal peptides, which direct the secretion of a polypeptide encoded by a nucleic acid fragment or polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted, or “mature” form of the polypeptide. In certain embodiments, the native leader sequence is used, or a functional derivative of that sequence that retains the ability to direct the secretion of the polypeptide that is operably associated with it. Alternatively, a heterologous mammalian leader sequence, or a functional derivative thereof, may be used. For example, the wild-type leader sequence may be substituted with the leader sequence of human tissue plasminogen activator (TPA) or mouse beta-glucuronidase.

In accordance with one aspect of the present invention, there is provided a polynucleotide construct, for example, a plasmid, comprising a nucleic acid fragment. As used herein, the term “plasmid” refers to a construct made up of genetic material (i.e., nucleic acids). Typically a plasmid contains an origin of replication which is functional in bacterial host cells, e.g., Escherichia coli, and selectable markers for detecting bacterial host cells comprising the plasmid. Plasmids of the present invention may include genetic elements as described herein arranged such that an inserted coding sequence can be transcribed and translated in eukaryotic cells. Also, the plasmid may include a sequence from a viral nucleic acid. However, such viral sequences normally are not sufficient to direct or allow the incorporation of the plasmid into a viral particle, and the plasmid is therefore a non-viral vector. In certain embodiments described herein, a plasmid is a closed circular DNA molecule.

The term “expression” refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

### Polypeptides and Immunogenic Epitopes

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” can be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

Also included as polypeptides of the present invention are fragments, derivatives, analogs, or variants of the foregoing polypeptides, and any combination thereof. Polypeptides, and fragments, derivatives, analogs, or variants thereof of the present invention can be antigenic and immunogenic polypeptides related to HCV polypeptides, which are used to prevent or treat, i.e., cure, ameliorate, lessen the severity of, or prevent or reduce contagion of infectious disease caused by the HCV.

As used herein, an “antigenic polypeptide” or an “immunogenic polypeptide” is a polypeptide which, when introduced into a vertebrate, reacts with the vertebrate's immune system molecules, i.e., is antigenic, and/or induces an immune response in the vertebrate, i.e., is immunogenic. It is quite likely that an immunogenic polypeptide will also be antigenic, but an antigenic polypeptide, because of its size or conformation, may not necessarily be immunogenic. Isolated antigenic and immunogenic polypeptides of the present invention in addition to those encoded by polynucleotides of the invention, may be provided as a recombinant protein, a purified subunit, a viral vector expressing the protein, or may be provided in the form of an inactivated HCV vaccine, e.g., a live-attenuated virus vaccine, a heat-killed virus vaccine, etc.

By an “isolated” HCV polypeptide or a fragment, variant, or derivative thereof is intended an HCV polypeptide or protein that is not in its natural form. No particular level of purification is required. For example, an isolated HCV polypeptide can be removed from its native or natural environment. Recombinantly produced HCV polypeptides and proteins expressed in host cells are considered isolated for purposed of the invention, as are native or recombinant HCV polypeptides which have been separated, fractionated, or partially or substantially purified by any suitable technique, including the separation of HCV virions from culture cells in which they have been propagated. In addition, an isolated HCV polypeptide or protein can be provided as a live or inactivated viral vector expressing an isolated HCV polypeptide and can include those found in inactivated HCV vaccine compositions. Thus, isolated HCV polypeptides and proteins can be provided as, for example, recombinant HCV polypeptides, a purified subunit of HCV, a viral vector expressing an isolated HCV polypeptide, or in the form of an inactivated or attenuated HCV vaccine.

The term “epitopes,” as used herein, refers to portions of a polypeptide having antigenic or immunogenic activity in a vertebrate, for example a human. An “immunogenic epitope,” as used herein, is defined as a portion of a protein that elicits an immune response in an animal, as determined by any method known in the art. The term “antigenic epitope,” as used herein, is defined as a portion of a protein to which an antibody or T-cell receptor can immunospecifically bind as determined by any method well known in the art. Immunospecific binding excludes non-specific binding but does not exclude cross-reactivity with other antigens. Where all immunogenic epitopes are antigenic, antigenic epitopes need not be immunogenic.

As to the selection of peptides or polypeptides bearing an antigenic epitope (e.g., that contain a region of a protein molecule to which an antibody or T cell receptor can bind), it is well known in that art that relatively short synthetic peptides that mimic part of a protein sequence are routinely capable of eliciting an antiserum that reacts with the partially mimicked protein. See, e.g., Sutcliffe, J. G., et al., 1983, Science 219:660-666.

### Vaccine Compositions and Administration

The identified polynucleotides or polypeptides encoded by the polynucleotides of the invention may be in any form, and polypeptides are generated using techniques well known in the art. Examples include isolated HCV proteins produced recombinantly or proteins delivered in the form of an inactivated HCV vaccine, such as conventional vaccines.

When utilized, an isolated HCV polynucleotide or polypeptide or fragment, variant or derivative thereof is administered in an immunologically effective amount. The effective amount of conventional vaccines is determinable by one of ordinary skill in the art based upon several factors, including the antigen being expressed, the age and weight of the subject, and the precise condition requiring treatment and its severity, and route of administration.

In the instant invention, the combination of conventional antigen vaccine compositions with optimized nucleic acid or polypeptide compositions provides for therapeutically beneficial effects at dose sparing concentrations. For example, immunological responses sufficient for a therapeutically beneficial effect in patients predetermined for an approved commercial product, such as for the conventional product described above, can be attained by using less of the approved commercial product when supplemented or enhanced with the appropriate amount of nucleic acid or polypeptide.

A desirable level of an immunological response afforded by a DNA based pharmaceutical alone may be attained with less DNA by including an aliquot of a conventional vaccine. Further, using a combination of conventional and DNA based pharmaceuticals may allow both materials to be used in lesser amounts while still affording the desired level of immune response arising from administration of either component alone in higher amounts (e.g. one may use less of either immunological product when they are used in combination). This may be manifest not only by using lower amounts of materials being delivered at any time, but also to reducing the number of administrations points in a vaccination regime (e.g. 2 versus 3 or 4 injections), and/or to reducing the kinetics of the immunological response (e.g. desired response levels are attained in 3 weeks in stead of 6 after immunization).

Determining the precise amounts of DNA based pharmaceutical and conventional antigen is based on a number of factors as described above, and is readily determined by one of ordinary skill in the art.

The ability of an adjuvant to increase the immune response to an antigen is typically manifested by a significant increase in immune-mediated protection. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion.

Nucleic acid molecules and/or polynucleotides of the present invention, e.g., plasmid DNA, mRNA, linear DNA or oligonucleotides, may be solubilized in any of various buffers. Suitable buffers include, for example, phosphate buffered saline (PBS), normal saline, Tris buffer, and sodium phosphate (e.g., 150 mM sodium phosphate). Insoluble polynucleotides may be solubilized in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art. For aqueous compositions used in vivo, sterile pyrogen-free water can be used. Such formulations will contain an effective amount of a polynucleotide together with a suitable amount of an aqueous solution in order to prepare pharmaceutically acceptable compositions suitable for administration to a human.

Compositions of the present invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995). Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

This invention is more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined below to provide additional guidance to the practitioner regarding the description of the invention.

### EXAMPLES

The Examples which follow are illustrative of specific embodiments of the invention, and various uses thereof. They are set forth for explanatory purposes only, and are not to be taken as limiting the invention.

### Example 1

### Identification of Transmitted HCV Genomes

In addition, the invention is not intended to be limited to the disclosed embodiments of the invention. It should be understood that the foregoing disclosure emphasizes certain specific embodiments of the invention and that all modifications or alternatives equivalent thereto are within the spirit and scope of the invention as set forth in the appended claims.

In an effort to identify the structural genes of HCV viruses at the time of actual viral transmission and patient infection, a novel method for unambiguously identifying the transmitted 5′ half-genome and core and env genes of HCV viruses was developed. The identification of transmitted HCV genomes not only provides an important structural and genetic information at the time of viral transmission, it also permits a broader understating of HCV infection and provides a means for tracking evolution in the critical period between transmission, peak viremia and antibody seroconversion and thereafter (e.g., some infected patients develop persistent infection/disease whereas others experience a spontaneous or treatment-related complete clinical remission or cure).

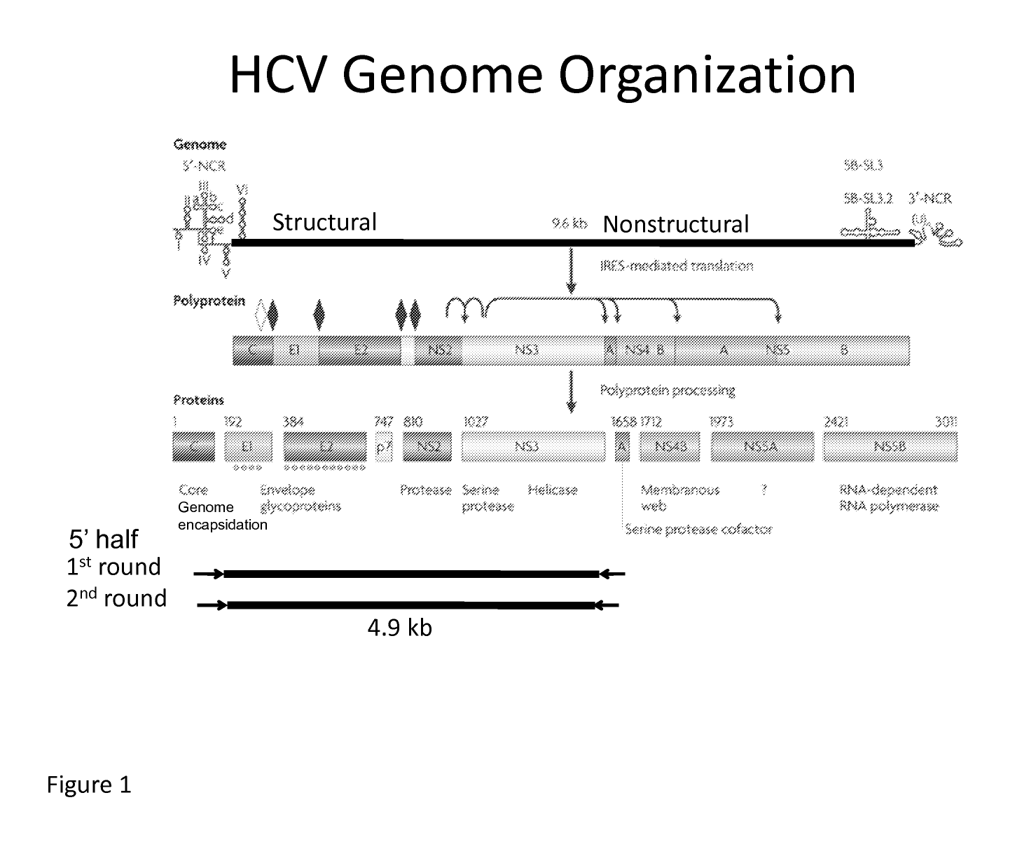

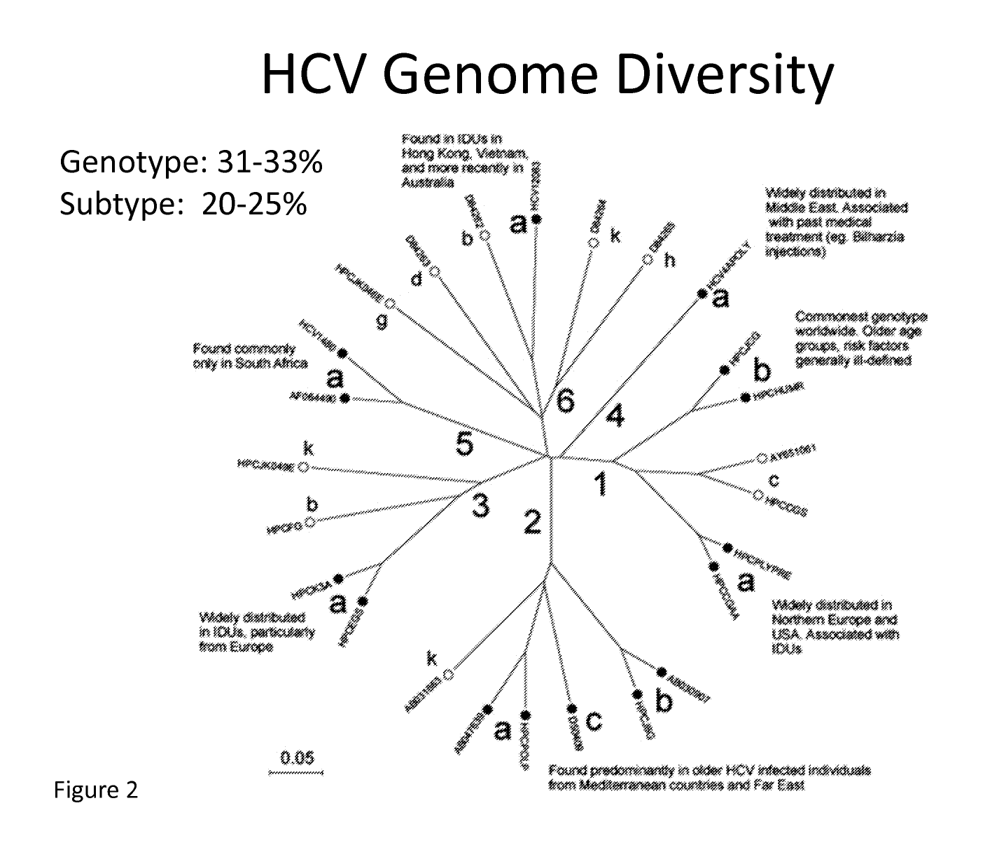

Until the present method, the identification of actual transmitted HCV genomes has not been possible. The complete HCV genome of ˜9.6 kb in length (Moradpour, D., et al., 2007, Nature Rev Micro 5:453-463) is represented in FIG. 1 and the six globally-circulating genetic lineages (Simmonds, P., 2004, J Gen Virol 85:3173-3188) are represented in FIG. 2. The reasons that actual transmitted HCV genomes could not be identified were several-fold: First, there is a period lasting weeks and sometimes months between the moment of transmission and the first appearance of HCV in the blood (Bowen. D. G. and Walker, C. M., 2005, Nature 436: 946-952; Moradpour, D., et al., 2007, Nature Rev Micro 5:453-463). Second, HCV is genetically highly variable in its nucleotide sequence due to its error-prone RNA-dependent RNA polymerase; as a result, it exists in individuals as a complex mixture of sequences commonly referred to as a ‘quasispecies’ (Moradpour, D. et al., 2007, Nature Rev Micro 5:453-463). Third, conventional experimental approaches to sequencing the HCV genome from clinical samples introduced addition variation into the sequences as a consequence of Taq polymerase induced recombination and nucleotide misincorporation errors (Salazar-Gonzalez, J. F. et al., 2008, J Virol 82:3952-3970). Four, even if relatively accurate sequencing of HCV genomes were generated from clinical samples, there was no way to deduce from these myriad of sequences which one (or which ones) corresponded to actual transmitted viruses, or even viruses that were replication-competent and responsible for ongoing virus replication and persistence in the infected subject. Because transmitted viral molecular clones were not available, and because virus could not be isolated in vitro from clinical samples, biological analysis was challenging.

Some progress was made in 2003 when a molecular clone of an HCV genome was obtained from a patient with fulminant hepatitis C infection Kato, T. et al., 2003, Gastroenterology 125:1808-1817). This viral clone did not correspond to an actual transmitted virus genome, but it did replicate in human liver cell lines in vitro. Improvements in the replication of this clone have since been made by making genetic chimeras with other subgenomic HCV fragments (Moradpour, D., et al., 2007, Nature Rev Micro 5:453-463), but none of these clones of HCV correspond to transmitted viral genomes and none of them reproduce the genetic content or biological properties of actual transmitted viruses that are responsible for transmission and clinical infection in humans. Still another limitation of these clones is that the do not represent the six major genetic lineages and many more subtypes of HCV that circulate globally. For all these reasons, there is an urgent need for a method to identify the exact nucleotide sequence of transmitted HCV genomes. The present invention provides a method, which is based on SGA, direct amplicon sequencing without an interim cloning step, and a phylogenetic analysis of sequences with the context of a mathematical model of random virus evolution. The methods provided herein implemented a means for identifying transmitted HCV genomes. First, single genome amplification (SGA) of HIV-1 plasma viral RNA (Salazar-Gonzalez, J. F. et al., 2008, J Virol 82:3952-3970; Keele, B. F. et al., 2008, Proc Natl Acad Sci USA 105:7552-7557) followed by direct sequencing of uncloned amplicon DNA precludes Taq-induced nucleotide misincorporation and recombination in finished sequences. Second, because of the extremely short in vivo lifespan of plasma virus (t_{1/2}<1 day), (Neumann, A. U. et al., 1998, Science 282:103-107) analysis of plasma vRNA could provide a uniquely informative view of HCV replication dynamics and evolution, thereby allowing for a phylogenetic identification of the transmitted viral genome(s).

Thus, a method was devised utilizing SGA-based analysis of plasma vRNA obtained from acutely infected individuals in the earliest stages of infection, which was evaluated within the context of a model of random viral evolution. This method allowed the identification of nucleotide sequences of core genes, env genes and 5′ half-genomes of viruses responsible for establishing productive clinical infection weeks earlier (Table I).

### Example 2

### Mathematical Model

A new mathematical model for HCV was constructed based on (Keele, B. F. et al., 2008, Proc Natl Acad Sci USA 105:7552-7557; Lee, H. Y. et al., 2009, J Theor Biol 261:341-360) previously estimated parameters of HIV-1 generation time (2 days) (Markowitz, M. et al., 2003, J Virol 77:5037-5038) reproductive ratio (R₀, 6) (Stafford, M. A. et al., 2000, J Theor Biol, 203:285-301), reverse transcriptase (RT) error rate (2.16×10⁻⁵) (41), and an assumption that the initial virus replicates exponentially infecting R₀ new cells at each generation and diversifying under a model of evolution that assumes no selection. A comparable and novel model of HCV replication and diversification was developed based on derivatives of these parameters. Under both models, viruses would exhibit a Poisson distribution of mutations and a star-like phylogeny (Slatkin, M. et al., 1991, Genetics 129:555-562). These models allowed the assessment of the following two points: (i) the progeny of individual virus(es) that establish productive infection are identified in early stages of HCV infection as distinct genetic lineages with low sequence diversity; and (ii) the consensus sequence of each env lineage sampled prior to the onset of immune selection corresponds to the actual env sequence of transmitted or founder virus (or viruses) responsible for establishing productive clinical infection. Details of the models utilized for diversity analysis are provided in Example 5.

### Example 3

### HCV Patient Selection and Analysis

HCV patients were selected and samples collected as described. All subjects were regular donors of source plasma for whom serial specimens were available for analysis. As part of routine blood banking practice, these individuals were regularly questioned (and deferred) for homosexual encounters, sex for money, or intravenous drug use and they were monitored for acquisition of blood-born infectious agents (including HIV) that might indicate risk factors for HCV acquisition, but it is likely nonetheless that the cause of virus transmission was by one of these routes or by other types of sexual exposures. All subjects were plasma HCV RNA positive and HCV antibody negative.

Plasma samples were obtained from subjects with acute or very recent HCV infection representing subtypes 1a, 1b, 2 or 3. These consisted of weekly or twice-weekly serial collections from source plasma donors who became HCV infected during the course of their plasma donations. Plasma samples from 10 subjects with chronic HCV infection from the U.S. served as controls. All subjects gave informed consent, and plasma collections were performed with institutional review board and other regulatory approvals. Blood specimens were collected in acid citrate dextrose and plasma separated and stored at −20 to −70° C. Plasma samples were tested for HCV RNA and viral specific antigen and antibodies by a battery of commercial tests (Abbott; Chiron; Roche).

Differences in risk behaviors, routes of virus transmission, clinical stage and viral load in the infected partner, and co-morbid clinical conditions can all influence the frequency of HCV transmission (Kleinman, S. H. et al., 2009, Transfusion 49:2454-2489) and likely could affect the numbers of viruses transmitted and subsequent disease natural history, factors relevant to vaccine design and evaluation. In the present study, subjects had limited behavioral information available for analysis, so we could draw no firm conclusions regarding particular risk factors leading to infection.

Sequencing and analysis of 646 5′ half genome sequences from plasma vRNA from 10 subjects acutely infected with HCV genetic lineages 1, 2 and 3 was performed. To ensure proportional representation of plasma vRNA and avoid in vitro generated recombination events and Taq polymerase errors, SGA of plasma vRNA followed by direct sequence analysis of uncloned 5′ half-genome amplicons was performed (Palmer, S. et al., 2005, J Clin Microbiol 43:406-413; Salazar-Gonzalez, J. F. et al., 2008, J Virol 82:3952-3970; Shriner, D. et al., 2004, Genetics 167:1573-1583; Keele, B. F. et al., 2008, Proc Natl Acad Sci USA 105:7552-7557; Keele, B. F. et al., 2009, J Exp Med 206:1117-1134; Salazar-Gonzalez, J. F. et al., 2009, J Exp Med 206:1273-1289). Sequences were excluded if the chromatogram revealed “double peaks,” indicative of amplification from more than one template or early Taq polymerase error. The number of sequences analyzed per subject ranged from 25-68). The maximum within-patient 5′ half-genome sequence diversity ranged from 0.08% to 6.82%, with very low diversity found in 4 subjects (0.08-0.20%) and distinctly higher diversity found in 6 others (range 1.03-6.82%).

Viral RNA isolation and cDNA synthesis was performed as follows. For each sample, approximately 200 ul plasma was extracted using the QIAamp Viral RNA Mini Kit (Qiagen, Valencia, Calif.). RNA was eluted and immediately subjected to cDNA synthesis. Reverse transcription of RNA to single stranded cDNA was performed using SuperScript III reverse transcriptase using methods recommended by the manufacturer (Invitrogen Life Technologies, Carlsbad, Calif.). Briefly, each cDNA reaction included 1×RT buffer, 0.5 mM of each deoxynucleoside triphosphate, 5 mM dithiothreitol, 2 units/μl RNaseOUT (RNase inhibitor), 10 units/μl of SuperScript III reverse transcriptase, and 0.25 μM antisense primer. The antisense primers were designed specifically for different genotype. 1.NS4A-R1 5′-GCACTCTTCCATCTCATCGAACTC-3′(SEQ ID NO: 758) (nt 5451-5474 H77 (accession number NC_{—}004102)) for genotype 1, 2NS4A-R1 5′-TCCATCTCATCAAARGCCTCATA-3′ (SEQ ID NO:759) (nt 5445-5467 H77) for genotype 2 and 3aNS3-R2V2 5′-TTACTTCCAGATCAGCTGACA-3′(SEQ ID NO:760) for genotype 3. The mixture was incubated at 50° C. for 60 minutes followed by an increase in temperature to 55° C. for an additional 60 minutes. The reaction was then heat-inactivated at 70° C. for 15 minutes and then treated with RNaseH at 37° C. for 20 minutes. The newly synthesized cDNA was used immediately or kept frozen at −80° C.

Single genome amplification was performed from prepared viral cDNA. cDNA was serially diluted and distributed among wells of replicate 96-well plates so as to identify a dilution where PCR positive wells constituted less than 30% of the total number of reactions. At this dilution, most wells contain amplicons derived from a single cDNA molecule. This was confirmed in every positive well by direct sequencing of the amplicon and inspection of the sequence for mixed bases (double peaks), which would be evidence of priming from more than one original template or the introduction of PCR error in early cycles. Any sequence with evidence of mixed bases was excluded from further analysis. PCR amplification was carried out in the presence of 1× High Fidelity Platinum PCR buffer, 2 mM MgSO₄, 0.2 mM of each deoxynucleoside triphosphate, 0.2 μM of each primer, and 0.025 units/μl Platinum Taq High Fidelity polymerase in a 20 μl reaction (Invitrogen, Carlsbad, Calif.).

The nested or hemi-nested primers for generating 5′ half genome from different genotypes included: (1) genotype 1: 1^{st} round sense primer 1.core.F1 5′-ATGAGCACGAATCCTAAACCTCAAAGA-3′ (SEQ ID NO:761) (nt 342-368 H77) or 1.5utr.F1 5′-TGGGGGCGACACTCCACCAT-3′ (SEQ ID NO:762) (nt 14-33 H77) and 1^{st} round antisense primer 1.NS4A.R1 5′-GCACTCTTCCATCTCATCGAACTC-3′ (SEQ ID NO:763) (nt 5451-5474 H77), 2^{nd} round sense primer 1.core.F2 5′-TCAAAGAAAAACCAAACGTAACACCAACCG-3′ (SEQ ID NO:764) (nt 362-391 H77) or 1.5utr.F2 5′-CACCATAGATCACTCCCCTGTGAGGAACTA-3′ (SEQ ID NO:765) (nt 28-57 H77) and 2^{nd} round antisense primer 1.NS3A4A.R2 5′-AGGTGCTCGTGACGACCTCCAGG-3′ (SEQ ID NO:766) (nt 5297-5319 H77); (2) genotype 2: 1^{st} round sense primer 2.core.F1 5′-ATGAGCACAAATCCTAAACCTCAAAGA-3′ (SEQ ID NO:767) (nt 342-368 H77) and 1^{st} round antisense primer 2.NS4A.R1 5′-TCCATCTCATCAAARGCCTCATA-3′ (SEQ ID NO:768) (nt 5445-5467 H77), 2^{nd} round sense primer 2.core.F2 5′-AATCCTAAACCTCAAAGAAAAACCAAA-3′ (SEQ ID NO:769) (nt 351-377 H77) and 2^{nd} round antisense primer 2.NS3A4A.R2 5′-GACCTCAAGGTCAGCTTGCAT-3′(SEQ ID NO:770); (3) genotype 3: 1^{st} round sense primer 3a.core.F1 5′-ATGAGCACACTTCCTAAACCTCAAAGA-3′ (SEQ ID NO:771) and 1^{st} round antisense primer 3aNS3-R2V2 5′-TTACTTCCAGATCAGCTGACA-3′ (SEQ ID NO:772), 2^{nd} round sense primer 3a.core.F2 5′-TCAAAGAAAAACCAAAAGAAACACCATCCG-3′ (SEQ ID NO:773) and 2^{nd} round antisense primer PCR3a.NS3-R2V2 5′-TTACTTCCAGATCAGCTGACA-3′(SEQ ID NO:774). PCR was performed in MicroAmp 96-well reaction plates (Applied Biosystems, Foster City, Calif.) with the following PCR parameters: 1 cycle of 94° C. for 2 min; 35 cycles of a denaturing step of 94° C. for 15 s, an annealing step of 58° C. for 30 s, an extension step of 68° C. for 5 min, followed by a final extension of 68° C. for 10 min. The product of the 1^{st} round PCR was subsequently used as a template in the 2^{nd} round PCR under same conditions but with a total of 45 cycles. Amplicons were inspected on precasted 1% agarose E-gels 96 (Invitrogen Life Technologies, Carlsbad, Calif.). All PCR procedures were carried out under PCR clean room conditions using procedural safeguards against sample contamination, including pre-aliquoting of all reagents, use of dedicated equipment, and physical separation of sample processing from pre- and post-PCR amplification steps.

For DNA sequencing, 5′ half-genome amplicons were directly sequenced by cycle-sequencing using BigDye terminator chemistry and protocols recommended by the manufacturer (Applied Biosystems; Foster City, Calif.). Sequencing reaction products were analyzed with an ABI 3730×1 genetic analyzer (Applied Biosystems; Foster City, Calif.). Both DNA strands were sequenced using partially overlapping fragments. Individual sequence fragments for each amplicon were assembled and edited using the Sequencher program 4.7 (Gene Codes; Ann Arbor, Mich.). Inspection of individual chromatograms allowed for the identification of amplicons derived from single versus multiple templates. The absence of mixed bases at each nucleotide position throughout the entire 5′ half-genome sequences was taken as evidence of single genome amplification from a single viral RNA/cDNA template. This quality control measure enabled us to exclude from the analysis amplicons that resulted from PCR-generated in vitro recombination events or Taq polymerase errors and to obtain multiple individual 5′ half-genome, core and env sequences that proportionately represented those circulating in vivo in HCV virions (SEQ ID NOS: 23-757)).

All sequence alignments were initially made with GeneCutter (www.hiv.lanl.gov) to compensate for frame shifting mutations. Because the alignment was large and the env genes riddled with insertions and deletions, and because automatic multiple sequence alignment programs are often not effective in hypervariable regions, an iterative alignment process was developed to hand-check and improve the alignments. A consensus sequence for the sequence set from each individual was generated, which was then extracted from the full alignment and hand adjusted to improve the alignment. The within patient sets were then realigned to each patient consensus, each within patient alignment again hand adjusted, and a new consensus for each patient generated. This process was iterated several times to improve the alignments. To generate the final consensus sequence for each patient, ties near regions of insertion and deletions were resolved by considering the proximal codons and context. The full alignment is available in a supplemental data file, and the sequences are also available through GenBank. All 900 5′ half-genome sequences from acute and chronic patients and were deposited in GenBank and edited alignments can be accessed at www.hiv.lanl.gov/content/sequence/hiv/user_alignments/xxxx.

### Example 4

### Phylogenetic Analysis: Identification and Enumeration of Transmitted Viruses

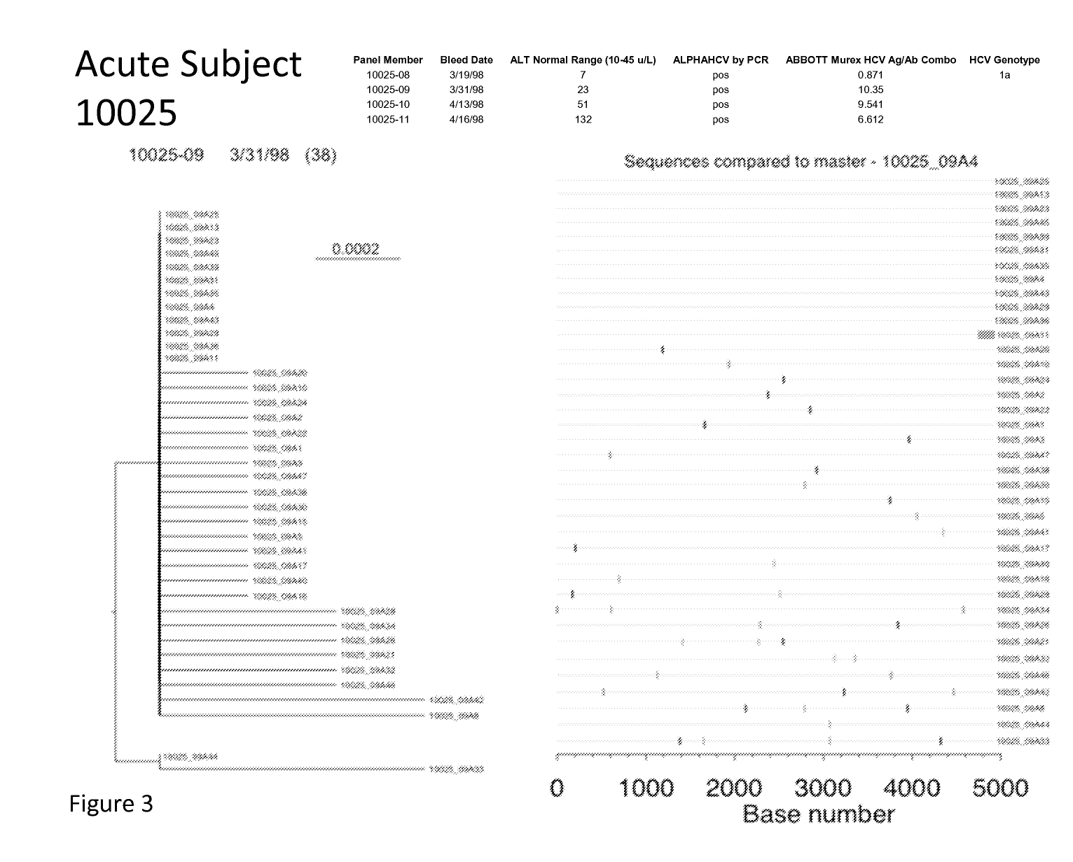

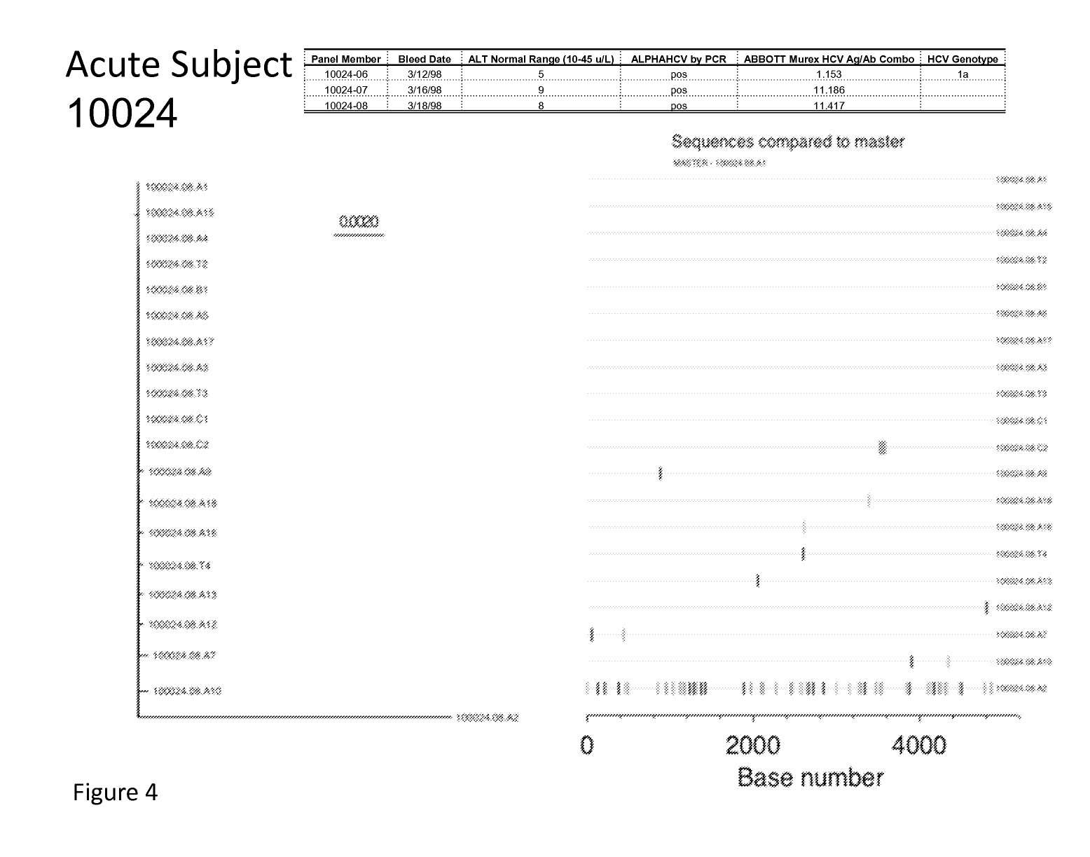

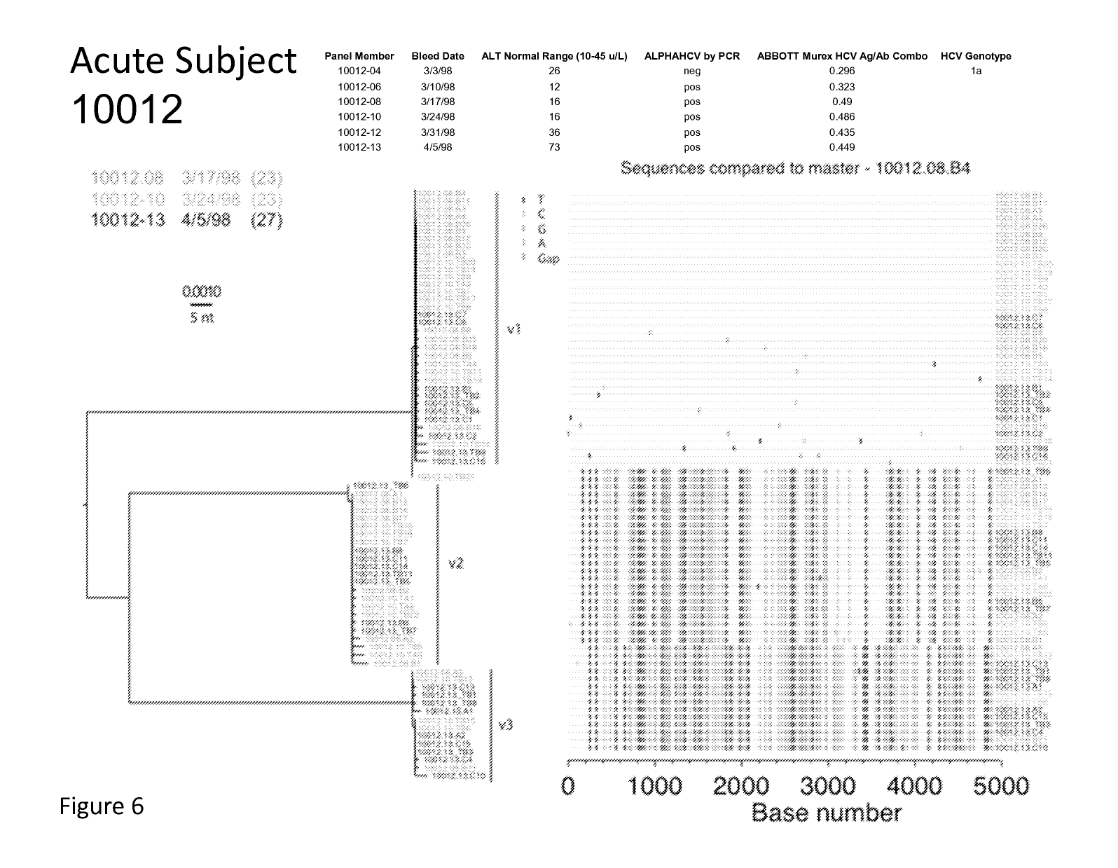

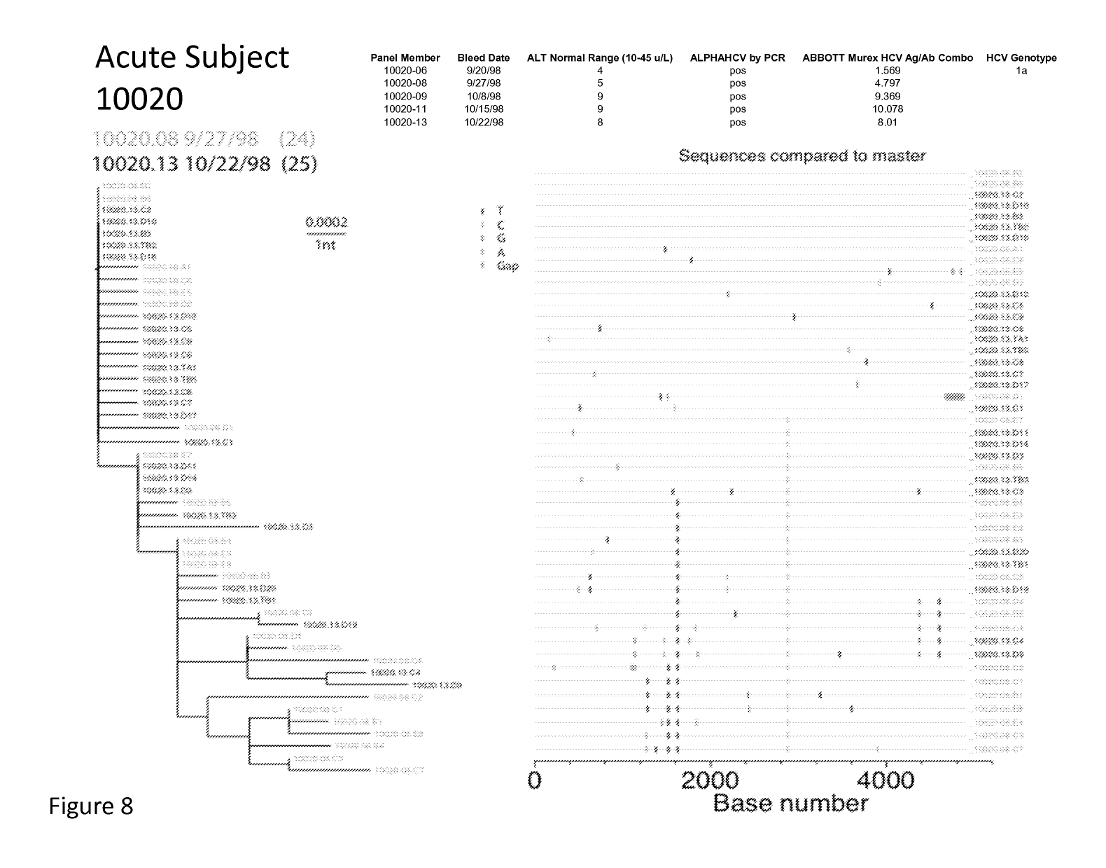

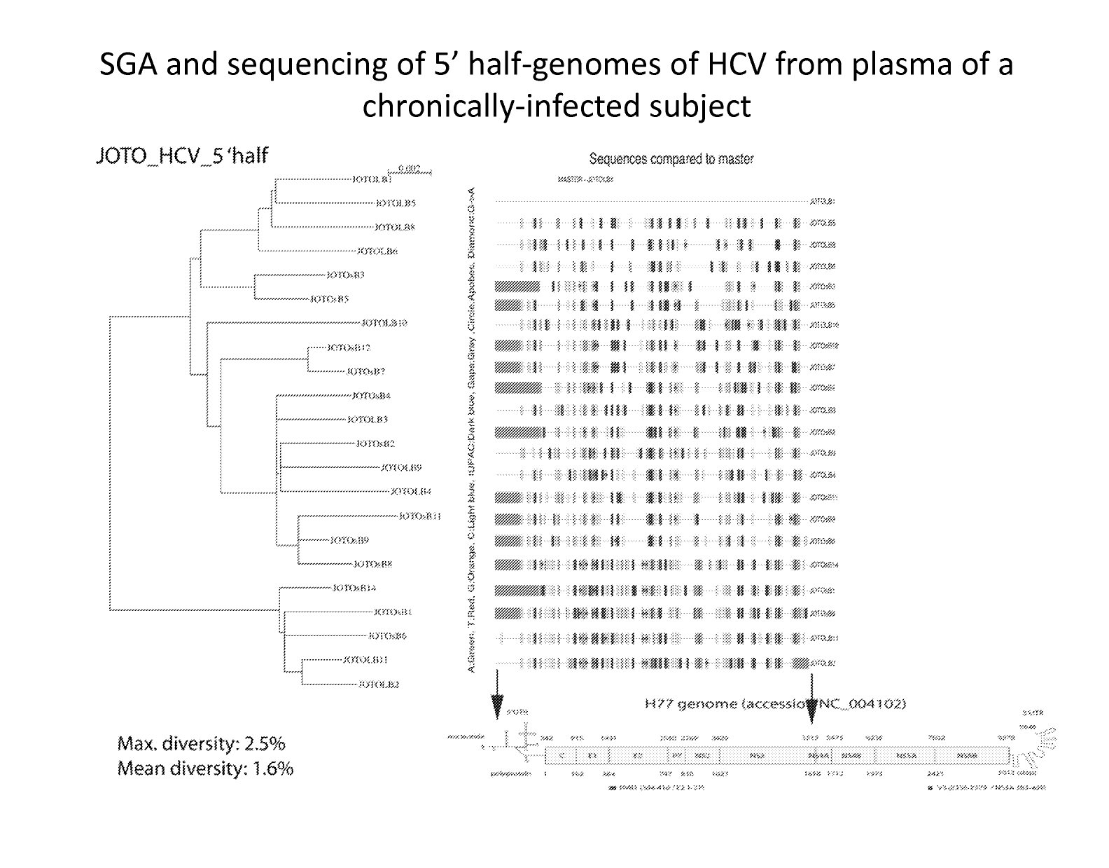

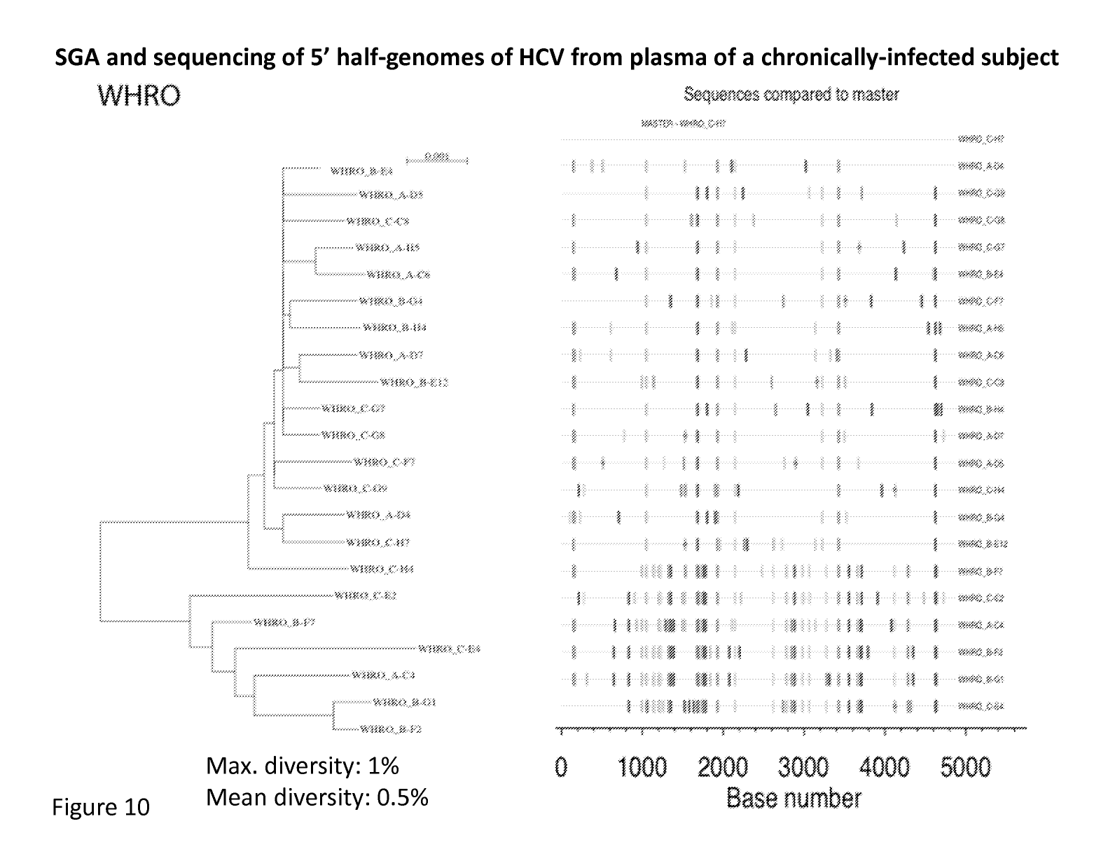

Sequences from all 10 acutely-infected subjects were analyzed using neighbor-joining (NJ) phylogenetic tree methods together with a sequence visualization tool, Highlighter (www.HIV.lanl.gov), which allows tracing of common ancestry between sequences based on individual nucleotide polymorphisms. In all 10 subjects, we identified one or more distinct, low diversity monophyletic core, env and 5′ half-genome lineages. Examples are shown in FIGS. 3-8, which are to be compared with sequences from chronically-infected control subjects (FIGS. 9 and 10). Each lineage contained a unique set of identical or near identical sequences. Three of ten subjects with more homogeneous sequences had sequences that formed single lineages in NJ trees. One other subject had sequences that exhibited low overall diversity but were comprised of 4 distinct lineages distinguished by sets of 3 or 4 nucleotide polymorphisms. Model projections suggested that these subjects were infected by very closely related viruses, most likely from a source who himself or herself was recently infected, as opposed to a single virus that evolved into two distinct lineages in the brief period preceding peak viremia. Among the subjects with greater sequence diversity, all had sequences represented by two to twelve discernible lineages. There was no evidence of inter-lineage recombination. From the combined NJ and Highlighter analyses and modeling, we concluded that 3 of the 10 subjects (30%) had been productively infected by a single virus and 7 others (70%) had been infected by at least two to twelve infectious units.

The observed differences in maximum sequence diversity could be explained by differences in the numbers of viruses that infected these individuals, and this was examined by comparing model estimates for each subject (analyzed individually) for the minimum number of days that would be required to explain the observed within-patient sequence diversification from a single most recent common ancestor (MRCA) sequence. In this model, we do not adjust for mutations that are selected against and go unobserved because they result in unfit viruses; as a consequence, the timing estimates based on a comparison of the observed data to the model would tend to be biased towards a low estimate. Each of 6 subjects with more diverse viral sequences had minimum estimates for days since a MRCA virus that exceeded plausible values given their acute infection status. Conversely, all 4 subjects with more homogeneous sequences had sequence diversities and estimated days since a MRCA that fell well within or near model predictions, suggesting that these individuals had productive clinical infections originating from a single virus or from more than one very closely related virus.

To explore how sequences sampled during viral ramp-up in the pre-antibody seroconversion period conformed to model assumptions, sequences from the 4 subjects with low diversity were examined. For each subject, the frequency distribution of all intersequence Hamming distances (HD, defined as the number of base positions at which two genomes differ) and determined if it deviated from a Poisson model using a chi-squared goodness of fit test was obtained. Next it was determined whether or not the observed sequences evolved under a star-phylogeny model (i.e., all evolving sequences are equally likely and all coalesce at the founder) in the expected time frame based on clinical stage. Three of four samples were consistent with both the Poisson model and a star phylogeny. Among the samples that deviated in their mutational patterns from a Poisson distribution, it was found one samples had apparent branching structures based on sublineages with a small number of shared mutations. Based on the temporal appearance and patterns of these mutations, it is indicated that these sublineages are resulting from transmission of closely related variants of a donor quasispecies, or from stochastic mutations generated shortly after transmission, or from HLA-restricted CTL escape mutations that accumulated in patients sampled at later time-points. Sequences from subjects with heterogeneous sequences resulting from infection by more than one virus violated model expectations for Poisson distribution and star phylogeny of mutations but conformed when identifiable core, env and 5′ half-genome sub-lineages were analyzed individually.

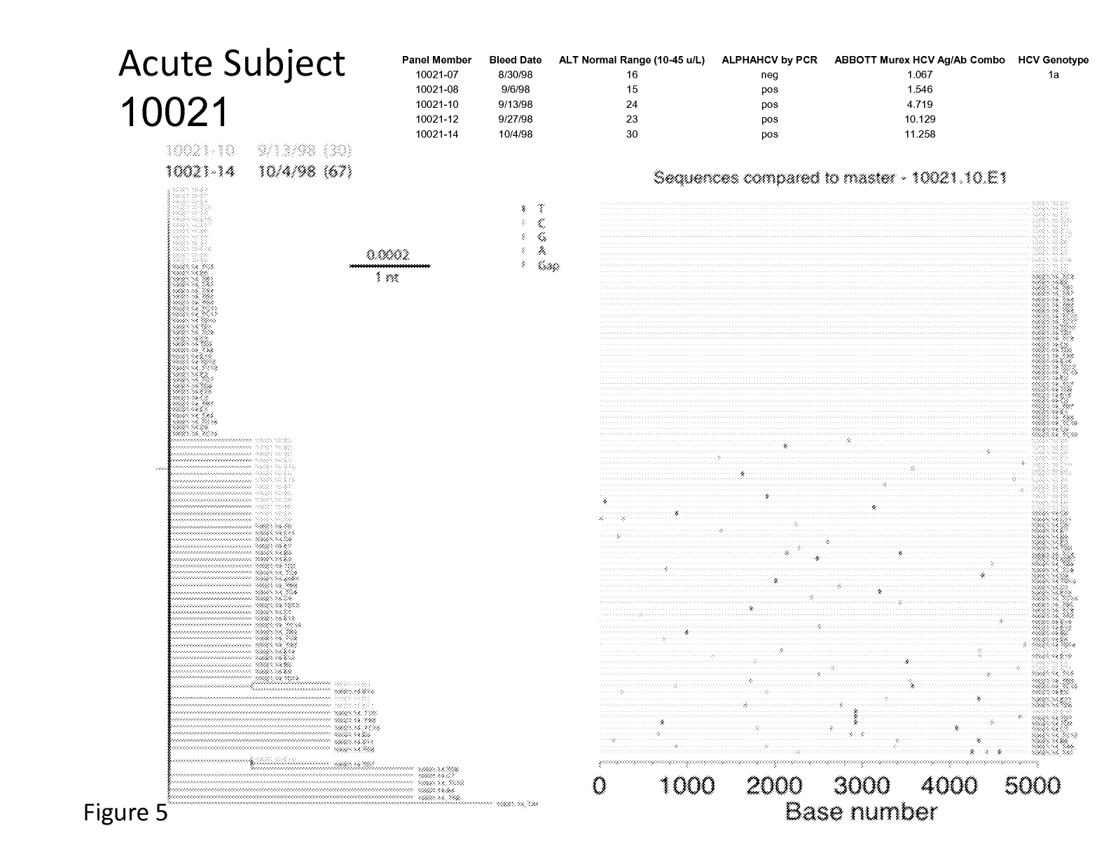

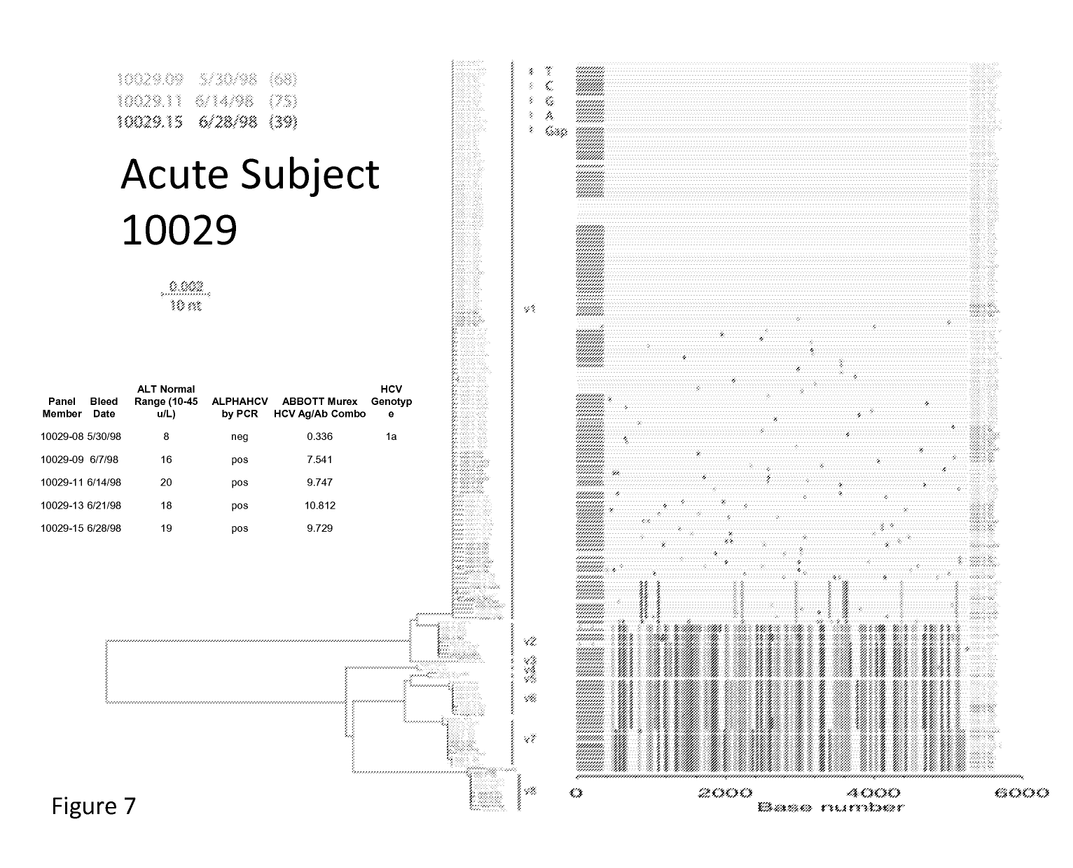

Virus diversification was also examined directly by studying four subjects sampled longitudinally. This analysis included a total of 436 5′ half-genome sequences (ranged from 65-201 aa). One subjects had evidence of infection by one virus and three subjects by more than one virus. The model assumes that before the onset of immune selection, virus evolves randomly with the proportion of sequences identical to the transmitted virus(es) declining with time and sequential rounds of virus infection and replication. For subject 10029, (FIG. 7), whose plasma was sampled three times over a period of 21 days, the proportion of identical viral sequences of variant 1 declined from 46% to 43% to 15%, consistent with model projections. In all four subjects, (FIG. 5-8), it was found that the proportion of identical half-genome sequences declined in a manner that closely approximated the model. Importantly, in none of these 4 individuals was there evidence of a transmitted virus lineage that was lost during the acute infection period, nor evidence of a predominant viral lineage that appeared subsequently.

While the empirical results suggested that HCV sequences sampled during ramp-up viremia coalesce to virus(es) at or near transmission, alternative explanations were considered. Limitations imposed by virus sampling was considered. With a sample of at least 30 plasma vRNA sequences, this permitted 95% confident that a given missed variant comprised less than 10% of the virus population (Keele, B. F. et al., 2008, Proc Natl Acad Sci USA 105:7552-7557). Sampling biases were further minimized by sequential analyses in subjects and by additional SGAs using different primer sets, all of which gave identical results. Thus, sampling biases are unlikely to affect the conclusions regarding the minimum number or identity of the viral lineages that established productive clinical infection.

The possibility that sequences sampled during ramp-up viremia might not coalesce to a transmitted or founder virus but instead to a more recent common ancestor that evolved from this virus was also considered. Several lines of evidence demonstrated this was not the case. First, in all subjects, the estimated time to the MRCA (±95% C.I.) of sequences analyzed by Poisson and BEAST models overlapped the estimated durations of infection based on clinical history. Second, the frequency of HCV RNA polymerase-mediated nucleotide misincorporation affecting a half-genome length segment (˜5000 bp) in a single infection cycle is small (2×10⁻⁵×5000=0.10, or 1 half-genome mutation in every 10 virus infection events), and most mutations would be expected to be neutral or deleterious. Evidence for the latter was found by a statistically significant trend for lower than expected dN/dS ratios in half-genome of viruses from (p<0.01 by Wilcoxon signed rank test with continuity correction). Third, the relatively long HCV generation time (1-10 days), low R₀ (<10), and brief eclipse period (˜14-21 days) provided little time or opportunity for generation and outgrowth of a selected variant, a conclusion supported by model projections (SI). Finally, SGA was used to obtain >300 5′ half-genome sequences from 10 chronically infected, treatment naïve subjects. Maximum within-subject sequence diversity among the chronic subjects ranged from 1-4%, essentially overlapping the range of HDs found in the samples from the 6 of 7 acute patients infected by more than one divergent virus. None of the chronically infected subjects exhibited predominant low diversity lineages comparable to those found in acutely infected subjects. In conclusion, the 7 subjects in whom discrete low diversity viral sequence lineages were identified, these lineages most often coalesce to sequences of actual transmitted viruses. Consensus sequences of transmitted viruses identified by the method of the invention are provided in Table I.

The identification of viral core and env genes responsible for productive clinical infection will permit the examination of the phenotypic properties of core and Env proteins most relevant to natural virus infection. This is accomplished by expression analyses and recombinant DNA chimeric virus construction. Additionally, full-length transmitted HCV genomes are readily identified by SGA sequencing methods coupled with the mathematical phylogenetic analysis disclosed herein.

The current studies identified the genetic properties of HCV at and near the moment of transmission and in the critical period of virus replication and diversification leading to peak viremia and antibody seroconversion. Among 10 subjects, we found 3 (30%) to have evidence of infection by a single virus or virus-infected cell and 7 others (70%) by at least 2 to 12 viruses. Aside from early selection of CTL escape variants found in several subjects, there was no suggestion of virus adaptation to a more replicative variant or bottlenecking in virus diversity preceding peak viremia. These findings regarding the number of viruses leading to productive clinical infection are minimal estimates, and additional viruses could conceivably have been transmitted but not sufficiently propagated in vivo to allow detection within the scope or timing of our sampling. We note, however, the observation of a low number of transmitted viruses (range 1-12; median 3) is consistent with epidemiological observations of the relative inefficiency of virus transmission by most routes (Tohme, R. A. et al., 2010, Heptalogy, (pre-print Epub:DOI:10.1002/hep.23808); Liu, C. H. et al., 2006, Clin Infect Dis, 42:1254-1259; Kleinman, S. H. et al., 2009, Transfusion, 49:2454-2489). The observed findings of low multiplicity infection and limited viral evolution preceding peak viremia suggest a crucial but finite window of potential vulnerability of HCV to potential treatments including vaccine-elicited immune responses.

### Example 5

### Methods of Phylogenetic Analysis and Cloning of HCV Genomes

5′ half-genome, core and env diversity analysis was performed as follows. We classified two very distinctive levels of within-patient sequence diversity that we observed in the 10 study subjects as either “homogeneous” or “heterogeneous.” This was done using three different strategies that all concurred. Firstly, samples were visually inspected using neighbor-joining phylogenies and the Highlighter tool (www.hiv.lanl.gov) and it was found that 6 samples clearly had much greater diversity than 4 others. Next, all pairwise Hamming Distances were examined (HD, defined as the number of base positions at which the two genomes differ, excluding gaps) within each sample. The same 6 heterogeneous samples exhibited distinct peaks with a multimodal distribution inconsistent with expansion from single infecting virus. Lastly, to formalize the criteria and test whether the 6 heterogeneous samples reflected transmission of multiple variants, a mathematical model described herein was used to predict the expected maximum HD that could be observed under a homogeneous infection assumption (i.e., infection by a single virus), given the clinical stage of the sample. If the maximum HD in the sample was much greater than the expected, the observed diversity was considered to have originated at a time prior to transmission, i.e. in the donor indicating that multiple strains transmitted from the donor to the recipient established the infection; this was again the case for all 6 heterogeneous samples. For the homogeneous samples, we considered the possibility that these individuals had been infected by a single virus (or infected cell) or by two or more very closely related viruses. Either scenario could result in a low overall sequence diversity, but in the case of transmission of two or more very closely related viruses, the distribution of HDs would not fit model expectations. This was observed to be the case in 1 of the 4 subjects with homogeneous infections.

Star phylogeny analysis was performed as described. With no selection pressure, one can expect homogeneous viral populations to evolve from a founder strain following a star-like phylogeny, (i.e., all evolving sequences coalesce at the founder). The veracity of this proposition can be investigated by inspecting the sequence alignment. Because mutations are rare, one does not expect shared mutations in a star phylogeny. When this is indeed the case, the distribution of intersequence HD's is constrained to be a self-convolution (defined below) of the distribution of the HD's from the sequences to the ancestral sequence. In particular, for every pair of sequences s₁ and s₂, let HD[s₁,s₂] be the number of base positions at which the two differ and the probability distribution it follows be P_{I}(HD). Next, each sequence in the sample is compared with the consensus sequence, which is assumed to be the founder strain, followed by computation of the corresponding HD distribution. Denoting s₀ the founder strain, for every sequence s₁ computed HD[s₀,s₁] and denoted P_{c}(HD) the distribution it follows. Under a star-phylogeny evolution, P_{I}(HD) is given by the self-convolution of P_{C}(HD):

$${P}_{I} ( HD = n ) = {\sum }_{k = 0}^{n} {P}_{C} ( HD = k ) {P}_{C} ( HD = n - k )$$

Occasional deviations from a star phylogeny are, however, expected. The sampling of 30 sequences, for example, from a later generation of an exponentially growing population with six-fold growth per generation has about 5% chance of including a pair of sequences, which shares five initial generations, a 25% chance of those sharing the first four, and overwhelmingly likely to include sequences that share three ancestors. However, because the rate of mutations in the region under study is about 1 per 20 generations (see next section), this leads to only about 10% chance of finding sequences sharing a pair of mutations, and less than 1% chance of sharing more than that. The probabilities are slightly enhanced by the early stochastic events that can lead to the virus producing less than six descendants in some generations, but it remains overwhelmingly likely that the sequences share few mutations. Thus when a sample had two or more sublineages of sequences that were defined by more than two shared mutations, the observation is best explained by transmission of multiple closely related viruses (3 such cases were identified). In later clinical stages, CTL driven immune selection might contribute to such a pattern and selection cannot be distinguished from transmission of multiple viruses.

In the mathematical models described in Example 1, it is assumed a homogeneous infection occurs in which the virus grows exponentially with no selection pressure, no recombination, no occurrence of back mutations and a constant mutation rate across positions and across lineages. Under this scenario, the HD frequency distribution is given by a Poisson distribution whose mean depends linearly on the number of generations since the founder strain. Previously estimated parameters of HIV-1 generation time (2 days) (Markowitz, M. et al., 2003, J Virol, 77:5037-5038), reproductive ratio (R₀, 6) (Stafford, M. A. et al., 2000, J Theor Biol, 203:285-301), reverse transcriptase point mutation rate (ε=2.16×10⁻⁵) (Mansky, L. M. et al., 1995, J Virol, 69:5087-5094) were utilized as starting point. It was further assumed that the initial virus replicated exponentially by infecting exactly R0 new cells at each generation, which, for simplicity, happened in two equal bursts at τ and 2τ. The reverse transcriptase error rate estimate (Mansky, L. M. et al., 1995, J Virol, 69:5087-5094) is based on sequencing virus produced in vitro after a single round of replication. If a mutation occurs that is lethal with regard to viral production it would not be detected in this assay, and such mutations may be similarly reduced in the natural, in vivo situation. On the other hand, lethal mutations that were not infectious would be retained in the single round of replication assay, but may be selected against in vivo, hence the mutation rate we are using in the model will have a bias towards being greater than the substitution rate we observe in vivo, potentially resulting in slight underestimates of the time to the MRCA.

The intersequence HD's are not independent, but because of the star phylogeny they are the pairwise sums of a set of independent Poisson distributed variates. The form of their distribution, including the (singular) covariance matrix, is therefore known up to one unknown parameter, the lambda of the underlying Poisson distribution. This parameter was estimated by fitting the observed data to the expected form using a Maximum Likelihood method, and assessed the goodness of fit using a chi-square goodness-of fit test statistic calculated from a singular value decomposition of the covariance matrix. When the data were consistent with a Poisson distribution, we used the λ of the best fitting distribution to estimate a divergence time from the most recent common ancestor (MRCA) based on the estimated number of generations required to achieve the observed distribution. One can in fact show the following relationship for λ:

$$λ ( t ) = ɛ {N}_{B} ( \frac{5}{8} t \frac{1 + ϕ}{ϕ} + \frac{1 - ϕ}{{ϕ}^{2}} )$$

Therefore, once we obtain a best fitting Poisson distribution, we calculate its mean λ* and use the above time-dependency relationship to estimate time since MRCA (in days) as follows:

$$t = \frac{8 ϕ}{5 ( 1 + ϕ )} ( \frac{{λ}^{*}}{ɛ {N}_{B}} - \frac{1 - ϕ}{{ϕ}^{2}} )$$

where N_{B} is the sequence length in base pairs and

$$ϕ = \sqrt{1 + \frac{8}{{R}_{0}}} .$$

Furthermore, the fraction of identical sequences expected at that time is:

$$Exp ⌊ - ɛ {N}_{B} ( \frac{5}{8} t \frac{1 + ϕ}{ϕ} + \frac{1 - ϕ}{{ϕ}^{2}} ) + O ( {ɛ}^{2} {N}_{B} ) ⌋$$

The change in the Poisson distribution over time illustrates the increasing diversity expected under the model (SI FIG. 13). It is apparent that as time increases, the number of identical sequences decreases and the frequency distribution of the intersequence HD's at various times post-infection shifts to higher HD values.

Bayesian analysis was performed as follows. The time, in days, to the most recent common ancestor (MRCA) for each patient was also estimated using a Bayesian Markov Chain Monte Carlo (MCMC) approach, implemented in BEAST v1.4.1 (Drummond, A. J. et al., 2006, PLoS Biol 4:e88; Drummond, A. J. et al., 2007, BMC Evol Biol 7:214). The mean substitution rate was fixed at 2.16×10⁻⁵ substitutions per site per generation and all analysis were carried out using the General Time Reversible (GTR) substitution model with invariant sites and gamma-distributed rate heterogeneity (4 gamma categories). The substitution and rate heterogeneity models were unlinked across codon positions and we assumed exponential population growth and a relaxed (uncorrelated exponential) molecular clock. This model was used for analysis of each patient's viral sequence alignment and the MCMC algorithm was run for at least 10⁷ (Drummond, A. J. et al., 2006, PLoS Biol, 4:e88) generations (logging every 1000 generations; and burn-in set to 10% of the original chain length), with additional runs carried out if the Effective Sample Size (ESS) for the estimate was below 100. The results were visualized in TRACER (Drummond, A. J. et al., 2005, Mol Biol Evol 22:1185-1192). We repeated this analysis with the five free parameters of the GTR model fixed at values estimated using the combined data from all acute patients inferred to be infected with a single viral strain using the HyPhy package (Kosakovsky Pond, S. L. et al., 2006, Mol Biol Evol 23:1891-1901) and with alternative demographic and evolutionary models (relaxed uncorrelated molecular clock with logistic population growth and strict molecular clock with exponential population growth). Estimates and confidence intervals for the MRCA times were similar for the alternative relaxed clock models, but approximately 25% lower using a strict molecular clock (not shown).

To better understand our likelihood of missing infrequent transmitted variants, a power study was performed to explore the probability of sampling limitations. It was shown that with a sample of at least n=20 plasma vRNA sequences, we could be 95% confident that a given missed variant comprised less than 15% of the virus population (Keele, B. F. et al., 2008, Proc Natl Acad Sci USA 105:7552-7557). For samples for which r≧30, we could be 95% confident not to have missed any variant that comprised at least 10% of the total viral population.

Core and env gene and full-length viral genome cloning can be performed as described herein. SGA-derived amplicons containing full-length core or env genes can be molecularly cloned for protein expression and biological analysis. Transmitted core and env were identified as described along with SGA-derived genes from chronically infected control subjects. In order to reduce the probability of generating molecular core and env clones with Taq polymerase errors, samples were re-amplified from the first round PCR product under the same nested PCR conditions but used 10 fewer cycles. Correctly-sized amplicons identified by gel electrophoresis were gel purified using the QIAquick gel purification kit according to manufacturer's recommendations (Qiagen, Valencia, Calif.), ligated into the pcDNA3.1 Directional Topo vector (Invitrogen Life Technologies, Carlsbad, Calif.), and transformed into TOP10 competent bacteria. Bacteria were plated on LB agar plates supplemented with 100 μg/ml of ampicillin and cultured overnight at 30° C. Single colonies were selected and grown overnight in liquid LB broth at 30° C. with 225 rpm shaking followed by plasmid isolation. Each molecular clone was sequenced confirmed to be identical to the transmitted core and env sequence(s) for each patient. Full-length genomes were chemically synthesized, as described (Salazar-Gonzalez, J. F. et al., 2009, J Exp Med 206:1273-1289) and cloned into pcDNA3.1 vectors, as described above.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

APPENDIX A
Table I: 5′Transmitted HCV Sequence: Inferred 5/ Half Transmitted/Founder Virus Sequence

Donor 68470/ Panel Number 10021/ Transmitted Variant #1 (SEQ ID NO: 1)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGTAGACGTCAGCCTATCCCCAAGGCGCGTCGGCCCGAGGGCAGGTCCTGGGCTCAGCC
CGGGTACCCTTGGCCCCTCTATGGCAATGAGGGTTGCGGGTGGGCGGGATGGCTCCTGTCTCCCCGTGGCTCTCGGCCTAGCTGGGGCCCCAC
AGACCCCCGGCGTAGGTCGCGCAATTTGGGTAAGGTCATCGATACCCTTACGTGCGGCTTCGCCGACCTCATGGGGTACATACCGCTCGTCGG
CGCCCCTCTCGGAGGCGCTGCCAGGGCCCTGGCGCATGGCGTCAGGGTTCTGGAAGACGGCGTGAACTATGCAACAGGGAATCTTCCTGGTTG
CTCTTTCTCTATCTTCCTTCTGGCTCTGCTCTCTTGCCTGACCGTGCCCGCTTCAGCCTACCAAGTGCGCAACTCTTCGGGGCTTTACCATGT
CACCAATGATTGCCCTAACTCGAGTATTGTGTACGAGGCGGCCGATGCCATTCTGCACACTCCGGGGTGTGTCCCTTGTGTTTACGAGGGTAA
CACCTCGAGGTGTTGGGTGGCGGTGACCCCCACAGTGGCCACCAGGGACGGCAAACTCCCCACGACGCAGCTTCGACGCCACATCGACCTGCT
TGTCGGGAGTGCCACCCTCTGCTCGGCCCTCTACGTGGGGGATCTGTGCGGGTCTGTCTTTCTTGTCGGCCAACTGTTCACCTTCTCTCCCAG
GCGCCACCGGACGATGCAGAACTGCAATTGTTCTATCTACCCCGGCCATATAACGGGCCACCGCATGGCATGGGATATGATGATGAACTGGTC
CCCTACGGTGGCATTGGCGGTAGCTCAGCTGCTCCGGATCCCACAAGCCATCATGGACATGATCGCTGGTGCTCACTGGGGAGTCCTGGCGGG
CATAGCGTATTTCTCCATGGTGGGGAACTGGGCGAAGGTCCTGGTAGTGCTGCTGCTATTTGCCGGCGTCGACGCGGAGACCCACGTCACCGG
GGGAGCTGCCGGCCGCTCCACGGCTGGGATTGCTGGTCTCTTCAGCCCAGGCGCCAGACAGAACATCCAACTGATCAATACCAACGGCAGTTG
GCACATCAATAGCACGGCCCTGAACTGCAACGACAGCCTCAACACCGGCTGGATAGCAGGGCTCTTCTATTCCAACAAATTCAACTCTTCGGG
CTGTCCCGAGAGGTTGGCCAGCTGCCGACGCCTTGCCGATTTTGCCCAGGGCTGGGGCCCCATCAGTTACGCCAACGGAAGCGGCCCCGACGA
ACGCCCCTACTGCTGGCACTACCCCCCAAGACCTTGTGGTATTGTGCCCGCTAAGAGCGTGTGTGGCCCGGTGTATTGCTTCACTCCCAGCCC
CGTGGTGGTGGGAACGACCGACAGGTCGGGCGCGCCTACCTACAACTGGGGTGAAAATGACACGGACGTCTTCGTCCTCAACAACACCAGGCC
GCCGCTGGGCAATTGGTTCGGTTGTACCTGGATGAACTCAAGTGGATTCACCAAAGTGTGCGGAGCGCCCCCTTGTGTCATCGGAGGGGTGGG
CAACAACACCTTGCGCTGCCCCACTGATTGCTTCCGCAAACATCCGGAAGCCACATACTCTCGGTGCGGCTCCGGTCCTTGGATCACACCCAG
GTGCATGGTCGACTACCCATATAGGCTTTGGCATTATCCTTGTACCATCAACTACACCTTATTCAAAGTCAGGATGTACGTGGGAGGGGTCGA
GCACAGGCTGGAAGCTGCCTGCAACTGGACGCGGGGCGAACGCTGTGATCTGGAAGACAGGGACAGGTCCGAGCTCAGTCCGTTACTGCTGTC
CACCACGCAGTGGCAGGTCCTTCCGTGTTCTTTTACGACCCTGCCAGCCTTGTCCACCGGCCTCATCCACCTCCACCAGAACATTGTGGACGT
GCAGTACTTGTACGGGGTAGGGTCAAGCATCGCGTCCTGGGCCATTAAGTGGGAGTACGTCGTTCTCCTGTTCCTTCTGCTTGCAGACGCGCG
CGTCTGCTCCTGCTTGTGGATGATGTTACTCATATCCCAAGCGGAGGCGGCTTTGGAGAACCTCGTAGTACTCAATGCAGCATCCCTGGCCGG
GACGCACGGTCTTGTGTCCTTCCTCGTGTTCTTCTGCTTTGCGTGGTATCTGAAGGGTAGGTGGGCGCCCGGAGCGGTCTACGCCCTCTACGG
GATGTGGCCTCTCCTCCTGCTCCTGCTGGCGCTGCCTCAGCGGGCATACGCACTGGACACGGAGGTGGCCGCGTCGTGTGGCGGCGTTGTTCT
TGTCGGGTTGATGGCGCTGACTCTGTCACCACATTACAAGCGCCTCATCTGCTGGTGCGTGTGGTGGCTTCAGTATTTTCTAACCAGAGTGGA
AGCGCACCTGCACGTGTGGGTTCCCCCCCTCAACGTCCGGGGGGGGCGCGATGCCATCATCTTACTCATGTGTGTTATACACCCGGCTCTGAT
ATTTGACATCACCAAACTGCTGCTGGCCGTCTTCGGACCCCTTTGGATTCTTCAAGCCAGTTTGCTGAAAGTCCCCTACTTCGTGCGCGTTCA
AGGCCTTCTCCGAATCTGCGCGCTAGCGCGGAAGTTAGCCGGAGGTCATTACGTGCAGATGGCCATCATCAAGTTAGGGGCGCTTACTGGCAC
CTATGTCTATAACCATCTCACTCCTCTCCGAGACTGGGCGCACAACGGCCTGCAAGACCTGGCCGTGGCTGTAGAACCAGTCATCTTCTCCCG
AATGGAGACCAAACTCATCACGTGGGGGGCAGACACCGCCGCGTGCGGCGACATCATCAACGGCTTGCCCGTCTCCGCCCGCAGGGGCCGGGA
GATATTGCTTGGACCAGCCGACGGAATGGCCTCTAAAGGGTGGAGGTTGCTGGCGCCCATCACAGCGTACGCCCAGCAGACGAGAGGCCTCCT
AGGGTGCATAATCACCAGCCTGACTGGCCGGGATAAAAACCAAGTGGAGGGTGAGGTCCAGATCGTGTCAACTGCTACCCAAACCTTCCTGGC
AACGTGCATCAATGGGGTATGCTGGACTGTCTACCACGGGGCCGGAACGAGGACCATCGCATCACCCAGGGGTCCTGTTATCCAGATGTACAC
CAATGTGGACCAGGACCTTGTGGGCTGGCCTGCTCCTCAAGGTTCCCGCTCATTGACACCCTGCACCTGCGGCTCCTCGGACCTTTACCTGGT
CACGAGGCACGCCGATGTCATTCCCGTGCGCCGGCGAGGTGATAGCAGGGGCAGCTTGCTCTCGCCCCGGCCCATTTCCTACTTGAAAGGCTC
CTCGGGGGGTCCGCTGTTGTGCCCCGCGGGACACGCCGTGGGCATATTTAGGGCCGCGGTGTGCACCCGTGGTGTGGCTAAGGCGGTGGACTT
CATCCCTGTGGAGAACCTAGAGACAACCATGAGATCCCCGGTGTTCACGGACAACTCTTCTCCACCAGCAGTGCCCCAGAGCTTCCAGGTGGC
TCACCTGCATGCTCCCACTGGCAGCGGCAAGAGCACCAAGGTCCCGGCTGCGTACGCAGCTCAGGGCTACAAAGTGCTAGTGCTCAACCCCTC
TGTTGCTGCAACATTGGGCTTTGGTGCTTACATGTCCAAGGCTCATGGGGTCGATCCTAATGTCAGGACGGGGGTGCGAACAATTACCACCGG
CAGCCCCATCACGTACTCCACCTACGGCAAGTTCCTTGCCGACGGCGGGTGCTCAGGAGGTGCTTATGACATAATAATTTGTGATGAGTGCCA
CTCCACGGATGCCACATCCATCTTGGGCATCGGCACTGTCCTTGACCAAGCAGAGACCGCGGGGGCGAGACTGGTTGTGCTCGCCACTGCTAC
CCCTCCGGGCTCCGTCACCGTGCCCCATCCTAACATCGAGGAGGTTGCTCTGTCCACCACCGGAGAGATCCCTTTTTACGGCAAGGCTATTCC
CCTCGAGGTCATCAAGGGGGGAAGGCATCTCATCTTCTGCCACTCAAAGAAAAAGTGTGACGAGCTCGCCGCAAAGCTGGTCGCATTGGGCAT
CAATGCCGTGGCCTACTACCGCGGTCTTGACGTGTCTGTCATCCCGACCAGCGGCGATGTTGTCGTCGTGGCGACCGATGCTCTCATGACTGG
CTATACCGGCGACTTCGACTCTGTGATAGACTGCAACACGTGTGTCACCCAGACAGTCGATTTCAGCCTTGACCCTACCTTCACCATTGAGAC
AACCACGCTCCCCCAAGACGCTGTCTCCCGGACTCAACGTCGGGGCAGAACCGGCAGGGGGAAGCCAGGCATCTACAGATTTGTGGCACCGGG
GGAACGCCCCTCCGGCATGTTCGACTCGTCCGTCATCTGTGAGTGCTATGACGCGGGCTGTGCTTGGTATGAGCTCACGCCCGCCGAGACTAC
AGTTAGGCTACGAGCGTACATGAACACCCCGGGGCTCCCCGTGTGCCAGGACCATCTTGAATTTTGGGAGGGCGTCTTTACGGGCCTCACCCA
TATAGATGCCCACTTTCTATCCCAGACAAAGCAGAGTGGAGAGAACTTCCCTTACCTGGTAGCGTACCAAGCCACCGTGTGCGCTAGGGCTCA
AGCCCCTCCCCCATCGTGGGACCAGATGTGGAAGTGTTTGATCCGCCTCAAACCCACCCTCCATGGGCCAACACCCCTGCTATACAGACTGGG
CGCTGTTCAGAATGAAGTCACCCTGACGCACCCAGTCACCAAATACATCATGACATGCATGTCGGCTGA
Donor 66801/ Panel Number 10025/ Transmitted Variant #1 (SEQ ID NO: 2)
TCGCCCACAGGACGTCAAGTTCCCGGGTGGCGGTCAGATCGTTGGTGGAGTTTACTTGTTACCGCGCAGGGGCCCTAGATTGGGTGTGCGCGC
GACGAGGAAGACTTCCGAGCGGTCGCAACCTCGAGGTAGACGTCAGCCTATCCCCAAGGCGCGTCGGCCCGAGGGCAGGACCTGGGCTCAGCC
CGGGTACCCTTGGCCCCTCTATGGCAATGAGGGCTGCGGGTGGGCGGGATGGCTCCTGTCCCCCCGTGGCTCTCGGCCTAGCTGGGGCCCTAC
AGACCCCCGGCGTAGGTCGCGCAATTTGGGTAAGGTCATCGATACCCTCACGTGCGGCTTCGCCGACCTCATGGGGTACATTCCGCTCGTCGG
CGCGCCTCTTGGAGGCGCCGCCAGGGCCCTGGCGCATGGTGTCCGGGTTCTGGAGGACGGCGTGAACTATGCAACAGGGAACCTTCCTGGTTG
CTCTTTCTCTATCTTCCTTTTGGCCCTGCTCTCTTGCCTGACTGTACCCGCGTCGGCCTATCAAGTTCGCAACTCCTCGGGGCTTTATCATGT
CACCAATGATTGCCCTAACTCGAGTATTGTGTACGAGACGGCCGACGCCATTCTACACTCTCCGGGGTGTGTCCCTTGCGTCCGCGAGGGTAA
CGCCTCGAAGTGCTGGGTGCCGGTGGCCCCCACAGTGGCCACCAAGGACGGCAAACTCCCCACGACGCAGCTTCGACGTCACATCGATCTGCT
TGTCGGGAGCGCCACCCTCTGCTCGGCCCTCTATGTGGGGGACTTGTGCGGGTCTGTCTTCCTTGTCGGTCAACTGTTCACTTTCTCCCCCAG
ACGCCACTGGACAACGCAAGACTGCAACTGTTCCATCTACCCCGGCCATATAACGGGCCACCGCATGGCATGGGATATGATGATGAATTGGTC
TCCTACAGCAGCGCTGGTAATGGCTCAACTGCTCAGAGTCCCGCAAGCTGTCATGGACATGATCGCTGGAGCCCACTGGGGAGTCCTAGCGGG
CATAGCGTATTTCTCCATGGTGGGGAACTGGGCAAAGGTCTTGTTGGTGCTGTTGTTGTTTGCCGGCGTCGACGCGAGCACTCGCACCATCGG
GGGGTCTGCTGCTGCGACCACGTCCGGATTCGCCAAGCTCTTCGCTGCTGGCTCCCGCCAGAATGTCCAGCTGATCAACACCAACGGAAGTTG
GCACATCAATCGCACGGCCTTGAACTGTAATGCGAGTCTCGACACTGGCTGGGTAGCGGGGCTCTTCTATTACCACAAATTCAACTCTTCGGG
CTGCCCCGAGAGGATGGCCAGCTGTAGACCCCTTGCCGATTTCGACCAGGGCTGGGGCCCTATCAGCTACGCCAACGCAAGCGGCACCGACCA
CCCCCCCTACTGCTGGCACTACCCCCCAAAGCCTTGCGGTATCGTGCCGGCACAGAACGTATGTGGCCCAGTATATTGCTTCACTCCCAGCCC
TGTGGTGGTGGGAACGACCGACAAGTTGGGCGTGCCCACCTACAACTGGGGTAGCAATGACACGGACGTCTTCGTCCTTAACAACACCAGGCC
ACCGTTGGGCAATTGGTTCGGTTGCACCTGGATGGACTCATATGGATATACCAAAGTGTGCGGAGCGCCCCCTTGTGTCATCGGAGGGGTAGG
CAATAACACCTTGCACTGCCCCACTGACTGTTTCCGCAAGCATCCAGAAGCCACATACTCTCGGTGTGGCTCCGGTCCCTGGATCACGCCCAG
GTGCCTGGTCCACTACTCTTACAGGCTTTGGCATTATCCTTGTACTGTCAACTACACCCTGTTCAAAGTCAGGATGTACGTGGGAGGGGTCGA
GCACAGGCTGGAGGTCGCTTGCAACTGGACGCGGGGCGAACGTTGCGATCTGGACGACAGGGACAGGTCCGAGCTCAGCCCGCTGCTGCTGTC
CACCACACAGTGGCAGGTTCTTCCGTGTTCTTTCACGACCTTGCCAGCCTTGACTACCGGCCTCATCCACCTCCACCAGAACATCGTGGATGT
GCAATATTTGTACGGGGTGGGGTCAAGCATTGTGTCCTGGGCCATCAAGTGGGAGTACGTCATCCTCTTGTTTCTCCTGCTTGCAGACGCGCG
CATTTGTTCCTGCTTGTGGATGATGCTACTCATATCCCAGGTGGAGGCAGCTTTGGAGAACCTCGTGCTGCTCAACGCGGCGTCTCTGACCGG
GGCGCACGGTCTTGTGTCCTTCCTCGTGTTTTTCTGCTTTGCATGGTATCTGAAGGGTAGGTGGGTGCCCGGAGCGGCCTACGCCCTCTACGG
GATGTGGCCTCTCCTCCTGCTTCTGTTGGCGTTGCCCCAACGGGCATACGCACTAGACACGGAAGTGGCCGCGTCGTGTGGTGGCGTTGTTAT
TGTCGGGTTAATGGTGCTGACTCTGTCACCGCACTACAAGCGCTATATCAGCTGGTGCTTATGGTGGCTCCAATATTTCCTGACTAGAGTGGA
AGCGCAACTGCACGTATGGGTCCCCCCCCTCAACGTCCGAGGAGGCCGCGACGCTGTCATCTTGCTCATGTGTGTTATACACCCGGCCTTGGT
ATTTGACATCACCAAGCTGCTGCTGGCCGTCTTCGGACCCCTTTGGATTCTTCAAACCAGTCTGCTCAAAGTGCCCTACTTCGTGCGCGTTCA
GGGCCTTCTCCGGCTCTGCGCGCTAGCGCGCAAGATGGCCGGAGGCCATTACGTGCAAATGGCCATCATCAAGGTGGGGGCGCTTACTGGCAC
CTACATCTATAACCATCTTACTCCTCTTCGGGATTGGGCGCACAACAGCCTACGAGATCTGGCCGTGGCTGTAGAGCCAGTCGTCTTTTCCCA
GATGGAGACTAAGCTCATCACCTGGGGGGCGGACACCGCCGCCTGCGGTGACATCATCAACGGCTTGCCCGTCTCTGCCCGTAGGGGCCGAGA
GATACTGCTCGGACCGGCCGACGGAGCGGTCTCCAAGGGGTGGAGGTTGCTAGCGCCCATCACGGCGTATGCCCAGCAGACAAGGGGCCTTTT
GGGATGTATAATTACCAGCCTGACCGGCCGGGACAAAAACCAGGTGGAGGGTGAGGTTCAGATTGTGTCTACTGCTGCCCAGACTTTCCTGGC
AACCTGCATCAACGGGGTGTGCTGGACTGTCTACCACGGGGCCGGGACAAGGACCATCGCGTCGCCCAAGGGTCCCGTTATCCAGATGTATAC
CAATGTAGACCAAGACCTCGTAGGCTGGCCCGCTCCCTATGGTGCTCGCTCATTGACACCCTGCACTTGCGGCTCCTCGGACCTTTACCTGGT
CACGAGGCACGCCGATGTCATTCCCGTACGCCGGCGGGGTGATAGCAGGGGCAGCCTGCTTTCGCCTCGACCCATCTCTTACTTGAAAGGCTC
CTCGGGGGGCCCACTACTGTGCCCCGCGGGACACGCCGTAGGCATATTCAGGGCCGCGGTGTGCACCCGTGGAGTGGCTAAGGCGGTGGACTT
TATCCCCGTAGAGAGCCTAGAGACAACCATGAGGTCCCCGGTGTTCACAGACAACTCCTCCCCACCAGCGGTGCCCCAGAGCTTCCAGGTGGC
CCACCTGCACGCTCCCACCGGCAGCGGTAAGAGCACCAAGGTCCCGGCCGCATACGCGGCTCAGGGCTACAAGGTGCTGGTGCTCAACCCCTC
CGTTGCTGCAACACTGGGCTTTGGCGCCTACATGTCCAAGGCCCATGGGATTGATCCGAACATCAGGACTGGGGTGAGGACAATTACTACTGG
CAGCCCCATCACGTACTCCACCTACGGCAAGTTCCTCGCTGACGGCGGGTGCTCAGGGGGTGCTTATGACATAATAATTTGTGACGAGTGCCA
CTCCACGGATGCAACATCCATCTTGGGCATCGGCACTGTCCTTGACCAAGCAGAGACAGCGGGGGCGAGGCTGGTTGTGCTCGCCACCGCTAC
CCCTCCGGGCTCCGTCACTGTGCCCCACTCTAACATCGAGGAGGTTGCTCTGTCCACTACCGGAGAGATCCCCTTTTACGGCAAGGCTATCCC
CCTTGAGGCAATCAAGGGGGGGAGACATCTCATTTTCTGCCACTCAAGGAAAAAGTGCGACGAGCTCGCCGCAAGGCTGGTCGCGTTGGGCAT
CAATGCTGTGGCCTACTACCGCGGCCTTGACGTGTCCGTCATCCCGACCAGCGGCGACGTTGTCGTCGTGGCAACTGATGCCCTCATGACCGG
CTTTTCCGGCGACTTCGACTCGGTGATAGACTGCAACACGTGTGTCACCCAGACAGTCGACTTCAGCCTTGACCCTACCTTCACCATTGAGAC
AACCACGCTTCCTCAGGACGCTGTTTCCCGCACCCAACGTCGGGGCAGGACTGGCAGGGGGAAGCCAGGCATCTATAGATTTGTGGCACCGGG
AGAGCGCCCCTCCGGCATGTTCGACTCGTCCGTCCTCTGTGAGTGCTATGACGCAGGCTGTGCTTGGTATGAGCTCACACCCGCCGAGACCAC
AGTTAGGTTACGAGCGTACATGAACACCCCGGGGCTTCCCGTGTGCCAGGACCATCTTGAATTTTGGGAGGGCGTCTTCACAGGTCTCACCCA
TATAGACGCCCACTTCTTATCTCAGACAAAGCAGAGTGGGGAAAACTTTCCTTACCTGGTAGCGTACCAAGCCACCGTGTGCGCTAGAGCTCA
AGCTCCCCCCCCATCGTGGGACCAGATGTGGAAGTGCTTGATCCGCCTCAAGCCCACCCTTCATGGGCCAACACCTCTGCTATACAGACTGGG
CGCTGTTCAGAATGAAATCACCCTGACGCACCCGATCACCAAGTACATCATGACATGCATGTCGGCTGACCT
Donor 66719/ Panel Number 10024/ Transmitted Variant #1 (SEQ ID NO: 3)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGTAGACGTCAGCCTATCCCCAAGGCACGTCGGCCCGAGGGCAGGACCTGGGCTCAGCC
CGGGTACCCTTGGCCCCTCTACGGCAATGAGGGCTGCGGATGGGCGGGATGGCTCCTGTCTCCCCGTGGCTCTCGGCCTAGTTGGGGCCCCAC
AGACCCCCGGCGTAGGTCGCGCAATTTGGGTAAGGTCATCGATACCCTTACGTGCGGCTTCGCCGACCTCATGGGGTACATACCGCTCGTCGG
CGCCCCCCTTGGAGGCGCTGCCAGGGCCCTGGCGCACGGCGTCCGGGTTCTGGAAGACGGCGTGAACTATGCAACAGGGAATCTTCCTGGTTG
CTCTTTCTCTATCTTCCTCCTGGCCCTGCTCTCTTGCTTGACCGTGCCCGCTTCGGCCTACCAAGTGCGCAACTCCACGGGGCTTTATCATGT
CACCAACGACTGCCCTAATTCGAGTATTGTGTACGAGGCGGCCGATGCTATCCTGCACACTCCGGGGTGTGTCCCTTGCGTTCGCGAGGGTAA
CGTCTCGAGGTGCTGGGTGGCGATGACCCCCACGGTGGCCACTAGGGACGGCAAGCTCCCCACAACGCAGCTTCGACGCCACATCGATCTGCT
TGTCGGGAGCGCCACCCTTTGCTCGGCCCTCTACGTGGGGGACCTGTGCGGGTCTGTCTTTCTTGTCGGTCAGATGTTTACTTTCTCTCCCAG
GCGCCACTGGACGACGCAAGACTGCAATTGTTCCATGTACCCCGGCCATATAACGGGTCACCGCATGGCATGGGATATGATGATGAACTGGTC
CCCTACGACGGCGTTGATAGTAGCTCAGCTGCTCCGGATCCCACAAGCCATCTTGGACATGATCGCTGGTGCTCACTGGGGAGTCCTGGCGGG
CATTGCGTACTTTTCCATGGTGGGGAACTGGGCGAAGGTCCTGGTAGTGCTGCTGTTATTTGCCGGCGTCGACGCGGGAACCCACGTCAGCGG
GGGAGCCGTCGCCCGTGGCGCAGCTTCAATTGCCGGGTTGTTTAACTCAGGTGCCAAACAGAACATCCAGCTGATCAACACCAACGGCAGTTG
GCACATCAATAGCACGGCCTTGAACTGCAATGATAGCCTTAACACCGGCTGGATAGCAGGGCTTTTCTACTACAACAAATTCAACTCTTCAGG
CTGTCCCGACAGGTTGGCCAGCTGCCGGCGCCTTACCGATTTTGCCCAGGGCTGGGGCCCTATCAGTCACGCCAACGGAAGTGGCCCCGACGA
ACGCCCTTACTGCTGGCACTACCCCCCAAGACCTTGTGGTATTGTGCCGGCAAAGAGCGTGTGTGGCCCAGTATATTGCTTCACCCCCAGCCC
TGTGGTGGTGGGGACGACCGACAAGTCGGGCGCGCCTACCTACAACTGGGGTGATAATGATACGGACGTTTTCGTCCTTAACAACACTAGGCC
ACCGCTGGGCAATTGGTTCGGTTGTACCTGGATGAACTCAACTGGATTCACCAAAGTGTGCGGAGCGCCCCCCTGTGTCATCGGAGGGGTGGG
CAATAACACCTTGCGCTGCCCCACTGATTGTTTCCGCAAGCATCCGGAAGCCACGTACTCTCGGTGCGGCTCTGGTCCCTGGATTACACCCAG
GTGCCTGGTCGACTATCCGTATAGGCTGTGGCATTATCCTTGTACCATCAACTACACCATCTTCAAAGTCAGGATGTACGTGGGAGGGGTCGA
GCACAGGCTGGAAGCTGCCTGCAACTGGACGCGGGGCGAACGTTGTGATCTGGACGACAGGGACAGGTCCGAGCTCAGCCCGTTGCTGCTGTC
CACCACGCAGTGGCAGATCCTTCCGTGTTCCTTCACGACCCTGCCAGCCTTGTCCACCGGCCTCATCCACCTCCACCAGAACATTGTGGACGT
GCAGTACTTGTACGGGGTGGGGTCAAGCATCGCGTCCTGGGCCATTAAATGGGACTACGTCGTCCTCCTCTTCCTTTTGCTTGCAGACGCGCG
CGTCTGCTCCTGCTTGTGGATGATGTTACTCATATCCCAAGCGGAGGCGGCTTTGGAGAACCTCGTAGTACTCAATGCAGCATCCCTGGCCGG
GACACACGGTCTTGCATCCTTCCTCGTGTTCTTCTGCTTTGCGTGGTATCTGAAGGGTAGGTGGGTGCCCGGAGCAGTCTACGCCCTCTACGG
GATGTGGCCTCTCCTCCTGCTCCTGTTGGCGTTGCCCCAGCGGGCATACGCACTGGACACGGAGGTGGCCGCGTCGTGTGGCGGCGTTGTCCT
TGTCGGGGTAATGGCGCTGACTCTGTCACCATATTACAAGCACTATATCGGCTGGTGCTTGTGGTGGCTTCAGTATTTTCTGACTAGAGCAGA
AGCGCAACTGCATGTGTGGGTTCCCCCCCTCAACGTTCGAGGGGGGCGCGATGCCGTCATCTTACTCATGTGTGTTGTACACCCGACCTTGGT
ATTTGACATCACCAAGCTACTGCTGGCCGTCTTCGGACCCCTTTGGGTTCTGCAAGCCAGCCTGCTTAAGGTGCCCTACTTCGTGCGCGTTCA
AGGCCTTCTCCGGCTCTGCGCGCTAGCGCGGAAGGTGGTCGGAGGCCATTACGTGCAAATGGTCATCATCAAGTTGGGGGCGCTTACTGGCAC
TTATGTCTATAACCATCTCACTCCTCTTCGGGACTGGGCGCACAACGGCCTCCAAGATCTGGCCGTGGCTGTAGAGCCAGTCGTCTTCTCCCG
AATGGAAACCAAGCTCATCACGTGGGGGGCAGACACTGCCGCGTGCGGTGATATCATCAACGGCTTGCCCGTCTCCGCCCGCAGGGGGCAGGA
GATACTGCTCGGACCAGCCGATGGAATGGTCTCCAAGGGGTGGAGGTTGCTGGCGCCCATCACGGCGTACGCCCAGCAGACGAGGGGCCTCCT
AGGGTGCATAATCACCAGCCTGACTGGCCGAGACAAAAACCAAGTGGAGGGTGAAGTCCAGATTGTGTCAACTGCTGCCCAAACTTTTCTGGC
AACGTGCATCAATGGGGTATGCTGGACTGTCTACCATGGGGCTGGAACGAGGACCATTGCATCACCCAAGGGTCCTGTTATCCAGATGTATAC
CAATGTGGACAAAGACCTTGTGGGCTGGCCCGCCCCTCAAGGTTCCCGCTCATTGACACCCTGCACCTGCGGCTCCTCGGACCTCTACTTGGT
CACGAGGCATGCCGATGTCATTCCCGTGCGCCGGCGAGGTGATAGCAGGGGCAGCCTGCTTTCGCCCCGGCCCATTTCCTACTTGAAAGGCTC
CTCGGGGGGTCCGCTGTTGTGCCCCGCGGGGCACGCCGTGGGCATATTCAGGGCCGCGGTGTGCACCCGTGGAGTGGCTAAGGCGGTGGACTT
CATCCCTGTGGAGAACCTAGAGACAACCATGAGGTCCCCGGTGTTTACGGACAACTCCTCTCCACCAGCAGTGCCCCAGAGCTTCCAGGTGGC
CCACCTGCACGCTCCCACCGGCAGCGGCAAGAGCACCAAGGTCCCGGCTGCATACGCGGCTCAGGGCTATAAAGTGCTAGTGCTCAACCCCTC
CGTTGCTGCAACACTGGGCTTTGGTGCTTACATGTCCAAGGCCCATGGGGTCGAGCCTAATATCAGGACCGGGGTGAGAACAATTACCACTGG
CAGCCCCATTACGTACTCCACCTATGGCAAGTTCCTTGCCGACGGCGGGTGTTCAGGGGGCGCCTATGACATAATAATTTGTGATGAGTGCCA
CTCCACGGATGCCACATCCATCTTGGGCATCGGCACTGTCCTCGACCAAGCAGAGACTGCGGGGGCGAGACTGGTTGTGCTCGCCACCGCTAC
TCCTCCGGGCTCTGTCACTGTGCCCCATCCTAACATCGAGGAGGTTGCTCTGTCCACCACCGGAGAGGTCCCTTTTTACGGCAAGGCCATCCC
CCTTGAGGTAATCAAAGGGGGGAGACATCTCATCTTCTGTCACTCAAAGAAGAAGTGCGATGAGCTCGCCGCAAAGCTGGTCGCGTTGGGCGT
CAATGCCGTGGCCTACTACCGCGGTCTTGACGTTTCTGTCATCCCAACCAGCGGCGATGTTGTCGTCGTGGCGACCGACGCTCTCATGACCGG
CTTTACCGGCGACTTCGACTCGGTGATAGACTGCAACACGTGTGTCACTCAGACAGTCGATTTCAGCCTTGACCCTACCTTCACCATTGAGAC
AACCACGCTCCCCCAGGATGCTGTCTCCCGTACTCAACGTCGGGGCAGGACTGGCAGGGGGAAGCCAGGCATCTACAGATTTGTGGCACCGGG
AGAGCGCCCCTCTGGCATGTTCGACTCGTCTGTCCTCTGCGAGTGCTATGACGCGGGCTGTGCTTGGTATGAGCTGACGCCCGCCGAGACTAC
AGTTAGGCTACGAGCGTACATGAACACCCCGGGGCTTCCCGTGTGCCAGGACCATCTTGAGTTTTGGGAGGGCGTCTTTACGGGCCTCACCCA
TATAGATGCCCATTTTCTGTCCCAGACAAAGCAGAGTGGGGAGAACCTTCCTTACCTGGTAGCGTACCAAGCCACCGTGTGCGCTAGGGCTCA
AGCCCCTCCCCCATCGTGGGACCAGATGTGGAAGTGTTTGATCCGCCTCAAACCCACCCTCCACGGGCCAACACCCCTGCTATACAGACTGGG
TGCTGTTCAGAATGAAGTCACCCTGACGCACCCAATCACCAAATACATCATGA
Donor 66719/ Panel Number 10024/ Transmitted Variant #2 (SEQ ID NO: 4)
CCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGTGGTAGACGTCAGCCTATCCCCAAGGCACGTCGGCCCGAGGGCAGGACCTGGGCTCAGCC
CGGGTACCCTTGGCCCCTCTATGGCAATGAGGGCTGCGGGTGGGCGGGATGGCTCCTGTCTCCCCGTGGCTCTCGGCCTAGTTGGGGCCCCAC
AGACCCCCGGCGTAGGTCGCGCAATTTGGGTAAGGTCATCGATACCCTTACGTGCGGCTTCGCCGACCTCATGGGGTACATACCGCTCGTCGG
CGCCCCTCTTGGAGGCGCTGCCAGGGCCCTGGCGCACGGCGTCCGGGTTCTGGAAGACGGCGTGAACTATGCAACAGGGAACCTTCCTGGTTG
CTCTTTCTCTATCTTCCTCCTAGCCCTGCTCTCTTGCTTGACCGTGCCCGCTTCGGCCTACCAAGTGCGCAACTCCACGGGGCTTTATCATGT
CACCAACGACTGCCCTAATTCGAGTATTGTGTACGAGGCGGCCGATGCTATCCTGCACACTCCGGGGTGTGTCCCTTGCGTTCGCGAGGGTAA
CGTCTCGAGGTGCTGGGTGGCGATGACCCCCACGGTGGCCACTAGGGACGGCAAGCTCCCCACAACGCAGCTTCGACGCCACATCGATCTGCT
TGTCGGGAGCGCCACCCTTTGCTCGGCCCTCTACGTGGGGGACCTGTGCGGGTCTGTCTTTCTTGTCGGTCAGATGTTTACTTTCTCTCCCAG
GCGCCACTGGACAACGCAAGACTGCAATTGTTCCATGTACCCCGGCCATATAACGGGTCACCGCATGGCATGGGATATGATGATGAACTGGTC
CCCTACGACGGCATTGATAGTGGCTCAGCTGCTCCGGATCCCACAAGCCATCTTGGACATGATCGCTGGTGCTCACTGGGGAGTCCTGGCGGG
CATTGCGTATTTCTCCATGGTGGGGAACTGGGCGAAGGTCCTGGTAGTGCTGCTGTTATTTGCCGGCGTCGACGCGGGAACCCACGTCAGCGG
GGGATCCATCGCCCGTGGCGCAGCTTCAATTGCCGGGTTGTTTAACCAAGGTGCCAGACAGAACATCCAGCTGATCAACACCAACGGCAGTTG
GCACATCAATAGCACGGCCTTGAATTGCAATGATAGCCTTAACACCGGCTGGATAGCAGGGCTTTTCTATTACAACAAATTCAACTCTTCAGG
CTGTCCCGAGAGGTTGGCCAGCTGCCGGCGCCTTACCGATTTTGCCCAGGGCTGGGGCCCTATCAGTTATGCCAACGGAAGTGGCCCCGACGA
ACGCCCTTACTGCTGGCACTACCCTCCAAGACCTTGTGGTATTGTGCCGGCAAAGAGCGTGTGTGGCCCAGTATATTGCTTCACCCCCAGCCC
TGTGGTGGTGGGGACGACCGACAAGTCGGGCGCGCCTACCTACAACTGGGGTGATAATGATACGGACGTTTTCGTCCTTAACAACACTAGGCC
ACCGCTGGGCAATTGGTTCGGTTGTACCTGGATGAACTCAACTGGATTCACCAAAGTGTGCGGAGCGCCCCCCTGTGTCATCGGAGGGGTGGG
CAATAACACCTTGCGCTGCCCCACTGATTGTTTCCGCAAGCATCCGGAAGCCACGTACTCTCGGTGCGGCTCTGGTCCCTGGATTACACCCAG
GTGCCTGGTCGACTATCCGTATAGGCTGTGGCATTATCCTTGTACCATCAACTACACCATCTTCAAAGTCAGGATGTACGTGGGAGGGGTCGA
GCACAGGCTGGAAGCTGCCTGCAATTGGACGCGGGGCGAACGTTGTGATCTGGACGACAGGGACAGGTCCGAGCTCAGCCCGTTGCTGCTGTC
CACCACGCAGTGGCAGGTCCTTCCGTGTTCCTTCACGACCCTGCCAGCCTTGTCCACCGGCCTCATCCACCTCCACCAGAACATTGTGGACGT
GCAGTACTTGTACGGGGTGGGGTCAAGCATCGCGTCCTGGGCCATTAAGTGGGACTACGTCGTCCTCCTGTTCCTTTTGCTTGCAGACGCGCG
CGTCTGCTCCTGCTTGTGGATGATGTTACTCATATCCCAAGCGGAGGCGGCTTTGGAGAACCTCGTAGTACTCAATGCAGCATCCCTGGCCGG
GACACACGGTCTTGCATCCTTCCTCGTGTTCTTCTGCTTTGCATGGTATCTGAAGGGTAGGTGGGTGCCCGGAGCAGTCTACGCCCTCTACGG
GATGTGGCCTCTCCTCCTGCTCCTGTTGGCGTTGCCCCAGCGGGCATACGCACTGGACACGGAGGTGGCCGCGTCGTGTGGCGGCGTTGTCCT
TGTCGGGGTAATGGCGCTGACTCTGTCACCATATTATAAGCACTATATCAGCTGGTGCTTGTGGTGGCTTCAGTATTTTCTGACTAGAGCAGA
AGCGCAACTGCATGTGTGGGTTCCCCCCCTCAACGTTCGAGGGGGGCGCGACGCCGTCATCTTACTCATGTGTGCTGTACACCCGACCTTGGT
ATTTGACATCACCAAGCTACTGCTGGCCGTCCTCGGACCCCTTTGGGTTCTGCAAGCCAGTCTGCTTAAGGTGCCCTACTTCGTGCGCGTTCA
AGGCCTTCTCCGGCTCTGCGCGTTAGCGCGGAAGGTGGTCGGAGGCCATTACGTGCAAATGGTCATCATCAAGTTGGGGGCGCTTACTGGCAC
TTATGTCTATAACCATCTCACTCCTCTTCGGGACTGGGCGCACAACGGCCTTCAAGATCTGGCCGTGGCTGTAGAGCCAGTCGTCTTCTCCCG
AATGGAAACCAAGCTCATCACGTGGGGGGCAGACACTGCCGCGTGCGGTGATATCATCAACGGCTTGCCCGTCTCCGCCCGCAGGGGGCAGGA
GATACTGCTCGGACCAGCCGACGGAATGGTCTCCAAGGGGTGGAGGTTGCTGGCGCCCATCACGGCGTACGCCCAGCAGACGAGGGGCCTCCT
AGGGTGCATAATCACCAGCCTGACTGGCCGAGACAAAAACCAAGTGGAGGGTGAAGTCCAGATTGTGTCAACTGCTGCCCAAACTTTCCTGGC
AACGTGCATCAATGGGGTATGCTGGACTGTCTACCATGGGGCTGGAACGAGGACCATTGCATCACCCAAGGGTCCTGTTATCCAGATGTATAC
CAATGTGGACAAGGACCTCGTGGGCTGGCCCGCCCCTCAGGGTTCCCGCTCATTGACACCCTGCACCTGCGGCTCCTCGGACCTTTACTTGGT
CACGAGGCATGCCGATGTCATTCCCGTGCGCCGGCGAGGTGATAGCAGGGGCAGCCTGCTTTCGCCCCGGCCCATTTCCTACTTGAAAGGCTC
CTCGGGGGGTCCGCTGTTGTGCCCCGCGGGACACGCCGTAGGCATATTCAGGGCCGCGGTGTGCACCCGTGGAGTGGCTAAGGCGGTGGACTT
TATCCCTGTAGAGAACCTAGAGACAACCATGAGGTCCCCGGTGTTTACGGACAACTCCTCTCCACCAGCAGTGCCCCAGAGCTTCCAGGTGGC
CCACCTGCACGCTCCCACCGGCAGCGGCAAGAGCACCAAGGTCCCGGCTGCATACGCGGCTCAGGGCTATAAAGTGCTAGTGCTCAACCCCTC
CGTTGCTGCAACACTGGGCTTTGGTGCTTACATGTCCAAGGCCCATGGGGTCGAGCCTAATATCAGGACCGGGGTGAGAACAATTACCACTGG
CAGCCCCATTACGTACTCCACCTACGGCAAGTTCCTTGCCGACGGCGGGTGTTCAGGGGGTGCTTATGACATAATAATTTGTGATGAGTGCCA
CTCCACGGATGCCACATCCATCTTGGGCATCGGCACTGTCCTCGACCAAGCAGAGACTGCGGGGGCGAGACTGGTTGTGCTCGCCACCGCTAC
TCCTCCGGGCTCTGTCACTGTGCCCCATCCTAACATCGAGGAGGTTGCTCTGTCCACCACCGGAGAGGTCCCTTTTTACGGCAAGGCCATCCC
CCTTGAAGTAATCAAAGGGGGGAGACACCTCATCTTCTGTCACTCAAAGAAGAAGTGCGACGAGCTCGCCGCAAAGCTGGTTGCGTTGGGCGT
CAATGCCGTGGCCTACTACCGCGGTCTTGACGTTTCTGTCATCCCGACCAGCGGCGATGTTGTCGTCGTGGCAACCGACGCTCTCATGACCGG
CTTTACCGGCGACTTCGACTCGGTGATAGACTGTAACACGTGTGTCACCCAGACAGTCGATTTCAGCCTTGACCCTACCTTCACCATTGAGAC
AACCACGCTCCCCCAGGATGCTGTCTCCCGTACTCAACGTCGGGGCAGGACTGGCAGGGGGAAGCCAGGCATCTACAGATTTGTGGCACCGGG
AGAGCGCCCCTCCGGCATGTTCGACTCGTCCGTCCTCTGTGAGTGCTATGACGCGGGCTGTGCTTGGTATGAGCTGACGCCCGCCGAGACTAC
AGTTAGGCTACGAGCGTACATGAACACCCCGGGGCTTCCCGTGTGCCAGGACCATCTTGAGTTTTGGGAGGGCGTCTTTACGGGCCTCACCCA
TATAGATGCCCATTTTCTGTCCCAGACAAAGCAGAGTGGGGAGAACCTTCCTTACCTGGTAGCGTACCAAGCCACCGTGTGCGCTAGGGCTCA
AGCCCCTCCCCCATCGTGGGACCAGATGTGGAAGTGTTTAATCCGCCTCAAACCCACCCTCCACGGGCCAACACCCCTGCTATACAGACTGGG
TGCTGTTCAGAATGAAATCACCCTGACACACCCAATCACCAAATACATCATGA
Donor 66747/ Panel Number 10012/ Transmitted Variant #1 (SEQ ID NO: 5)
TCGCCCACAGGACGTTAAGTTCCCGGGTGGCGGTCAGATCGTTGGTGGAGTTTACTTGTTGCCGCGCAGGGGCCCTAGAATGGGTGTGCGCGC
GCCGAGGAAGACTTCCGAGCGGTCGCAACCTCGAGGTAGACGTCAGCCTATCCCCAAGGCACGTCGGCCCGAGGGCAGGACCTGGGCTCAGCC
CGGGTACCCTTGGCCCCTCTATGGCAATGAGGGCTGCGGGTGGGCGGGATGGCTCCTGTCCCCCCGCGGCTCTCGGCCTAGTTGGGGCCCCAC
AGACCCCCGGCGTAGGTCGCGCAATTTGGGTAAGGTCATCGACACCCTTACGTGCGGCTTCGCCGACCTCATGGGGTACATACCGCTCGTCGG
CGCCCCTCTTGGAGGCGCTGCCAGGGCCCTGGCGCATGGCGTTCGGGTTCTGGAAGACGGCGTGAACTATGCAACAGGGAACCTTCCTGGTTG
CTCTTTCTCTATCTTCTTTCTGGCCCTGCTCTCTTGTCTGACTGTGCCCGCTTCGGCCTACCAAGTGCGCAACTCTACAGGGCTCTACCACGT
CACCAATGATTGCCCTAACTCGAGTATTGTGTACGAGGCGCCTGATGCCATCCTGCACACTCCGGGGTGTGTCCCTTGCGTTCGCGAGGGTAA
CGCCTCGAGGTGTTGGGTGGCGATGACCCCCACGGTGGCCACCAGGGACGGCAAACTCCCCGCGACGCAGCTTCGACGTCACATCGATCTGCT
TGTCGGGAGCGCCACCCTCTGTTCGGCTCTTTACGTGGGGGACCTGTGCGGGTCTGTCTTTCTTGTGGGTCAACTGTTTACCTTCTCCCCCAG
GCGCCACTGGACGACGCAAGACTGCAATTGCTCTATCTATCCCGGCCATATAACGGGTCACCGCATGGCATGGGATATGATGATGAACTGGTC
CCCGACGACGGCGTTGGTAATGGCTCAGCTGCTCCGGATCCCGCAAGCCATCTTGGACATGATCGCTGGTGCCCACTGGGGAGTCTTGGCGGG
CATAGCGTATTTCTCCATGGTGGGGAACTGGGCGAAGGTCCTGGTAGTGCTGCTGCTATTCGCCGGCGTCGACGCGGAAACCATGACCACCGG
GGGGGCCGCCGCTCGCAACGTGTATAGAATTTCCAGCTTCTTCTCGCCGGGCCCCCAGCAGAACATCCAGCTGATCAACACCAACGGCAGTTG
GCACATCAATAGGACGGCCTTGAACTGCAACGACAGCCTTAACACCGGCTGGTTAGCAGGGCTTTTCTATCATCGCAGCTTCAACTCTTCAGG
CTGTCCTGCGAGGCTGGCCAGCTGCCGACGCCTTACCGACTTTGCCCAGGGCTGGGGCCCTATCAGCCATGCCAACGGCAGCGGCCCCGACCA
ACGCCCCTACTGTTGGCACTACCCCCCAAAACCTTGCGGTATTGTGCCCGCACGGAACGTGTGTGGCCCGGTATATTGCTTCACCCCCAGTCC
CGTGGCGGTGGGAACGACCGACAGGGCGGGCGTCCCTACCTACCGCTGGGGTGAAAATGAAACGGACGTCTTCGTCCTTAACAACACCAGGCC
ACCGCTGGGCAATTGGTTCGGTTGTACCTGGATGAATTCAACTGGATTCACCAAAGTGTGCGGAGCGCCTCCTTGTGTCATCGGAGGAGTGGG
CAACGACACCTTGTACTGCCCCACTGATTGTTTCCGCAAGCATCCGGAAGCCACATATTCCCGGTGCGGCTCCGGTCCCTGGATTACACCCAG
ATGCCTGGTTGACTACCCGTATAGGCTTTGGCATTATCCTTGTACCGTCAACTACACCATATTCAAAGTCAGGATGTATGTGGGAGGGGTCGA
GCATAGGCTGGATGCTGCCTGCAACTGGACGCGGGGCGAACGTTGCGATCTGGAAGACAGGGACAGGTCCGAGCTCAGCCCGTTGCTGCTGTC
CACTACACAGTGGCAGGTCCTTCCGTGCTCCTTCACGACCTTGCCAGCCTTGTCCACCGGCCTCATCCACCTCCACCAGAACATTGTGGACGT
GCAGTACCTGTACGGGGTAGGGTCAAGCATCGCGTCCTGGGCCATTAAGTGGGAGTACGTCGTTCTCCTGTTCCTTCTGCTTGCAGACGCGCG
CGTCTGCTCCTGCCTGTGGATGATGCTACTCATATCCCAAGCGGAGGCGGCTTTGGAGAACCTCGTAATACTCAATGCAGCATCCCTGGCCGG
GACGCAAGGTCTTGTATCCTTCCTCGTGTTCTTCTGCTTTGCGTGGTATCTGAAGGGTAGGTGGGTGCCAGGAGCGGTTTACGCCCTCTACGG
AATGTGGCCTCTCCTCCTGCTCCTGTTGGCGTTGCCCCAGCGGGCATACGCGCTGGACACGGAGGTGGCCGCGTCGTGTGGCGGCGTTGTTCT
TGTCGGGTTGATGATGCTGACTCTGTCACCGTATTACAAGCGCTATATCAGCTGGTGCTTATGGTGGCTTCAGTATTTTCTGACTAGGGCAGA
AGCGCAACTGCACGTGTGGGTTCCCCCCCTCAACGTTCGAGGGGGGCGCGACGCTGCCATCTTACTCACATGCGTTGTACACCCGACTCTGGT
ATTTGACATCACCAAACTACTGCTGGCTGTCCTCGGACCCCTTTGGATTCTTCAAGCCAGTTTGCTTAAAGTACCCTACTTCGTGCGCGTCCA
AGGCCTTCTCCGGATCTGCGCGTTAGCGCGGAAGATGGTCGGAGGCCACTACGTGCAAATGGTTATCATCAAGTTAGGGATGCTTACTGGCAC
CTATGTTTATAACCATCTCACCCCCCTCCGGGACTGGGCGCACAACGGCCTGCGAGATCTGGCCGTGGCTGTAGAACCAGTCGTCTTCTCCCA
AATGGAGACCAAGCTCATCACGTGGGGGGCAGACACTGCCGCGTGCGGTGACATCATCAACGGCTTGCCCGTCTCCGCCCGTAGGGGCCAGGA
GATACTGCTCGGACCAGCCGATGGAATGGTCTCCAAGGGGTGGAGGTTGCTGGCGCCCATCACGGCGTACGCCCAGCAGACAAGGGGCCTCCT
AGGGTGCATAATCACCAGCCTAACCGGCCGGGATAAAAACCAAGTGGAGGGTGAGGTCCAGATTGTGTCAACTGCTGCCCAAACTTTCCTGGC
AACGTGCATCAGTGGGGTATGCTGGACTGTCTACCACGGGGCCGGAACGAGGACTATTGCATCATCCAAAGGCCCTGTTATCCAGATGTATAC
CAATGTGGACAAAGACCTTGTGGGCTGGCCCGCTCCTCAAGGTGCCCGCTCATTGACACCCTGCACCTGCGGCTCCTCGGACCTTTACCTGGT
CACGAGGCATGCCGATGTCATCCCCGTGCGCCGGCGGGGTGATAGCAGGGGCAGCCTGCTCTCGCCCCGGCCCATTTCCTATCTGAAAGGCTC
CTCGGGGGGCCCGCTGTTGTGCCCCGCGGGACACGCCGTAGGCATATTCAGGGCCGCGGTGTGCACCCGTGGAGTGGCTAAGGCGGTGGACTT
TATCCCTGTGGAGAACCTAGAGACAACCATGAGGTCCCCGGTGTTCACGGACAACTCCTCTCCGCCAGCAGTGCCCCAGAGCTTCCAGGTGGC
CCACCTGCATGCTCCCACAGGCAGCGGCAAGAGCACCAAGGTCCCGGCTGCGTATGCGGCTCAGGGCTATAAGGTGCTAGTACTCAACCCCTC
TGTCGCTGCAACACTGGGCTTTGGTGCTTATATGTCCAAGGCCCATGGGATCGATCCTAATATCAGGACCGGGGCGAGAACAATTACCACTGG
CAGCCCCATCACGTACTCCACCTACGGCAAATTTCTTGCCGACGGCGGGTGCTCGGGGGGCGCTTATGATATAATAATTTGTGACGAGTGCCA
TTCCACGGATGCCACATCCATCTTGGGCATCGGCACTGTCCTTGACCAGGCAGAGACCGCGGGGGCGAGACTGGTTGTGCTCGCCACCGCCAC
CCCTCCGGGCTCCGTCACTGTGCCCCATCCTAACATCGAGGAGGTTGCTCTGTCCACCACCGGAGAGATTCCTTTCTATGGCAAGGCTATCCC
CCTCGAAGTAATCAAAGGGGGGAGACATCTCATTTTTTGTCACTCAAAGAAGAAGTGCGACGAGCTCGCCGCAAAGCTAGTCGCATTGGGCAT
CAATGCCGTGGCCTACTACCGCGGTCTTGACGTGTCCGTCATCCCGACCAGCGGCGATGTTGTCGTCGTGGCAACTGATGCTCTCATGACCGG
CTTTACCGGCGACTTCGACTCGGTGATAGACTGCAACACGTGTGTCACCCAGACGGTCGACTTCAGCCTTGACCCTACTTTCACCATTGAGAC
AACCACGCTTCCCCAGGATGCTGTCTCCCGCACTCAACGTCGGGGTAGGACTGGCAGGGGGAAGCCAGGCATCTACAGATTTGTGGCACCGGG
GGAGCGCCCCTCCGGCATGTTCGACTCGTCCATCCTCTGTGAGTGCTATGACGCGGGCTGTGCTTGGTATGAGCTCACGCCCGCCGAGACTAC
AGTTAGACTACGAGCGTACATGAACACCCCGGGGCTTCCCGTGTGCCAAGACCATCTTGAATTTTGGGAGGGCGTCTTCACAGGCCTCACTCA
TATAGATGCCCACTTCCTATCCCAAACAAAGCAGAGTGGGGAGAATCTTCCTTACCTGGTAGCGTACCAAGCCACCGTGTGCGCTAGGGCTCA
AGCCCCTCCCCCATCGTGGGACCAGATGTGGAAGTGTTTGATCCGCCTCAAACCCACCCTCCATGGGCCAACACCTCTGCTATACAGACTGGG
CGCTGTCCAGAATGAGGTCACCCTGACGCACCCAGTCACCAAATACATTATGA
Donor 66747/ Panel Number 10012/ Transmitted Variant #2 (SEQ ID NO: 6)
TCGCCCACAGGACGTTAAGTTCCCGGGTGGCGGTCAGATCGTTGGTGGAGTTTACTTGTTGCCGCGCAGGGGCCCTAGAATGGGTGTGCGCGC
GCCGAGGAAGACTTCCGAGCGGTCGCAACCTCGAGGTAGACGTCAGCCTATCCCCAAGGCACGTCGGCCCGAGGGTAGGACCTGGGCTCAGCC
CGGGTACCCTTGGCCCCTCTATGGCAATGAGGGCTGCGGGTGGGCGGGATGGCTCCTGTCTCCCCGCGGCTCTCGGCCTAGTTGGGGCCCCAC
AGACCCCCGGCGTAGGTCGCGCAATTTGGGTAAGGTCATCGATACCCTTACGTGCGGCTTCGCCGACCTCATGGGGTACATACCGCTCGTCGG
CGCCCCTCTTGGAGGCGCTGCCAGGGCCCTGGCGCATGGCGTCCGGGTTCTGGAAGACGGCGTGAACTATGCAACGGGGAACCTTCCCGGTTG
CTCTTTCTCTATCTTCCTTCTGGCCCTGCTCTCTTGTCTGACTGTGCCCGCTTCGGCCTACCAAGTGCGCAACTCCACGGGGCTTTACCACGT
CACCAATGATTGCCCCAACTCGAGTATTGTGTACGAGGCGCCTGATGCCATCCTGCACACTCCGGGGTGTGTCCCTTGCGTTCGCGAGGGTAA
CGCCTCGAGGTGTTGGGTGGCGATGACCCCCACGGTGGCCACCAGGGACGGCAAACTCCCTGCAACGCAGCTCCGACGTCACATCGATCTGCT
TGTCGGGAGTGCCACCCTCTGTTCGGCCCTCTACGTGGGGGACCTGTGCGGGTCTGTCTTTCTTGTTGGTCAACTGTTTACCTTCTCTCCCAG
GCGCCACTGGACGACGCAAGACTGCAATTGTTCTATCTATCCCGGCCATATAACGGGCCACCGCATGGCATGGGATATGATGATGAACTGGTC
CCCGACGACGGCGTTGGTAATGGCTCAGCTGCTCCGGATCCCGCAAGCCATCTTGGATATGATCGCTGGTGCCCACTGGGGAGTCCTGGCGGG
CATAGCGTATTTCTCCATGGTGGGGAACTGGGCGAAGGTCCTGGTAGTGCTGCTGCTATTTGCCGGCGTCGATGCGGGAACCGTCGTCACCGG
AGGGGTCGCCGCCCGCACCGCGTATGGATTTACTAGTCTCTTCACGCGAGGCGCCCAGCAGAACATCCAGCTGATCAACACCAACGGCAGTTG
GCACATCAATAGGACGGCCTTGAACTGCAACGACAGCCTTAACACCGGCTGGTTAGCAGGGCTTTTCTATCGGAACAACTTCAACTCTTCAGG
CTGTCCTCAGAGGTTGGCCAGCTGCCGGCGCCTTGCCGATTTTGCCCAGGGCTGGGGCCCTATCAGCCATGCCAACGGCAGCGGCCCCGACCA
CCGCCCCTACTGCTGGCACTACCCCCCAAAACCTTGCGGCATTGTGCCCGCACAGAACGTGTGTGGCCCGGTATATTGCTTCACTCCCAGCCC
CGTGGCGGTGGGAACGACCGACAGGGCGGGCATCCCTACCTACAGCTGGGGTGAAAATGATACGGACGTCTTCGTCCTTAACAACACCAGGCC
ACCGCTGGGCAATTGGTTTGGTTGCACCTGGATGAACTCAACTGGATTCACCAAAGTGTGTGGAGCGCCCCCTTGTGTCATCGGAGGGGTGGG
CAACAACACCTTGCACTGCCCCACTGATTGTTTCCGCAAGCATCCGGAAGCCACATACTCCCGGTGCGGCTCCGGTCCCTGGATTACACCCAG
ATGCCTGGTCGACTACCCGTATAGGCTTTGGCATTATCCTTGTACCATCAACTATACCATATTCAAAGTCAGGATGTATGTGGGAGGGGTCGA
GCATAGGCTGGATGCTGCCTGCAACTGGACGCGGGGCGAACGTTGCGATCTGGAAGACAGGGACAGGTCCGAGCTCAGCCCGTTGCTGCTGTC
CACTACACAGTGGCAGGTCCTTCCGTGTTCCTTCACGACCTTGCCAGCCTTGTCCACCGGTCTCATCCACCTCCACCAGAACATCGTGGACGT
GCAGTACCTGTACGGGGTGGGGTCAAGCATCGCGTCCTGGGCCATCAAGTGGGAGTACGTCGTTCTCCTGTTCCTTCTGCTTGCAGACGCGCG
CGTCTGCTCCTGCCTGTGGATGATGCTACTCATATCCCAAGCGGAGGCGGCTTTGGAGAACCTCGTAATACTCAATGCAGCATCCCTGGCCGG
GACGCAAGGTCTTGTATCCTTCCTCGTGTTCTTCTGCTTTGCATGGTATCTGAAGGGTAGGTGGGTGCCAGGAGCGGTTTACGCCCTCTACGG
AATGTGGCCTCTCCTCCTGCTCCTGCTGGCGTTGCCCCAGCGGGCATACGCGCTGGACACGGAGGTGGCCGCGTCGTGTGGCGGCGTTGTTCT
CGTCGGGTTGATGGCGCTGACTCTGTCACCGTATTACAAGCGCTACATCAGCTGGTGCTTATGGTGGCTCCAGTATTTTCTGACTAGAGCAGA
AGCGCAACTGCACGTGTGGGTTCCCCCCCTCAACGTTCGAGGGGGGCGCGACGCCGTCATCTTACTCATGTGTGTTGTACACCCGACTTTGGT
ATTTGACATCACCAAACTACTGCTGGCCGTCCTCGGACCCCTTTGGATTCTTCAAGCCAGTTTGCTTAAAGTACCCTACTTCGTGCGCGTCCA
AGGCCTTCTCCGGATCTGCGCGTTAGCGCGGAAGATGGTCGGGGGCCAATACGTGCAAATGGTTATCATCAAGTTAGGGACGCTTACTGGCAC
CTATGTTTACAACCATCTCACTCCTCTTCGGGACTGGGCGCACAACGGCCTGCGAGATCTGGCCGTGGCTGTAGAGCCAGTCGTCTTCTCCCA
AATGGAGACCAAGCTCATCACGTGGGGGGCAGACACTGCCGCGTGCGGTGACATCATCAACGGTCTGCCCGTCTCCGCCCGTAGGGGTCGGGA
GATACTGCTCGGACCAGCCGATGGAATGGTCTCCAAGGGGTGGAGGTTGCTGGCGCCCATCACGGCGTACGCCCAGCAGACAAGGGGCCTCCT
AGGGTGCATAATCACCAGCCTAACCGGCCGGGACAAAAACCAAGTGGAGGGTGAGGTCCAGATTGTGTCAACTGCTGCCCAAACTTTCCTGGC
AACGTGCATCAGTGGGGTATGCTGGACTGTCTACCACGGGGCCGGAACGAGGACTATTGCATCATCCAAAGGCCCCGTTATCCAGATGTATAC
CAATGTGGACAAAGACCTTGTGGGCTGGCCCGCTCCTCAAGGTGCCCGCTCATTGACACCCTGCACCTGCGGCTCCTCGGACCTTTACCTGGT
CACGAGGCATGCCGATGTCATCCCCGTGCGCCGGCGGGGTGATAGCAGGGGCAGCCTGCTCTCGCCCCGGCCCATTTCCTACTTGAAAGGCTC
CTCGGGGGGCCCGCTGTTGTGCCCCGCGGGACACGCCGTAGGCATATTCAGGGCCGCGGTGTGCACCCGTGGAGTGGCTAAGGCGGTGGACTT
TATCCCTGTGGAGAACCTAGAGACAACCATGAGGTCCCCGGTGTTCACGGACAACTCCTCTCCGCCAGCAGTGCCCCAGAGCTTCCAGGTGGC
CCACCTGCATGCTCCCACAGGCAGCGGCAAGAGCACCAAGGTCCCGGCTGCATACGCGGCTCAGGGCTATAAGGTGCTAGTGCTCAACCCTTC
TGTTGCTGCAACATTGGGCTTTGGTGCTTACATGTCCAAGGCCCACGGGATCGATCCTAATATCAGGACCGGGGCGAGAACAATTACTACTGG
CAGCCCCATCACGTACTCCACCTACGGTAAGTTCCTTGCCGACGGCGGGTGCTCGGGGGGCGCTTATGATATAATAATTTGTGACGAGTGCCA
CTCCACGGATGCCACATCCATCTTGGGCATCGGCACTGTCCTTGACCAGGCAGAGACCGCGGGGGCGAGACTGGTTGTGCTCGCCACCGCCAC
CCCTCCGGGCTCCGTCACTGTGCCCCACCCCAACATTGAGGAGGTTGCTCTGTCCACCACCGGAGAGATTCCTTTCTATGGCAAGGCTATCCC
CCTCGAAGTAATCAAAGGGGGGAGACATCTCATTTTTTGTCACTCAAAGAAGAAGTGCGACGAGCTCGCCGCAAAGCTGGTCGCATTGGGCAT
CAATGCCGTGGCCTACTACCGCGGTCTTGATGTGTCTGTCATCCCGACCAGCGGCGATGTTGTCGTCGTGGCAACTGATGCTCTCATGACCGG
CTTTACCGGCGACTTCGACTCGGTGATAGACTGCAACACGTGTGTCACCCAGACGGTCGATTTCAGCCTTGACCCTACCTTCACCATTGAGAC
AACCACGCTTCCCCAAGACGCTGTCTCCCGCACTCAACGTCGGGGCAGGACTGGCAGGGGGAAGCCAGGCATCTACAGATTTGTGGCACCGGG
GGAGCGCCCTTCCGGCATGTTCGACTCGTCCGTCCTCTGTGAGTGCTATGACGCGGGCTGTGCTTGGTATGAGCTCACGCCCGCCGAGACTAC
AGTTAGACTACGAGCGTACATGAACACCCCGGGGCTTCCCGTGTGCCAAGACCATCTCGAATTTTGGGAGGGCGTCTTCACAGGCCTCACTCA
TATAGATGCCCACTTCCTATCCCAGACAAAGCAGAGTGGGGAGAATCTTCCTTACCTGGTAGCGTACCAAGCCACCGTGTGCGCTAGGGCTCA
AGCCCCTCCCCCATCGTGGGACCAGATGTGGAAGTGTTTGATCCGCCTCAAACCCACCCTCCATGGGCCAACACCTCTGCTATACAGACTGGG
CGCTGTCCAGAATGAGGTCACCTTGACGCACCCAGTCACTAAATACATCATGA
Donor 66747/ Panel Number 10012/ Transmitted Variant #3 (SEQ ID NO: 7)
TCGCCCACAGGACGTTAAGTTCCCGGGTGGCGGTCAGATCGTTGGTGGAGTTTACTTGTTGCCGCGCAGGGGCCCTAGAATGGGTGTGCGCGC
GCCGAGGAAGACTTCCGAGCGGTCGCAACCTCGAGGTAGACGTCAGCCTATCCCCAAGGCACGTCGGCCCGAGGGCAGGACCTGGGCTCAGCC
CGGGTACCCTTGGCCCCTCTATGGCAATGAGGGCTGCGGGTGGGCGGGATGGCTCCTGTCTCCCCGCGGCTCTCGGCCTAGTTGGGGCCCCAC
AGACCCCCGGCGTAGGTCGCGCAATTTGGGTAAGGTCATCGATACCCTTACGTGCGGCTTCGCCGACCTCATGGGGTACATACCGCTCGTCGG
CGCCCCTCTTGGAGGCGCTGCCAGGGCCCTGGCGCATGGCGTCCGGGTCCTGGAAGACGGCGTGAACTATGCAACAGGGAACCTTCCTGGTTG
CTCTTTCTCTATCTTCCTTCTGGCCCTGCTCTCTTGTCTGACTGTGCCCGCTTCGGCCTACCAAGTGCGCAACTCCACGGGGCTCTATCACGT
CACCAATGATTGCCCTAACTCGAGTATTGTGTACGAGGCGCCTGATGCCATCCTGCACACTCCGGGGTGTGTCCCTTGCGTTCGCGAGGGTAA
TGCCTCGAGGTGTTGGGTGGCGATGACCCCCACGGTGGCCACCAGGGACGGCAAACTCCCTGCGACGCAGCTCCGACGTCACATCGATCTGCT
TGTCGGGAGCGCCACCCTCTGTTCGGCCCTCTACGTGGGGGACCTGTGCGGGTCTGTCTTTCTTGTTGGTCAACTGTTTACCTTCTCTCCCAG
GCGCCACTGGACGACGCAAGACTGCAATTGTTCTATCTATCCCGGCCATATAACGGGCCACCGCATGGCATGGGATATGATGATGAACTGGTC
CCCGACGACAGCGTTGGTAATGGCTCAGCTGCTCCGGATCCCGCAAGCCATCTTGGACATGATCGCTGGTGCTCACTGGGGAGTCCTGGCGGG
CATAGCGTATTTCTCCATGGTGGGGAACTGGGCGAAGGTCCTGGTAGTGCTGTTGCTATTCGCCGGCGTCGATGCGGAAACCGTCGTCACCGG
GGGGACCGCCGCCCGCAACGCGTACGGACTCTCTAGTCTCTTCATGCGGGGCCCCAAGCAGAACATCCAGCTGATCAACACCAACGGCAGTTG
GCACATCAATAGGACGGCCTTGAACTGCAACGATAGCCTTAACACCGGCTGGCTAGCAGGGCTTTTCTATCAGCACAACTTCAACTCTTCAGG
CTGTCCTGAGAGGCTGGCCAGCTGCCGACGCCTTGCCGATTTTGCCCAGGGCTGGGGCCCTATCAGCCATGCCAACGGCAGCGGCCCCGACCA
CCGCCCCTACTGCTGGCACTACCCCCCAAAACCTTGCGGTATTGTGCCCGCACAGAACGTGTGTGGTCCGGTATATTGCTTCACTCCCAGCCC
CGTGGCGGTGGGAACGACCGACAGGTCGGGCGTCCCTACCTACCGCTGGGGTGAAAATGAGACGGACGTCTTCGTCCTTAACAACACCAGGCC
ACCGCTGGGCAATTGGTTTGGTTGTACCTGGATGAACTCAACTGGATTCACCAAAGTGTGCGGAGCGCCTCCTTGTGTCATCGGAGGGGCGGG
CAACAACACCTTGCACTGCCCCACCGATTGTTTCCGCAAGCATCCGGAAGCCACATACTCCCGGTGCGGCTCCGGTCCCTGGATTACACCCAG
ATGCATGGTCGACTACCCGTATAGGCTTTGGCATTATCCTTGTACCATCAACTATACCATATTTAAAGTCAGGATGTACGTGGGAGGGGTCGA
GCATAGGCTGGATGTTGCCTGCAACTGGACGCGGGGCGAACGCTGCGATCTGGAAGACAGGGACAGGTCCGAGCTCAGCCCGTTGCTGCTGTC
CACCACACAGTGGCAGGTCCTTCCGTGTTCCTTCACGACCTTGCCGGCCTTGTCCACCGGCCTCATCCACCTCCACCAGAACATCGTGGACGT
ACAGTACCTGTACGGGGTGGGGTCAAGCATTGCGTCCTGGGCCATTAAGTGGGAGTACGTCGTTCTCCTGTTCCTTCTGCTTGCAGACGCGCG
CGTCTGCTCCTGCCTGTGGATGATGCTACTCATATCCCAAGCGGAGGCGGCTTTGGAGAACCTCGTAACACTCAATGCAGCATCCCTGGCCGG
GACGCAAGGTCTTGTATCCTTCCTCGTGTTCTTCTGTTTCGCGTGGTATCTGAAGGGTAGGTGGGTGCCAGGAGCGGTTTACGCCCTCTACGG
GATGTGGCCTCTCCTCCTGCTCCTGTTGGCGTTGCCCCAGCGGGCATACGCGCTGGACACGGAGGTGGCCGCGTCGTGTGGCGGCGTTGTTCT
CGTCGGGTTGATGGCGCTGACTCTGTCACCGTATTACAAGCGCTACATCAGCTGGTGCTTATGGTGGCTCCAGTATTTTCTGACTAGAGCAGA
AGCGCAACTGCACGTATGGGTTCCCCCCCTCAACGCTCGAGGGGGGCGCGACGCTGTCATCTTACTCATGTGTGTTGTACACCCGACTTTGGT
ATTTGACATCACCAAACTACTGCTGGCCGTCCTCGGACCCCTTTGGATTCTTCAAGCCAGTCTGCTTAAAGTACCCTACTTTGTGCGCGTCCA
GGGCCTTCTCCGGATCTGCGCGTTAGCGCGGAAGATGGTTGGGGGCCACTACGTGCAAATGGTTATCATCAAGCTAGGGACGCTTACTGGCAC
CTATGTTTACAACCATCTCACTCCTCTTCGGGACTGGGCGCACAACGGCCTGCGAGATCTGGCCGTGGCTGTAGAGCCAGTCGTCTTCTCCCA
AATGGAGACCAAGCTCATCACATGGGGGGCAGACACTGCCGCGTGCGGTGACATCATCAACGGCTTGCCCGTCTCCGCCCGTAGGGGCCGGGA
GATACTGCTCGGACCAGCCGATGGAATGGTCTCCAAGGGGTGGAGGTTGCTGGCGCCCATCACGGCGTACGCCCAGCAGACAAGGGGCCTCTT
AGGATGCATAATCACCAGCCTGACCGGCCGGGACAAAAACCAAGTGGAGGGTGAAGTCCAGATTGTGTCAACTGCTGCCCAAACTTTCCTCGC
AACGTGCATCAGTGGGGTATGCTGGACTGTCTACCACGGGGCCGGAACGAGGACTATTGCATCATCCAAAGGCCCTGTTATCCAGATGTATAC
CAATGTGGACAAAGACCTTGTGGGCTGGCCCGCCCCTCAAGGTGCCCGCTCATTGACGCCCTGCACCTGCGGCTCCTCGGACCTTTACCTGGT
CACGAGGCATGCCGATGTCATCCCCGTGCGCCGGCGGGGTGATAGCAGGGGCAGCCTGCTTTCGCCCCGGCCCATTTCCTATTTGAAAGGTTC
CTCGGGGGGCCCGCTGTTGTGCCCCGCGGGACACGCCGTAGGCATATTCAGGGCCGCGGTGTGCACCCGTGGAGTGGCTAAGGCGGTGGACTT
TATCCCCGTGGAGAACCTAGAGACAACCATGAGGTCCCCGGTGTTTACGGACAACTCCTCTCCGCCAGCAGTGCCCCAGAGCTTCCAGGTGGC
CCACCTGCATGCTCCCACGGGCAGCGGCAAGAGCACCAAGGTCCCAGCTGCATATGCGGCTCAGGGCTATAAAGTGCTAGTGCTCAACCCCTC
TGTTGCTGCAACACTGGGCTTTGGTGCTTATATGTCCAAGGCCCACGGGATTGATCCTAACATCAGGACCGGGGCGAGAACAATTACCACTGG
CAGCCCCATCACGTACTCCACCTACGGCAAGTTCCTTGCCGACGGCGGGTGCTCGGGGGGCGCCTATGATATAATAATTTGTGACGAGTGCCA
CTCCACGGATGCCACATCCATCTTGGGCATCGGCACTGTCCTTGACCAGGCAGAGACCGCGGGGGCGAGACTAGTTGTGCTCGCCACCGCCAC
CCCTCCGGGCTCCATCACTGTGCCCCATCCTAACATCGAGGAGGTTGCTCTGTCCACCACCGGAGAGATTCCTTTCTATGGCAAGGCTATCCC
CCTCGAAGTAATCAAAGGGGGGAGACATCTCATTTTTTGTCACTCAAAGAAGAAGTGCGATGAGCTCGCCGCAAAGCTGGTCGCATTGGGCAT
CAATGCCGTGGCCTACTACCGCGGTCTTGACGTGTCCGTCATCCCGACCAGCGGCGATGTTGTCGTCGTGGCAACTGATGCTCTCATGACCGG
TTTTACCGGCGACTTCGACTCGGTGATAGACTGCAACACGTGTGTCACCCAGACGGTCGATTTCAGCCTTGACCCTACCTTCACCATTGAGAC
AACCACGCTTCCCCAAGATGCTGTCTCCCGCACTCAGCGTCGGGGCAGGACTGGCAGGGGGAAGCCAGGCATCTACAGGTTTGTGGCACCAGG
GGAGCGCCCTTCCGGCATGTTCGACTCGTCCATCCTCTGTGAGTGCTATGACGCGGGTTGTGCTTGGTATGAGCTCACACCCGCCGAGACTAC
AGTTAGACTACGAGCGTACATGAACACCCCGGGGCTTCCCGTGTGCCAAGACCATCTCGAATTTTGGGAGGGGGTCTTCACAGGCCTCACTCA
TATAGATGCCCACTTCCTATCCCAGACAAAGCAGAGTGGGGAGAATCTTCCTTACCTGGTAGCGTACCAAGCCACCGTGTGCGCTAGGGCTCA
AGCCCCTCCCCCATCGTGGGACCAGATGTGGAAGTGTTTGATCCGCCTCAAACCCACCCTTCATGGGCCAACACCTCTGCTATACAGACTGGG
TGCTGTCCAGAATGAGGTCACCCTGACGCACCCAGTCACCAAATATATCATGA
Donor 67539/ Panel Number 10029/ Transmitted Variant #1 (SEQ ID NO: 8)
TCACTCCCCTGTGAGGAACTACTGTCTTCACGCAGAAAGCGTCTAGCCATGGCGTTAGTATGAGTGTCGTGCAGCCTCCAGGACCCCCCCTCC
CGGGAGAGCCATAGTGGTCTGCGGAACCGGTGAGTACACCGGAATTGCCAGGACGACCGGGTCCTTTCTTGGATAAACCCGCTCAATGCCTGG
AGATTTGGGCGTGCCCCCGCAAGACTGCTAGCCGAGTAGTGTTGGGTCGCGAAAGGCCTTGTGGTACTGCCTGATAGGGTGCTTGCGAGTGCC
CCGGGAGGTCTCGTAGACCGTGCACCATGAGCACGAATCCTAAACCTCAAAGAAAAACCAAACGTAACACCAACCGTCGCCCACAGGACGTCA
AGTTCCCGGGTGGCGGTCAGATCGTTGGTGGAGTTTACTTGTTGCCGCGCAGGGGCCCTAGATTGGGTGTGCGCGCGACGAGGAAGACTTCCG
AGCGGTCGCAACCTCGAGGTAGACGTCAGCCTATCCCCAAGGCACGTCGGCCCGAGGGCAGGACCTGGGCTCAGCCCGGGTACCCCTGGCCCC
TCTATGGCAATGAGGGCTGCGGGTGGGCGGGATGGCTCCTGTCTCCCCGTGGCTCTCGGCCTAACTGGGGCCCCACAGACCCCCGGCGTAGGT
CGCGTAATTTGGGTAAGGTCATCGATACCCTTACATGCGGCTTCGCCGACCTCATGGGGTACATACCGCTCGTCGGCGCCCCTCTTGGGGGCG
CTGCCAGGGCCCTGGCGCATGGCGTCCGGGTTCTGGAAGACGGCGTGAACTATGCAACAGGGAACCTTCCTGGTTGCTCTTTCTCTATCTTCC
TTCTGGCTTTGCTCTCTTGCCTGACTGTGCCTGCTTCAGCCTACCAAGTGCGCAACTCCACGGGGCTTTATCATGTCACCAATGATTGCCCTA
ACTCGAGTATTGTGTACGAGGCGGACGATGCTATCCTGCACACTCCGGGGTGTGTCCCTTGCGTCCGCGAGGGTAACGGCTCGAGGTGTTGGG
TGGCGATAACCCCCACGGTGGCCACCAGGGACGGCAAACTCCCCACAGTGCAGCTTCGACGTCACATCGATCTGCTCGTCGGGAGCGCTACCC
TTTGTTCGGCCCTTTACGTGGGGGACCTGTGCGGGTCTGTCTTTCTTGTCGGCCAACTGTTCACCTTCTCTCCCAGGCGCCACTGGACAACGC
AGGACTGCAATTGTTCCATCTATCCCGGCCATATAACGGGTCACCGTATGGCATGGGACATGATGATGAACTGGTCCCCTACGACGGCGCTGG
TAGTAGCTCAGATGCTCCGGATCCCACAAGCCATCCTGGACATGATCGCTGGTGCTCACTGGGGAGTCCTGGCGGGCATAGCGTATTTCTCCA
TGGTGGGGAACTGGGCGAAGGTCCTGGTAGTGCTGCTGCTATTTGCCGGCGTCGACGCGGGAACCCACGTCACCGGGGGCATTGCCGCCCGTA
CCACGTCTAGAATTGCTGGTCTCTTCAACCCAGGCGCCCAGCAGGACATCCAGCTGGTTAACACCAACGGCAGTTGGCACATCAACAGAACGG
CCTTGAACTGTAACGATAGCCTTCACACCGGCTGGATAGCGGGGCTTTTCTATCGCTACAAATTCAACTCTTCAGGCTGTCCCGAGAGGTTGG
CCAGCTGCCGACCCCTTACCGATTTTGCCCAGGGCTGGGGCCCTATTAGTTACGCCAACGGAAGCGGCCCCGACCAACGCCCCTACTGCTGGC
ACTATCCCCCAAAGCCTTGCGGCATTGTGCCCGCACAGAGCGTGTGTGGCCCGGTGTATTGCTTCACTCCCAGCCCCGTGGTGGTGGGAACGA
CCGACAAGGCGGGCGCGCCTACCTACAGATGGGGTGCAAACGATACGGACGTCTTCGTCCTTAACAACACCAGGCCACCACTGGGCAATTGGT
TCGGTTGCACCTGGATGAACTCAACTGGATACACCAAAGTGTGCGGGGCGCCCCCCTGCGTCATCGGAGGGGTGGGCAACAAGACCTTGCACT
GCCCCACCGATTGTTTCCGCAAGCATCCGGAAGCCACATACTCTCGGTGCGGCTCCGGTCCCTGGATTACACCCAGGTGTCTGGTCGACTATC
CGTATAGGCTTTGGCATTACCCTTGTACCATCAACTACACCGTGTTCAAAGTCAGGATGTACGTGGGAGGGGTCGAGCATAGGCTGGAGGCTG
CCTGCAACTGGACGCGGGGCGAACGTTGCAATCTGGAAGACAGGGACAGGTCCGAGCTCAGCCCGTTGCTGCTGTCCACCACACAGTGGCAAG
TCCTTCCGTGTTCCTTCACGACCCTGCCAGCCTTGTCCACCGGCCTCATCCACCTCCACCAGAACATTGTGGACGTGCAGTACTTATACGGGG
TGGGGTCAAGCATCGCGTCCTGGGCCATCAAGTGGGAGTACGTCGTTCTCCTATTCCTTCTGCTCGCAGACGCGCGCGTCTGCTCCTGCTTGT
GGATGATGTTACTCATATCCCAAGCGGAGGCGGCTTTGGAGAACCTCGTAGTGCTCAATGCAGCATCTTTGGCCGGGACACACGGTCTTGTGT
CCCTCCTCGTGTTCTTCTGCTTTGCGTGGTACCTGAAGGGTAGGTGGGTGCCCGGAGCGGTCTACGCCCTCTACGGGATGTGGCCTCTCCTCC
TGCTCCTATTGGCGTTGCCCCAGCGGGCATATGCGTTGGACACGGAGGTGGCCGCGTCGTGTGGCGGCGCTGTTCTTGTCGGGTTGATGGCGC
TGACTCTGTCACCATATTACAAGCGCTATGTCAGCTGGTGCTTATGGTGGCTTCAGTACTTTCTGACCAGAGCAGAAGCGCAACTGCACGTGT
GGGTTCCCCCTCTCGACGTCCGAGGGGGGCGCGACGCCGTCATCTTACTCATGTGTGCTGTACGCCCGGCTTTGGTGTTTGACATCACCAAGC
TGCTGCTGGCCGTCCTCGGACCCCTCTGGATTCTCCAAGCCAGTTTGCTTAAAGTGCCCTACTTTGTGCGCGTTCAAGGCCTTCTCCGGATCT
GCGCGCTAGCGCGGAAGATGGCGGGGGGTCATTACGTGCAAATGGTCATCATCAAGTTGGGGGCGCTCACTGGCACTTATGTGTATAACCATC
TCACTCCTCTTCGGGACTGGGCGCACAACGGCCTGCGAGATCTGGCTGTGGCTGTAGAGCCAGTCGTCTTCTCCCGAATGGAGACCAACCTCA
TCACGTGGGGGGCAGACACCGCCGCATGCGGCGACATCATCAACGGCTTGCCCGTCTCCGCCCGCAGGGGCCGGGAGATACTGCTCGGACCAG
CCGACGGAATGGTCTCCAAGGGGTGGAGGTTGCTGGCACCCATCACGGCATACGCCCAGCAGACAAGGGGCCTCCTAGGGTGTATAATCACCA
GCCTAACCGGCCGGGACAAAAATCAGGTGGAGGGTGAGGTCCAGATTGTGTCAACTGCTGCCCAAACCTTTCTGGCAACGTGCATCAATGGAG
TATGCTGGACCGTCTACCACGGGGCCGGAACGAGGACCATCGCATCACCCAAGGGTCCTGTTATCCAGATGTACACCAATGTAGATAAAGATC
TCGTGGGTTGGCCCGCTCCTCAAGGTTCCCGTTCTTTGGCACCCTGCACCTGCGGCTCCTCGGACCTTTATCTGGTCACGAGGCACGCCGATG
TCATTCCCGTGCGCCGGCGGGGTGATAGCAGGGGCAGCCTGCTTTCGCCCCGGCCCATTTCCTACTTGAAAGGCTCCTCGGGGGGTCCGCTGT
TGTGCCCCGCGGGGCACGCCGTGGGCATTTTCAGGGCCGCGGTGTGCACCCGTGGAGTGGCTAAGGCAGTGGACTTTATCCCTGTAGAGAACC
TAGAGACAACCATGAGGTCCCCGGTGTTCACGGACAACTCCTCTCCACCAGCAGTACCCCAGAGCTTCCAGGTGGCCCACCTGCATGCTCCCA
CAGGCAGCGGCAAGAGCACCAAGGTCCCAGCTGCATACGCAGCTCAGGGCTATAAGGTGCTAGTGCTCAACCCCTCTGTTGCTGCAACACTAG
GCTTCGGTGCTTATATGTCCAAGGCCCATGGGATCGACCCTAACATCAGGACCGGGGTGAGAACAATTACCACTGGCAGCCCCATCACGTACT
CCACCTACGGCAAGTTCCTTGCCGACGGCGGGTGCTCAGGGGGTGCTTATGACATAATAATCTGTGACGAGTGCCACTCCACGGATGCCACGT
CCATCTTAGGCATCGGCACCGTCCTTGACCAAGCAGAGACCGCAGGGGCGAGGCTTGTTGTGCTCGCCACCGCTACCCCTCCGGGCTCCATCA
CTGTGCCCCATCCTAACATCGAGGAGGTTGCTCTGTCCACCACCGGAGAGATCCCCTTTTATGGCAAGGCTATCCCCCTCGAAGTAATCAAGG
GGGGGAGGCATCTCATCTTCTGTCATTCAAAGAAGAAGTGTGACGAGCTCGCTGCAAAGCTGGTTGCATTGGGCGTCAATGCCGTGGCCTATT
ACCGCGGTCTTGACGTGTCTGTCATCCCGACCAGCGGCGATGTTGTCGTCGTGGCAACTGATGCTCTCATGACCGGCTATACCGGCGACTTCG
ACTCGGTGATAGACTGCAACACGTGTGTCACCCAGACAGTCGATTTCAGCCTTGACCCCACCTTCACCATTGAGACAACCACGCTTCCCCAGG
ATGCTGTCTCCCGTACACAACGGCGGGGCAGGACTGGCAGGGGGAAGCCAGGCACCTACAGGTTTGTGGCACCGGGGGAGCGCCCCTCTGGCA
TGTTCGACTCGTCTGTCCTCTGTGAGTGCTATGACGCGGGCTGTGCTTGGTATGAGCTCACGCCCGCCGAGACCACAGTTAGGCTACGAGCGT
ACATGAACACCCCGGGGCTTCCCGTGTGCCAGGACCACCTTGAATTTTGGGAGGGCGTCTTTACGGGCCTTACCCATATAGATGCCCACTTCC
TATCCCAGACAAAGCAGAGTGGGGAGAACCTTCCTTACCTGGTAGCGTACCAAGCCACCGTGTGCGCTAGGGCTCAAGCCCCTCCCCCGTCGT
GGGACCAGATGTGGAAGTGTTTAGTCCGCCTCAAGCCCACCCTCCATGGGCCAACACCCCTGCTATACAGATTGGGCGCTGTTCAGAATGAAG
TCACCCTGACACACCCAATCACAAAATACATCATGACATGTATGTCGGCTGA
Donor 67539/ Panel Number 10029/ Transmitted Variant #2 (SEQ ID NO: 9)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGTAGACGTCAGCCTATCCCCAAGGCACGTCGGCCCGAGGGCAGGACCTGGGCTCAGCC
CGGGTACCCCTGGCCCCTCTATGGCAATGAGGGCTGCGGGTGGGCGGGATGGCTCCTGTCTCCCCGTGGCTCTCGGCCTAACTGGGGCCCCAC
AGACCCCCGGCGTAGGTCGCGTAATTTGGGTAAGGTCATCGATACCCTTACATGCGGCTTCGCCGACCTCATGGGGTACATACCGCTCGTCGG
CGCCCCTCTTGGGGGCGCTGCCAGGGCCCTGGCGCATGGCGTCCGGGTTCTGGAAGACGGCGTGAACTATGCAACAGGGAACCTTCCTGGTTG
CTCTTTCTCTATCTTCCTTCTGGCTTTGCTCTCTTGCCTGACTGTGCCTGTTTCAGCCTACCAAGTGCGCAACTCCACGGGGCTTTATCATGT
CACCAATGATTGCCCTAATTCGAGTATTGTGTACGAGGCGGACGATGCTATCCTGCACACTCCGGGGTGTGTCCCTTGCGTCCGCGAGGGTAA
CGGCTCGAGGTGTTGGGTGGCGATAACCCCCACGGTGGCCACCAGGGACGGCAAACTCCCCACAGTGCAGCTTCGACGTCACATCGATCTGCT
TGTCGGGAGCGCTACCCTTTGTTCGGCCCTTTACGTGGGGGACCTGTGCGGGTCTGTCTTTCTTGTCGGCCAACTGTTCACCTTCTCTCCCAG
GCGCCACTGGACAACGCAGGACTGCAATTGTTCCATCTATCCCGGCCATATAACGGGTCACCGTATGGCATGGGACATGATGATGAACTGGTC
CCCTACGACGGCGCTGGTAGTAGCTCAGATGCTCCGGATCCCACAAGCCATCCTGGACATGATCGCTGGTGCTCACTGGGGAGTCCTGGCGGG
CATAGCGTATTTCTCCATGGTGGGGAACTGGGCGAAGGTCCTGGTAGTGCTGCTGCTATTTGCCGGCGTCGACGCGGGAACCCACGTCACCGG
GGGCATTGCCGCCCGTACCACGTCTAGAATTGCTGGTCTCTTCAACCCAGGCGCCCAGCAGGACATCCAGCTGGTTAACACCAACGGCAGTTG
GCACATCAACAGAACGGCCTTGAACTGTAACGATAGCCTTCACACCGGCTGGATAGCGGGGCTTTTCTATCGCTACAAATTCAACTCTTCAGG
CTGTCCCGAGAGGTTGGCCAGCTGCCGACCCCTTACCGATTTTGCCCAGGGCTGGGGCCCTATTAGTTACGCCAACGGAAGCGGCCCCGACCA
ACGCCCCTACTGCTGGCACTATCCCCCAAAGCCTTGCGGCATTGTGCCCGCACAGAGCGTGTGTGGCCCGGTGTATTGCTTCACTCCCAGCCC
CGTGGTGGTGGGAACGACCGACAAGGCGGGCGCGCCTACCTACAGATGGGGTGCAAACGATACGGACGTCTTCGTCCTTAACAACACCAGGCC
ACCACTGGGCAATTGGTTCGGTTGCACCTGGATGAACTCAACTGGATACACCAAAGTGTGCGGGGCGCCCCCCTGCGTCATCGGAGGGGTGGG
CAACAAGACCTTGCACTGCCCCACCGATTGTTTCCGCAAGCATCCGGAAGCCACATACTCTCGGTGCGGCTCCGGTCCCTGGATTACACCCAG
GTGCCTGGTCGACTATCCGTATAGGCTTTGGCATTACCCTTGTACCATCAACTACACCGTGTTCAAAGTCAGGATGTACGTGGGAGGGGTCGA
GCATAGGCTAGAGGCTGCCTGCAACTGGACGCGGGGCGAACGTTGCAATCTGGAAGACAGGGACAGGTCCGAGCTCAGCCCGTTGCTGCTGTC
CACCACACAGTGGCAAGTCCTTCCGTGTTCCTTCACGACCCTGCCAGCCTTGTCCACCGGCCTCATCCACCTCCACCAGAACATTGTGGACGT
GCAGTACTTATACGGGGTGGGGTCAAGCATCGCGTCCTGGGCCATCAAGTGGGAGTACGTCGTTCTCCTATTCCTTCTGCTCGCAGACGCGCG
CGTCTGCTCCTGCTTGTGGATGATGTTACTCATATCCCAAGCGGAGGCGGCTTTGGAGAACCTCGTAGTGCTCAATGCAGCATCTTTGGCCGG
GACACACGGTCTTGTGTCCCTCCTCGTGTTCTTCTGCTTTGCGTGGTACCTGAAGGGTAGGTGGGTGCCCGGAGCGGTCTACGCCCTCTACGG
GATGTGGCCTCTCCTCCTGCTCCTATTGGCGTTGCCCCAGCGGGCATATGCGTTGGACACGGAGGTGGCCGCGTCGTGTGGCGGCGCTGTTCT
TGTCGGGTTGATGGCGCTGACTCTGTCACCATATTACAAGCGCTATGTCAGCTGGTGCTTATGGTGGCTTCAGTACTTTCTGACCAGAGCAGA
AGCGCAACTGCACGTGTGGGTTCCCCCTCTCGACGTCCGAGGGGGGCGCGACGCCGTCATCTTACTCATGTGTGCTGTACGCCCGACTTTGGT
GTTTGACATCACCAAGCTGCTGCTGGCCGTCCTCGGACCCCTCTGGATTCTCCAAGCCAGTTTGCTTAAAGTGCCCTACTTTGTGCGCGTTCA
AGGCCTTCTCCGGATCTGCGCGCTAGCGCGGAAGATGGCGGGGGGTCATTACGTGCAAATGGTCATCATCAAGTTGGGGGCGCTCACTGGCAC
TTATGTGTATAACCATCTCACTCCTCTTCGGGACTGGGCGCACAACGGCCTGCGAGATCTGGCTGTGGCTGTAGAGCCAGTCGTCTTCTCCCG
AATGGAGACCAACCTCATCACGTGGGGGGCAGACACCGCCGCATGCGGCGACATCATCAACGGCTTGCCCGTCTCCGCCCGCAGGGGCCGGGA
GATACTGCTCGGACCAGCCGACGGAATGGTCTCCAAGGGGTGGAGGTTGCTGGCACCCATCACGGCGTACGCCCAGCAGACAAGGGGCCTCCT
AGGGTGTATAATCACCAGCCTAACCGGCCGGGACAAAAATCAGGTGGAGGGTGAGGTCCAGATTGTGTCAACTGCTGCCCAAACCTTTCTGGC
AACGTGCATCAATGGAGTATGCTGGACCGTCTACCACGGGGCCGGAACGAGGACCATCGCATCACCCAAGGGTCCGGTTATCCAGATGTACAC
CAATGTAGACAAAGATCTTGTGGGTTGGCCCGCTCCTCAAGGTTCCCGTTCTTTGGCACCCTGCACCTGCGGCTCCTCGGACCTTTATCTGGT
CACGAGGCACGCCGATGTCATTCCCGTGCGCCGGCGGGGTGATAGCAGGGGCAGCCTGCTTTCGCCCCGGCCCATTTCCTACTTGAAAGGCTC
CTCGGGGGGTCCGCTGTTGTGCCCCGCGGGGCACGCCGTGGGCATTTTCAGGGCCGCGGTGTGCACCCGTGGAGTGGCTAAGGCAGTGGACTT
TATCCCTGTAGAGAACCTAGAGACAACCATGAGGTCCCCGGTGTTCACGGACAACTCCTCTCCACCAGCAGTACCCCAGAGCTTCCAGGTGGC
CCACCTGCATGCTCCCACAGGCAGCGGCAAGAGCACCAAGGTCCCAGCTGCATACGCAGCTCAGGGCTATAAGGTGCTAGTGCTCAACCCCTC
TGTTGCTGCAACACTAGGCTTCGGTGCTTATATGTCCAAGGCCCATGGGATCGACCCTAACATCAGGACCGGGGTGAGAACAATTACCACTGG
CAGCCCCATCACGTACTCCACCTACGGCAAGTTCCTTGCCGACGGCGGGTGCTCAGGGGGTGCTTATGACATAATAATCTGTGACGAGTGCCA
CTCCACGGATGCCACGTCCATCTTAGGCATCGGCACCGTCCTTGACCAAGCAGAGACCGCAGGGGCGAGGCTTGTTGTGCTCGCCACCGCTAC
CCCTCCGGGCTCCATCACCGTGCCCCATCCTAACATCGAGGAGGTTGCTCTGTCCACCACCGGAGAGATCCCCTTTTATGGCAAGGCTATCCC
CCTCGAAGTAATCAAGGGGGGGAGGCATCTCATCTTCTGTCATTCAAAGAAGAAGTGTGACGAGCTCGCTGCAAAGCTGGTTGCATTGGGCGT
CAATGCCGTGGCCTATTACCGCGGTCTTGACGTGTCTGTCATCCCGACCAGCGGCGATGTTGTCGTCGTGGCAACTGATGCTCTCATGACCGG
CTATACCGGCGACTTCGACTCGGTGATAGACTGCAACACGTGTGTCACCCAGACAGTCGATTTCAGCCTTGACCCCACCTTCACCATTGAGAC
AACCACGCTTCCCCAGGATGCTGTCTCCCGTACACAACGGCGGGGCAGGACTGGCAGGGGGAAGCCAGGCACCTACAGGTTTGTGGCACCGGG
GGAGCGCCCCTCTGGCATGTTCGACTCGTCTGTCCTCTGTGAGTGCTATGACGCGGGCTGTGCTTGGTATGAGCTCACGCCCGCCGAGACCAC
AGTTAGGCTACGAGCGTACATGAACACCCCGGGGCTTCCCGTGTGCCAGGACCACCTTGAATTTTGGGAGGGCGTCTTTACGGGCCTTACCCA
TATAGATGCCCACTTCCTATCCCAGACAAAGCAGAGTGGGGAGAACCTTCCTTACCTGGTAGCGTACCAAGCCACCGTGTGCGCTAGGGCTCA
AGCCCCTCCCCCATCGTGGGACCAGATGTGGAAGTGTTTAGTCCGCCTCAAGCCCACCCTCCATGGGCCAACACCCCTGCTATACAGATTGGG
CGCTGTTCAGAATGAAGTCACCCTGACACACCCAATCACAAAATACATCATGACATGTATGTCGGCTGA
Donor 67539/ Panel Number 10029/ Transmitted Variant #3 (SEQ ID NO: 10)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGTAGACGTCAGCCTATCCCCAAGGCACGTCGGCCCGAGGGCAGGACCTGGGCTCAGCC
CGGGTACCCCTGGCCCCTCTATGGCAATGAAGGCTGCGGGTGGGCGGGATGGCTCCTGTCTCCCCGTGGCTCTCGGCCTAATTGGGGCCCCAC
AGACCCCCGGCGTAGGTCGCGTAATTTGGGTAAGGTCATCGATACCCTTACATGCGGCTTCGCCGACCTCATGGGGTACATACCGCTCGTCGG
CGCCCCTCTTGGGGGCGCTGCCAGGGCCCTGGCGCATGGCGTCCGGGTTCTGGAAGACGGCGTGAACTATGCAACAGGGAACCTTCCTGGTTG
CTCTTTCTCTATCTTCCTTCTGGCTTTGCTCTCTTGCCTGACTGTGCCTGCTTCAGCCTTCCAAGTGCGCAACTCCACGGGGCTTTATCATGT
CACCAATGATTGCCCTAACTCGAGTATTGTGTACGAGGCGGACGATGCTATCCTGCACACTCCGGGGTGTGTCCCTTGCGTCCGCGAGGGTAA
CGGCTCGAGGTGTTGGGTGGCGATAACCCCCACGGTGGCCACCAGGGACGGCAAACTCCCCACAGTGCAGCTTCGACGTCACATCGATCTGCT
TGTCGGGAGCGCTACCCTTTGTTCGGCCCTTTACGTGGGGGACCTGTGCGGGTCTGTCTTTCTTGTCGGCCAACTGTTCACCTTCTCTCCCAG
GCGCCACTGGACAACGCAGGACTGCAATTGTTCCATCTATCCCGGCCATATAACGGGTCACCGTATGGCATGGGACATGATGATGAACTGGTC
CCCTACGACGGCGCTGGTAGTAGCTCAGATGCTCCGGATCCCACAAGCCATCCTGGACATGATCGCTGGTGCTCACTGGGGAGTCCTGGCGGG
CATAGCGTATTTCTCCATGGTGGGGAACTGGGCGAAGGTCCTGGTAGTGCTGCTGCTATTTGCCGGCGTCGACGCGGGAACCCACGTCACCGG
GGGCATTGCCGCCCGTACCACGTCTGGAGTTGCTGGTCTCTTCAGCCCAGGCGCCCAGCAGGACATCCAGCTGGTTAACACCAACGGCAGTTG
GCACATCAACAGAACGGCCTTGAACTGTAACGATAGCCTTCACACCGGCTGGATAGCGGGGCTTTTCTATCGCTACAAATTCAACTCTTCAGG
CTGTCCCGAGAGGTTGGCCAGCTGCCGACCCCTTACCGATTTTGCCCAGGGCTGGGGCCCTATTAGTTACGCCAACGGAAGCGGCCCCGACCA
ACGCCCCTACTGCTGGCACTATCCCCCAAAGCCTTGCGGCATTGTGCCCGCACAGAGCGTGTGTGGCCCGGTGTATTGCTTCACTCCCAGCCC
CGTGGTGGTGGGAACGACCGACAAGGCGGGCGCGCCTACCTACAGATGGGGTGCAAACGATACGGACGTCTTCGTCCTTAACAACACCAGGCC
ACCACTGGGCAATTGGTTCGGTTGCACCTGGATGAACTCAACTGGATACACCAAAGTGTGCGGGGCGCCCCCCTGCGTCATCGGAGGGGTGGG
CAACAAGACCTTGCACTGCCCCACCGATTGTTTCCGCAAGCATCCGGAAGCCACATACTCTCGGTGCGGCTCCGGTCCCTGGATTACACCCAG
GTGTCTGGTCGACTATCCGTATAGGCTTTGGCATTACCCTTGTACCATCAACTACACCGTGTTCAAAGTCAGGATGTACGTGGGAGGGGTCGA
GCATAGGCTAGAGGCTGCCTGCAGCTGGACGCGGGGCGAACGTTGCAATCTGGAAGACAGGGACAGGTCCGAGCTCAGCCCGTTGCTGCTGTC
CACCACACAGTGGCAAGTCCTTCCGTGTTCCTTCACGACCCTGCCAGCCTTGTCCACCGGCCTCATCCACCTCCACCAGAACATTGTGGACGT
GCAGTACTTATACGGGGTGGGGTCAAGCATCGCGTCCTGGGCCATCAAGTGGGAGTACGTCGTTCTCCTATTCCTTCTGCTCGCAGACGCGCG
CGTCTGCTCCTGCTTGTGGATGATGTTACTCATATCCCAAGCGGAGGCGGCTTTGGAGAACCTCGTAGTGCTCAATGCAGCATCTTTGGCCGG
GACACACGGTCTTGTGTCCCTCCTCGTGTTCTTCTGCTTTGCGTGGTACCTGAAGGGTAGGTGGGTGCCCGGAGCGGTCTACGCCCTCTACGG
GATGTGGCCTCTCCTCCTGCTCCTATTGGCGTTGCCCCAGCGGGCATATGCGTTGGACACGGAGGTGGCCGCGTCGTGTGGCGGCGCTGTTCT
TGTCGGGTTGATGGCGCTGACTCTGTCACCATATTACAAGCGCTATGTCAGCTGGTGCTTATGGTGGCTTCAGTACTTTCTGACCAGAGCAGA
AGCGCAACTGCACGTGTGGGTTCCCCCTCTCGACGTCCGAGGGGGGCGCGACGCCGTCATCTTACTCATGTGTGCTGTACGCCCGACTTTGGT
GTTTGACATCACCAAGCTGCTGCTGGCCGTCCTCGGACCCCTCTGGATTCTCCAAGCCAGTTTGCTTAAAGTGCCCTACTTTGTGCGCGTTCA
AGGCCTTCTCCGGATCTGCGCGCTAGCGCGGAAGATGGCGGGGGGTCATTACGTGCAAATGGTCATCATCAAGTTGGGGGCGCTCACTGGCAC
TTATGTGTATAACCATCTCACTCCTCTTCGGGACTGGGCGCACAACGGCCTGCGAGATCTGGCTGTGGCTGTAGAGCCAGTCGTCTTCTCCCG
AATGGAGACCGACCTCATCACGTGGGGGGCAGACACCGCCGCATGCGGCGACGTCATCAACGGCTTGCCCGTCTCCGCCCGCAGGGGCCGGGA
GATACTGCTCGGACCAGCCGACGGAATGGTCTCCAAGGGGTGGAGGTTGCTGGCACCCATCACGGCATACGCCCAGCAGACAAGGGGCCTCCT
AGGGTGCATAATCACCAGCCTAACCGGCCGGGACAAAAATCAGGTGGAGGGTGAGGTCCAGATTGTGTCAACTGCTGCCCAAACCTTTCTGGC
GACGTGCATCAATGGAGTATGCTGGACCGTCTACCACGGGGCCGGAACGAGGACCATCGCATCACCCAAGGGTCCTGTTATCCAGATGTACAC
CAATGTAGACAAAGATCTTGTGGGTTGGCCCGCTCCTCAAGGTTCCCGTTCTTTGGCACCCTGCACCTGCGGCTCCTCGGACCTTTATCTGGT
CACGAGGCACGCCGATGTCATTCCCGTGCGCCGGCGGGGTGATAGCAGGGGCAGCCTGCTTTCGCCCCGGCCCATTTCCTACTTGAAAGGCTC
CTCGGGGGGTCCGCTGTTGTGCCCCGCGGGGCACGCCGTGGGCATTTTCAGGGCCGCGGTGTGCACCCGTGGAGTGGCTAAGGCAGTGGACTT
TATCCCTGTAGAGAACCTAGAGACAACCATGAGGTCCCCGGTGTTCACGGACAACTCCTCTCCACCAGCAGTACCCCAGAGCTTCCAGGTGGC
CCACCTGCATGCTCCCACAGGCAGCGGCAAGAGCACCAAGGTCCCAGCTGCATACGCAGCTCAGGGCTATAAGGTGCTAGTGCTCAACCCCTC
TGTTGCTGCAACACTAGGCTTCGGTGCTTATATGTCCAAGGCCCATGGGATCGACCCTAACATCAGGACCGGGGTGAGAACAATTACCACTGG
CAGCCCCATCACGTACTCCACCTACGGCAAGTTCCTTGCCGACGGCGGGTGCTCAGGGGGTGCTTATGACATAATAATCTGTGACGAGTGCCA
CTCCACGGATGCCACGTCCATCTTAGGCATCGGCACCGTCCTTGACCAAGCAGAGACCGCAGGGGCGAGGCTTGTTGTGCTCGCCACCGCTAC
CCCTCCGGGCTCCATCACCGTGCCCCATCCTAACATCGAGGAGGTTGCTCTGTCCACCACCGGAGAGATCCCCTTTTATGGCAAGGCTATCCC
CCTCGAAGTAATCAAGGGGGGGAGGCATCTCATCTTCTGTCATTCAAAGAAGAAGTGTGACGAGCTCGCTGCAAAGCTGGTTGCATTGGGCGT
CAATGCCGTGGCCTATTACCGCGGTCTCGACGTGTCTGTCATCCCGACCAGCGGCGATGTTGTCGTCGTGGCAACTGATGCTCTCATGACCGG
CTATACCGGCGACTTCGACTCGGTGATAGACTGCAACACGTGTGTCACCCAGACAGTCGATTTCAGCCTTGACCCCACCTTCACCATTGAGAC
AACCACGCTTCCCCAGGATGCTGTCTCCCGTACACAACGGCGGGGCAGGACTGGCAGGGGGAAGCCAGGCACCTACAGGTTTGTGGCACCGGG
GGAGCGCCCCTCTGGCATGTTCGACTCGTCTGTCCTCTGTGAGTGCTATGACGCGGGCTGTGCTTGGTATGAGCTCACGCCCGCCGAGACCAC
AGTTAGGCTACGAGCGTACATGAACACCCCGGGGCTTCCCGTGTGCCAGGACCACCTTGAATTTTGGGAGGGCGTCTTTACGGGCCTTACCCA
TATAGATGCCCACTTCCTATCCCAGACAAAGCAGAGTGGGGAGAACCTTCCTTACCTGGTAGCGTACCAAGCCACCGTGTGCGCTAGGGCTCA
AGCCCCTCCCCCATCGTGGGACCAGATGTGGAAGTGTTTAGTCCGCCTCAAGCCCACCCTCCATGGGCCAACACCCCTGCTATACAGATTGGG
CGCTGTTCAGAATGAAGTCACCCTGACACACCCAATCACAAAATACATCATGACATGTATGTCGGCTGA
Donor 67539/ Panel Number 10029/ Transmitted Variant #4 (SEQ ID NO: 11)
TCACTCCCCTGTGAGGAACTACTGTCTTCACGCAGAAAGCGCCTAGCCATGGCGTTAGTATGAGTGTCGTGCAGCCTCCAGGACCCCCCCTCC
CGGGAGAGCCATAGTGGTCTGCGGAACCGGTGAGTACACCGGAATTGCCAGGACGACCGGGTCCTTTCTTGGATAAACCCGCTCAATGCCTGG
AGATTTGGGCGTGCCCCCGCAAGACCGCTAGCCGAGTAGTGTTGGGTCGCGAAAGGCCTTGTGGTACTGCCTGATAGGGTGCTTGCGAGTGCC
CCGGGAGGTCTCGTAGACCGTGCACCATGAGCACGAATCCTAAACCTCAAAGAAAAACCAAACGTAACACCAACCGTCGCCCACAGGACGTCA
AGTTCCCGGGTGGCGGTCAGATCGTTGGTGGAGTTTACTTGTTGCCGCGCAGGGGCCCTAGATTGGGTGTGCGCGCGACGAGGAAGACTTCCG
AGCGGTCGCAACCTCGAGGCAGACGTCAGCCTATCCCCAAGGCACGTCGGCCCGAGGGCAGGACCTGGGCTCAGCCCGGGTACCCCTGGCCCC
TCTATGGCAATGAGGGCTGTGGGTGGGCGGGATGGCTCCTGTCTCCCCGTGGCTCTCGGCCTAGCTGGGGCCCCTCAGACCCCCGGCGTAGGT
CGCGTAATTTGGGTAAAGTCATCGATACCCTTACATGCGGCTTCGCCGACCTCATGGGGTACATACCGCTCGTCGGCGCCCCTCTTGGGGGCG
CTGCCAGGGCCCTGGCGCATGGCGTCCGGGTTCTGGAAGACGGCGTGAACTATGCAACAGGGAACCTTCCTGGTTGCTCTTTCTCTATCTTCC
TCCTGGCTTTGCTCTCTTGCCTGACTGTGCCTGCTTCAGCCTACCAAGTGCGCAACTCCACGGGGCTTTATCATGTCACCAATGATTGCCCTA
ACTCGAGTATTGTGTACGAGGCGGCCGATGCTATCCTGCACACTCCGGGGTGTGTCCCTTGCGTCTACGAGGGTAACGCCTCGAGGTGTTGGG
TGGCGATAACCCCCACGGTGGCCACCAGGGACGGCAAACTCCCCACAGTGCAGCTTCGACGTCACATCGATCTGCTTGTCGGGAGCGCCACCC
TTTGTTCGGCCCTTTACGTGGGGGACCTGTGCGGGTCTGTCTTTCTTGTCGGCCAACTGTTCACCTTCTCTCCCAGGCTCCACTGGACAACGC
AGGACTGCAATTGTTCCATCTACCCCGGCCATATAACGGGTCACCGTATGGCATGGGACATGATGATGAACTGGTCCCCTACGACGGCGCTGG
TAGTAGCTCAGATGCTCCGGATCCCGCAAGCCATCTTGGACATGATCGCTGGTGCTCACTGGGGAGTCCTAGCGGGCATAGCGTATTTCTCCA
TGGTGGGGAACTGGGCGAAGGTCCTAGTAGTGCTGCTGCTATTTGCCGGCGTCGACGCGGAAACCCACGTCACCGGGGGAGCTGCCGGCCGTA
CCATGGCTGGATTTACTGGTCTCTTTACACGAGGCGCCCAGCAGAGCATCCAGTTGGTTAACACCAACGGCAGTTGGCACATCAACAGAACGG
CTTTGAACTGCAACGATAGCCTTCAGACCGGCTGGATAGCGGGGCTTCTCTATCACAACAAATTCAACTCTTCAGGCTGTCCGGAGAGGTTGG
CCAGCTGCCGACCCCTTACCGATTTTGCCCAGGGCTGGGGCCCTATCAGTTACGCCAACGGAAGCGGCCCCGACCAACGCCCCTACTGCTGGC
ACTACCCCCCAAGGCCTTGCGGCATTGTGCCCGCACAGAGCGTATGTGGCCCGGTATATTGCTTCACTCCCAGTCCCGTGGTGGTGGGAACGA
CCGACAGGTCGGGCGCGCCTACCTACGGATGGGGTGCAAACGATACGGACGTCTTCGTCCTTAACAACACTAGGCCACCACTGGGCAATTGGT
TCGGTTGCACCTGGATGAACTCAACTGGATACACCAAAGTGTGCGGGGCGCCCCCCTGTGTCATCAGAGGGGTGGGCAACAACACCTTGCACT
GCCCCACCGATTGTTTCCGCAAGCATCCGGAAGCCACATACTCTCGGTGCGGCTCCGGTCCCTGGATTACACCCAGGTGCCTGGTCAACTACC
CGTATAGGCTTTGGCATTACCCTTGTACCATCAACTACACCGTATTCAAGGTCAGGATGTACGTGGGAGGGGTCGAGCATAGGCTAGAGGCTG
CCTGCAACTGGACGCGGGGCGAACGTTGCAGTCTGGAAGACAGGGACAGGTCCGAGCTCAGCCCGTTGCTGCTGTCCACCACACAGTGGCAGG
TCCTTCCCTGTTCCTTCACGACCCTGCCAGCCTTGTCCACCGGCCTCATCCACCTCCACCAGAACATTGTGGACGTGCAGTACTTATACGGGG
TGGGGTCAAGTATCGCGTCCTGGGCCATCAAGTGGGAGTACGTCGTTCTCCTATTCCTTCTGCTTGCAGACGCGCGCGTCTGCTCCTGCTTGT
GGATGATGTTGCTCATATCCCAAGCGGAGGCGGCCTTGGAGAACCTCGTAGTGCTCAATGCAGCATCTTTGGCCGGGACGCACGGTCTTGTGT
CCTTCCTCGTGTTCTTCTGCTTTGCGTGGTACCTGAAGGGTAGGTGGGTGCCCGGAGCGGCCTACGCCCTCTACGGGATGTGGCCTCTCCTCC
TGCTCCTGTTGGCGTTGCCCCAGCGGGCATACGCGTTGGACACGGAGGTGGCCGCGTCGTGTGGCGGCGCTGTTCTTGTCGGGTTGATGGCGC
TGACTCTGTCACCACATTACAAGCGATATGTCAGCTGGTGCTTGTGGTGGCTTCAGTACTTTCTGACCAGAGCAGAAGCGCAACTGCACGTGT
GGGTTCCCCCCCTCGACGTCCGAGGGGGGCGCGACGCCGTCATCTTACTCGTGTGTGCTGTACGCCCGACTTTGGTGTTTGACATCACCAAGC
TGCTGCTGGCCGTCTTCGGGCCCCTTTGGATTCTCCAAGCCAGTTTGCTTAAAGTACCCTACTTTGTGCGCGTTCAAGGCCTTCTCCGGATCT
GCGCGCTAGCGCGGAAGATGGCGGGGGGTCATTACGTGCAAATGGTCATCATCAAGCTAGGGGCGCTCACTGGCACCTATGTTTACAACCACC
TCACTCCTCTTCGGGACTGGGCGCACAACGGCCTGCGAGATCTGGCTGTGGCTGTAGAGCCAGTCGTCTTCTCCCGAATGGAGACCAACCTCA
TCACGTGGGGGGCAGACACCGCCGCGTGCGGTGACATCATCAACGGCTTGCCCGTCTCCGCCCGTAGGGGCCGGGAGATACTGCTCGGACCAG
CCGACGGAATGGTCTCCAAGGGGTGGAGGTTGCTGGCACCCATCACGGCATACGCCCAGCAGACAAGGGGCCTCCTAGGGTGTATAATCACCA
GCCTAACCGGCCGGGACAAAAACCAGGTGGAGGGTGAGGTCCAGATTGTGTCAACTGCTGCCCAAACCTTCCTGGCAACGTGCATCAATGGAG
TATGCTGGACCGTCTACCACGGGGCCGGGACGAGGACCATCGCATCACCCAAGGGTCCTGTCATCCAGATGTACACCAATGTAGACAAAGATC
TTGTGGGCTGGCCCGCTCCCCAAGGTTCCCGTTCATTGACACCCTGCACCTGCGGCTCCTCGGACCTTTATCTGGTCACGAGGCACGCCGATG
TCATTCCCGTGCGCCGGCGGGGTGATAGCAGGGGCAGCCTGCTTTCGCCCCGGCCCATTTCCTACTTGAAAGGCTCCTCGGGGGGCCCGCTGT
TGTGCCCCGCGGGGCATGCTGTGGGCATATTCAGGGCCGCAGTGTGCACCCGTGGAGTGGCTAAGGCGGTGGACTTTATCCCTGTAGAGAACC
TAGAGACAACCATGAGGTCCCCGGTGTTCACGGACAACTCCTCTCCACCGGCAGTACCCCAGAGCTTCCAGGTGGCCCACCTGCATGCTCCCA
CAGGCAGCGGCAAGAGCACCAAGGTCCCGGCTGCATACGCAGCTCAGGGCTATAAGGTGCTAGTGCTCAACCCTTCTGTTGCTGCAACACTAG
GCTTCGGTGTTTATATGTCCAAGGCCCATGGGATCGATCCTAACATCAGGACCGGGGTGAGAACAATTACCACTGGCAGCCCCATCACGTACT
CCACCTACGGCAAGTTCCTTGCCGACGGCGGGTGCTCAGGGGGTGCTTATGACATAATAATCTGTGACGAGTGCCACTCCACGGATGCCACAT
CTATCTTAGGCATCGGCACCGTCCTTGACCAAGCAGAGACCGCGGGGGCGAGGCTTGTTGTGCTCGCCACCGCTACCCCTCCGGGCTCTGTCA
CCGTGCCCCATCCTAACATCGAGGAGGTTGCTCTGTCCACCACCGGAGAGATCCCTTTTTATGGCAAGGCTATCCCCCTCGAAGTAATCAAGG
GGGGGAGGCATCTCATCTTCTGTCATTCAAAGAAGAAGTGTGACGAGCTCGCCGCAAAGCTGGTCGCATTGGGCGTCAATGCCGTGGCCTATT
ACCGCGGCCTTGACGTGTCTGTCATCCCGACCAGCGGCGATGTTGTCGTCGTGGCAACTGATGCTCTCATGACCGGCTATACCGGCGACTTCG
ACTCGGTGATAGACTGCAACACGTGTGTCACCCAGACAGTTGATTTCAGCCTTGACCCCACCTTCACCATTGAGACAACCACGCTTCCCCAGG
ATGCTGTCTCCCGTACACAACGTCGGGGCAGGACTGGCAGGGGGAAGCCAGGCACCTACAGATTTGTGGCACCGGGGGAGCGCCCCTCCGGCA
TGTTCGACTCGTCTGTCCTCTGTGAGTGCTATGACGCGGGCTGTGCTTGGTATGAGCTCACGCCCGCCGAGACTACAGTTAGGCTACGAGCGT
ACATGAACACCCCGGGGCTTCCCGTGTGCCAGGACCACCTTGAATTTTGGGAGGGCGTCTTTACGGGCCTTACTCATATAGATGCCCACTTCC
TATCCCAGACAAAGCAGAGTGGGGAGAACCTTCCTTACCTGGTAGCGTACCAAGCCACCGTGTGCGCTAGGGCTCAAGCCCCTCCCCCATCGT
GGGACCAGATGTGGAAGTGCTTGGTCCGCCTCAAGCCCACCCTCCATGGGCCAACACCCCTGCTATACAGATTGGGCGCTGTTCAGAATGAAG
TCACCCTGACACACCCAATCACAAAATACATCATGACATGTATGTCGGCTGA
Donor 67539/ Panel Number 10029/ Transmitted Variant #5 (SEQ ID NO: 12)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGCAGACGTCAGCCTATCCCCAAGGCACGTCGGCCCGAGGGCAGGACCTGGGCTCAGCC
CGGGTACCCCTGGCCCCTCTATGGCAATGAGGGCTGTGGGTGGGCGGGATGGCTCCTGTCTCCCCGTGGCTCTCGGCCTAGCTGGGGCCCCTC
AGACCCCCGGCGTAGGTCGCGTAATTTGGGTAAAGTCATCGATACCCTTACATGCGGCTTCGCCGACCTCATGGGGTACATACCGCTCGTCGG
CGCTCCTCTTGGGGGCGCTGCCAGGGCCCTGGCGCATGGCGTCCGGGTTCTGGAAGACGGCGTGAACTATGCAACAGGGAACCTTCCTGGTTG
CTCTTTCTCTATCTTCCTCCTGGCTTTGCTCTCTTGCCTGACTGTGCCTGCTTCAGCCTACCAAGTGCGCAACTCCACGGGGCTTTATCATGT
CACCAATGATTGCCCTAACTCGAGTATTGTGTACGAGGCGGCCGATGCTATCCTGCACACTCCGGGGTGTGTCCCTTGCGTCTACGAGGGTAA
CGCCTCGAGGTGTTGGGTGGCGATAACCCCCACGGTGGCCACCAGGGATGGCAAACTCCCCACAGTCCAGCTTCGACGTCACATCGATCTGCT
TGTCGGGAGCGCTACCCTTTGTTCGGCCCTTTACGTGGGGGATCTGTGCGGGTCTGTTTTTCTTGTCGGCCAACTGTTCACCTTCTCTCCCAG
GCTCCACTGGACAACGCAGGACTGCAATTGTTCCATCTACCCCGGCCATATAACGGGTCACCGTATGGCATGGGACATGATGATGAACTGGTC
CCCTACGACGGCGCTGGTAGTAGCTCAGATGCTCCGGATCCCGCAAGCCATCTTGGACATGATCGCTGGTGCTCACTGGGGAGTCCTAGCGGG
CATAGCGTATTTCTCCATGGTGGGGAACTGGGCGAAGGTCCTAGTAGTGCTGCTGCTATTTGCCGGCGTCGACGCGGAAACCCACGTCACCGG
GGGAGCTGCCGGCCGTACCATGGCTGGATTTACTGGTCTCTTTACACAAGGCGCCAAGCAGAGCATCCAGTTGGTTAACACCAACGGCAGTTG
GCACATCAACAGAACGGCTTTGAACTGCAACGATAGCCTTCAGACCGGCTGGATAGCGGGGCTTCTCTATCACAACAAATTCAACTCTTCAGG
CTGTCCCGAGAGGTTGGCCAGCTGCCGACCCCTTACCGATTTTGCCCAGGGCTGGGGCCCTATCAGTTACGCCAACGGAAGCGGCCCCGACCA
ACGCCCCTACTGCTGGCACTACCCCCCAAAGCCTTGCGGCATTGTGCCCGCACAGAGCGTATGTGGCCCGGTATATTGTTTCACTCCTAGCCC
CGTGGTGGTGGGAACGACCGACAGGTCGGGCGCGCCTACCTACGGATGGGGTGCAAACGATACGGACGTCTTCGTCCTTAACAACACCAGGCC
ACCACTGGGCAATTGGTTCGGTTGCACCTGGATGAACTCAACTGGATACACCAAAGTGTGCGGGGCGCCCCCCTGTGCCATCAGAGGGGTGGG
CAACAACACCTTGCACTGCCCCACCGATTGTTTCCGCAAGCATCCGGAAGCCACATACTCTCGGTGCGGCTCCGGTCCCTGGATTACACCCAG
GTGCCTGGTCAACTACCCGTATAGGCTTTGGCATTACCCTTGTACCATCAACTACACCGTGTTCAAGGTCAGGATGTACGTGGGAGGGGTCGA
GCATAGGCTAGAGGCTGCCTGCAACTGGACGCGGGGCGAACGTTGCAGTCTGGAAGACAGGGACAGGTCCGAGCTCAGCCCGTTGCTGCTGTC
CACCACACAGTGGCAGGTCCTTCCCTGTTCCTTCACGACCCTGCCAGCCTTGTCCACCGGCCTCATCCACCTCCACCAGAACATTGTGGACGT
GCAGTACTTATACGGGGTGGGGTCAAGTATCGCGTCCTGGGCCATCAAGTGGGAGTACGTCGTTCTCCTATTCCTTCTGCTTGCAGACGCGCG
CGTCTGCTCCTGCTTGTGGATGATGTTACTCATATCCCAAGCGGAGGCGGCCTTGGAGAACCTCGTAGTGCTCAATGCAGCATCTTTGGCCGG
GACGCATGGTCTTGTGTCCTTCCTCGTGTTCTTCTGCTTTGCGTGGTACTTGAAGGGTAGGTGGGTGCCCGGAGCGGCCTACGCCCTCTACGG
GATGTGGCCTCTCCTCCTGCTCCTGTTGGCGTTGCCCCAGCGGGCATACGCGTTGGACACGGAGGTGGCCGCGTCGTGTGGCGGCGCTGTTCT
TGTCGGGTTGATGGCGCTGACTCTGTCACCACATTACAAGCGATATGTCAGCTGGTGCTTGTGGTGGCTTCAGTACTTTCTGACCAGAGCAGA
AGCGCAACTGCACGTGTGGGTTCCCCCCCTCGACGTCCGAGGGGGGCGCGACGCCGTCATCTTACTCATGTGTGCTGTACGCCCGACTTTGGT
GTTTGACATCACCAAGCTGCTGCTGGCCGTCTTCGGGCCCCTTTGGATTCTCCAAGCCAGTTTGCTTAAAGTACCCTACTTTGTGCGCGTTCA
AGGCCTTCTCCGGATCTGCGCGCTAGCGCGGAAGATGGCGGGGGGTCATTACGTGCAAATGGTCATCATCAAACTAGGGGCGCTCACTGGCAC
CTATGTTTATAACCACCTCACTCCTCTTCGGGACTGGGCGCACAACGGCCTGCGAGATCTGGCTGTGGCTGTAGAGCCAGTCGTCTTCTCCCG
AATGGAGACCAACCTCATCACGTGGGGGGCAGACACCGCCGCGTGCGGTGACATCATCAACGGCTTGCCCGTCTCCGCCCGTAGGGGCCGGGA
GATACTGCTCGGACCAGCCGACGGAATGGTCTCCAAGGGGTGGAGGTTGCTGGCACCCATCACGGCATACGCCCAGCAGACAAGGGGCCTCCT
AGGGTGTATAATCACCAGCCTAACCGGCCGGGACAAAAACCAGGTGGAGGGTGAGGTCCAGATTGTGTCAACTGCTGCCCAAACTTTCCTGGC
AACGTGCATCAATGGAGTATGCTGGACCGTCTACCACGGGGCCGGGACGAGGACCATCGCATCACCCAAGGGTCCTGTCATCCAGATGTACAC
CAATGTAGACAAAGATCTTGTGGGCTGGCCCGCTCCCCAAGGTTCCCGTTCATTGACACCCTGCACCTGCGGCTCCTCGGACCTTTATCTGGT
CACGAGGCACGCCGATGTCATTCCCGTGCGCCGGCGGGGTGATAGCAGGGGCAGCCTGCTTTCGCCCCGGCCCATTTCCTACTTGAAAGGCTC
CTCGGGGGGTCCGCTGTTGTGCCCCGCGGGGCATGCTGTGGGCATATTCAGGGCCGCAGTGTGCACCCGTGGAGTGGCTAAGGCGGTGGACTT
TATCCCTGTAGAGAACCTAGAGACAACCATGAGGTCCCCGGTGTTCACGGACAACTCCTCTCCACCGGCAGTACCCCAGAGCTTCCAGGTGGC
CCACCTGCATGCTCCCACAGGCAGCGGCAAGAGCACCAAGGTCCCGGCTGCATACGCAGCTCAGGGCTATAAGGTGCTAGTGCTCAACCCCTC
TGTTGCTGCAACACTGGGCTTCGGTGTTTATATGTCCAAGGCCCATGGGATCGATCCTAACATCAGGACCGGGGTGAGAACAATTACCACTGG
CAGCCCCATCACGTACTCCACCTACGGCAAGTTCCTTGCCGACGGCGGGTGCTCAGGGGGTGCTTATGACATAATAATCTGTGACGAGTGCCA
CTCCACGGATGCCACATCTATCTTAGGCATCGGCACCGTCCTTGACCAAGCAGAGACCGCGGGGGCGAGGCTTGTTGTGCTCGCCACCGCTAC
CCCTCCGGGCTCCGTCACCGTGCCCCATCCTAACATCGAGGAGGTTGCTCTGTCCACCACCGGAGAGATCCCTTTTTATGGCAAGGCTATCCC
CCTCGAAGTAATCAAGGGGGGGAGGCATCTCATCTTCTGTCATTCAAAGAAGAAGTGTGACGAGCTCGCCGCAAAGCTGGTTGCATTGGGCGT
CAATGCCGTGGCCTATTACCGCGGCCTTGACGTGTCTGTCATCCCGACCAGCGGCGATGTTGTCGTCGTGGCAACTGATGCTCTCATGACCGG
CTATACCGGCGACTTCGACTCGGTGATAGACTGCAATACGTGTGTCACCCAGACAGTTGATTTCAGCCTTGACCCCACCTTCACCATTGAGAC
AACCACGCTTCCCCAGGATGCTGTCTCCCGTACACAACGTCGGGGCAGGACTGGCAGGGGGAAGCCAGGCACCTACAGATTTGTGGCACCGGG
GGAGCGCCCCTCCGGCATGTTCGACTCGTCTGTCCTCTGTGAGTGCTATGACGCGGGCTGTGCTTGGTATGAGCTCACGCCCGCCGAGACTAC
AGTTAGGCTACGAGCGTACATGAACACCCCGGGGCTTCCCGTGTGCCAGGACCACCTTGAATTTTGGGAGGGCGTCTTTACGGGCCTTACTCA
TATAGATGCCCACTTCCTATCCCAGACAAAGCAGAGTGGGGAGAACCTTCCTTACCTAGTAGCGTACCAAGCCACCGTGTGCGCTAGGGCTCA
AGCCCCTCCCCCATCGTGGGACCAGATGTGGAAGTGCTTGGTCCGCCTCAAGCCCACCCTCCATGGGCCAACACCCCTGCTATACAGATTGGG
CGCTGTTCAGAATGAAGTCACCCTGACACACCCAATCACAAAATACATCATGACATGTATGTCGGCTGA
Donor 67539/ Panel Number 10029/ Transmitted Variant #6 (SEQ ID NO: 13)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGCAGACGTCAGCCTATCCCCAAGGCACGTCGGCCCGAGGGCAGGACCTGGGCTCAGCC
CGGGTACCCCTGGCCCCTCTATGGCAATGAGGGCTGTGGGTGGGCGGGATGGCTCCTGTCTCCCCGTGGCTCTCGGCCTAGCTGGGGCCCCTC
AGACCCCCGGCGTAGGTCGCGTAATTTGGGTAAAGTCATCGATACCCTTACATGCGGCTTCGCCGACCTCATGGGGTACATACCGCTCGTCGG
CGCTCCTCTTGGGGGCGCTGCCAGGGCCCTGGCGCATGGCGTCCGGGTTCTGGAAGACGGCGTGAACTATGCAACAGGGAACCTTCCTGGTTG
CTCTTTCTCTATCTTCCTCCTGGCTTTGCTCTCTTGCCTGACTGTGCCTGCTTCAGCCTACCAAGTGCGCAACTCCACGGGGCTTTATCATGT
CACCAATGATTGCCCTAACTCGAGTATTGTGTACGAGGCGGCCGATGCTATCCTGCACACTCCGGGGTGTGTCCCTTGCGTCTACGAGGGTAA
CGCCTCGAGGTGTTGGGTGGCGATAACCCCCACGGTGGCCACCAGGGATGGCAAACTCCCCACAGTCCAGCTTCGACGTCACATCGATCTGCT
TGTCGGGAGCGCTACCCTTTGTTCGGCCCTTTACGTGGGGGACCTGTGCGGGTCTGTCTTTCTTGTCGGCCAACTGTTCACCTTCTCTCCCAG
GCTCCACTGGACAACGCAGGACTGCAATTGTTCCATCTACCCCGGCCATATAACGGGTCACCGTATGGCGTGGGACATGATGATGAACTGGTC
CCCTACGACGGCGCTGGTAGTAGCTCAGATGCTCCGGATCCCGCAAGCCATCTTGGACATGATCGCTGGTGCTCACTGGGGAGTCCTAGCGGG
CATAGCGTATTTCTCCATGGTGGGGAACTGGGCGAAGGTCCTAGTAGTGCTGCTGCTATTTGCCGGCGTCGACGCGGAAACCCACGTCACCGG
GGGAGCTGCCGGCCGTACCATGGCTGGATTTACTGGTCTCTTTACACAAGGCGCCAAGCAGAGCATCCAGTTGGTTAACACCAACGGCAGTTG
GCACATCAACAGAACGGCTTTGAACTGCAACGATAGCCTTCAGACCGGCTGGATAGCGGGGCTTCTCTATCACAACAAATTCAACTCTTCAGG
CTGTCCCGAGAGGTTGGCCAGCTGCCGACCCCTTACCGATTTTGCCCAGGGCTGGGGCCCTATCAGCTACGCCAACGGAAGCGGCCCCGACCA
ACGCCCCTACTGCTGGCACTACCCCCCAAAGCCTTGCGGCATTGTGCCCGCACAGAGCGTATGTGGCCCGGTATATTGTTTCACTCCTAGCCC
CGTGGTGGTGGGAACGACCGACAGGTCGGGCGCGCCTACCTACGGATGGGGTGCAAACGATACGGACGTCTTCGTCCTTAACAACACCAGGCC
ACCACTGGGCAATTGGTTCGGTTGCACCTGGATGAACTCAACTGGATACACCAAAGTGTGCGGGGCGCCCCCCTGTGCCATCAGAGGGGTGGG
CAACAACACCTTGCACTGCCCCACCGATTGTTTCCGCAAGCATCCGGAAGCCACATACTCTCGGTGCGGCTCCGGTCCCTGGATTACACCCAG
GTGCCTGGTCAACTACCCGTATAGGCTTTGGCATTACCCTTGTACCATCAACTACACCGTGTTCAAGGTCAGGATGTACGTGGGAGGGGTCGA
GCACAGGCTAGAGGCTGCCTGCAACTGGACGCGGGGCGAACGTTGCAGTCTGGAAGACAGGGACAGGTCCGAGCTCAGCCCGTTGCTGCTGTC
CACCACACAGTGGCAGGTCCTTCCCTGTTCCTTCACGACCCTGCCAGCCTTGTCCACCGGCCTCATCCACCTCCACCAGAACATTGTGGACGT
GCAGTACTTATACGGGGTGGGGTCAAGTATCGCGTCCTGGGCCATCAAGTGGGAGTACGTCGTTCTCCTATTCCTTCTGCTTGCAGACGCGCG
CGTCTGCTCCTGCTTGTGGATGATGTTACTCATATCCCAAGCGGAGGCGGCCTTGGAGAACCTCGTAGTGCTCAATGCAGCATCTTTGGCCGG
GACGCATGGTCTTGTGTCCTTCCTCGTGTTCTTCTGCTTTGCGTGGTACCTGAAGGGTAGGTGGGTGCCCGGAGCGGCCTACGCCCTCTACGG
GATGTGGCCTCTCCTCCTGCTCCTGTTGGCGTTGCCCCAGCGGGCATACGCGTTGGACACGGAGGTGGCCGCGTCGTGTGGCGGCGCTGTTCT
TGTCGGGTTGATGGCGCTGACTCTGTCACCACATTACAAGCGATATGTCAGCTGGTGCTTGTGGTGGCTTCAGTACTTTCTGACCAGAGCAGA
AGCGCAACTACACGTGTGGGTTCCCCCCCTCGACGTCCGAGGGGGGCGCGACGCCGTCATCTTACTCATGTGTGCTGTACGCCCGACTTTGGT
GTTTGACATCACCAAGCTGCTGCTGGCCGTCTTCGGGCCCCTTTGGATTCTCCAGGCCAGTTTGCTTAAAGTACCCTACTTTGTGCGCGTTCA
AGGCCTTCTCCGGATCTGCGCGCTAGCGCGGAAGATGGCGGGGGGTCATTACGTGCAAATGGTCATCATCAAACTAGGGGCGCTCACTGGCAC
CTATGTTTATAACCACCTCACTCCTCTTCGGGACTGGGCGCACAACGGCCTGCGAGATCTGGCTGTGGCTGTAGAGCCAGTCGTCTTCTCCCG
AATGGAGACCAACCTCATCACGTGGGGGGCAGACACCGCCGCGTGCGGTGACATCATCAACGGCTTGCCCGTCTCCGCCCGTAGGGGCCGGGA
GATACTGCTCGGACCAGCCGACGGAATGGTCTCCAAGGGGTGGAGGTTGCTGGCACCCATCACGGCATACGCCCAGCAGACAAGGGGCCTCCT
AGGGTGTATAATCACCAGCCTAACCGGCCGGGACAAAAACCAGGTGGAGGGTGAGGTCCAGATTGTGTCAACTGCTGCCCAAACTTTCCTGGC
AACGTGCATCAATGGAGTATGCTGGACCGTCTACCACGGGGCCGGGACGAGGACCATCGCATCACCCAAGGGTCCTGTCATCCAGATGTACAC
CAATGTAGACAAAGATCTTGTGGGCTGGCCCGCTCCCCAAGGTTCCCGTTCATTGACACCCTGCACCTGTGGCTCCTCGGACCTTTATCTGGT
CACGAGGCACGCCGATGTCATTCCCGTGCGCCGGCGGGGTGATAGCAGGGGCAGCCTGCTTTCGCCCCGGCCCATTTCCTACTTGAAAGGCTC
CTCGGGGGGTCCGCTGTTGTGCCCCGCGGGGCATGCTGTGGGCATATTCAGGGCCGCAGTGTGCACCCGTGGAGTGGCTAAGGCGGTGGACTT
TATCCCTGTAGAGAACCTAGAGACAACCATGAGGTCCCCGGTGTTCACGGACAACTCCTCTCCACCGGCAGTACCCCAGAGCTTCCAGGTGGC
CCACCTGCATGCTCCCACAGGCAGCGGCAAGAGCACCAAGGTCCCGGCTGCATATGCAGCTCAGGGCTATAAGGTGCTAGTGCTCAACCCCTC
TGTTGCTGCAACACTGGGCTTCGGTGTTTATATGTCCAAGGCCCATGGGATCGATCCTAACATCAGGACCGGGGTGAGAACAATTACCACTGG
CAGCCCCATCACGTACTCCACCTACGGCAAGTTCCTTGCCGACGGCGGGTGCTCAGGGGGTGCTTATGACATAATAATCTGTGACGAGTGCCA
CTCCACGGATGCCACATCTATCTTAGGCATCGGCACCGTCCTTGACCAAGCAGAGACCGCGGGGGCGAGGCTTGTTGTGCTCGCCACCGCTAC
TCCTCCGGGCTCCGTCACCGTGCCCCATCCTAACATCGAGGAGGTTGCTCTGTCCACCACCGGAGAGATCCCTTTTTATGGCAAGGCTATCCC
CCTCGAAGTAATCAAGGGGGGGAGGCATCTCATCTTCTGTCATTCAAAGAAGAAGTGTGACGAGCTCGCCGCAAAGCTGGTTGCATTGGGCGT
CAATGCCGTGGCCTATTACCGCGGCCTTGACGTGTCTGTCATCCCGACCAGCGGCGATGTTGTCGTCGTGGCAACTGATGCTCTCATGACCGG
CTATACCGGCGACTTCGACTCGGTGATAGACTGCAATACGTGTGTCACCCAGACAGTTGATTTCAGCCTTGACCCCACCTTCACCATTGAGAC
AACCACGCTTCCCCAGGATGCTGTCTCCCGTACACAACGTCGGGGCAGGACTGGCAGGGGGAAGCCAGGCACCTACAGATTTGTGGCACCGGG
GGAGCGCCCCTCCGGCATGTTCGACTCGTCTGTCCTCTGTGAGTGCTATGACGCGGGCTGTGCTTGGTATGAGCTCACGCCCGCCGAGACTAC
AGTTAGGCTACGAGCGTACATGAACACCCCGGGGCTTCCCGTGTGCCAGGACCACCTTGAATTTTGGGAGGGCGTCTTTACGGGCCTTACTCA
TATAGATGCCCACTTCCTATCCCAGACAAAGCAGAGTGGGGAGAACCTTCCTTACCTGGTAGCGTACCAAGCCACCGTGTGCGCTAGGGCTCA
AGCCCCTCCCCCATCGTGGGACCAGATGTGGAAGTGCTTGGTCCGCCTCAAGCCCACCCTCCATGGGCCAACACCCCTGCTATACAGATTGGG
CGCTGTTCAGAATGAAGTCACCCTGACACACCCAATCACAAAATACATCATGA
Donor 67539/ Panel Number 10029/ Transmitted Variant #7 (SEQ ID NO: 14)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGCAGACGTCAGCCTATCCCCAAGGCACGTCGGCCCGAGGGCAGGACCTGGGCTCAGCC
CGGGTACCCCTGGCCCCTCTATGGCAATGAGGGCTGTGGGTGGGCGGGATGGCTCCTGTCTCCCCGTGGCTCTCGGCCTAGCTGGGGCCCCTC
AGACCCCCGGCGTAGGTCGCGTAATTTGGGTAAAGTCATCGATACCCTTACATGCGGCTTCGCCGACCTCATGGGGTACATACCGCTCGTCGG
CGCTCCTCTTGGGGGCGCTGCCAGGGCCCTGGCGCATGGCGTCCGGGTTCTGGAAGACGGCGTGAACTATGCAACAGGGAACCTTCCTGGTTG
CTCTTTCTCTATCTTCCTCCTGGCTTTGCTCTCTTGCCTGACTGTGCCTGCTTCAGCCTACCAAGTGCGCAACTCCACGGGGCTTTATCATGT
CACCAATGATTGCCCTAACTCGAGTATTGTGTACGAGGCGGCCGATGCTATCCTGCACACTCCGGGGTGTGTCCCTTGCGTCTACGAGGGTAA
CGCCTCGAGGTGTTGGGTGGCGATAACCCCCACGGTGGCCACCAGGGATGGCAAACTCCCCACAGTCCAGCTTCGACGTCACATCGATCTGCT
TGTCGGGAGCGCTACCCTTTGTTCGGCCCTTTACGTGGGGGACCTGTGCGGGTCTGTCTTTCTTGTCGGCCAACTATTCACCTTCTCTCCCAG
GCTCCACTGGACAACGCAGGACTGCAATTGTTCCATCTACCCCGGCCATATAACGGGTCACCGTATGGCATGGGACATGATGATGAACTGGTC
CCCTACGACGGCGCTGGTAGTAGCTCAGATGCTCCGGATCCCGCAAGCCATCTTGGACATGATCGCTGGTGCTCACTGGGGAGTCCTAGCGGG
CATAGCGTATTTCTCCATGGTGGGGAACTGGGCGAAGGTCCTAGTAGTGCTGCTGCTATTTGCCGGCGTCGACGCGGAAACCCACGTCACCGG
GGGAGCTGCCGGCCGTACCATGGCTGGATTTACTGGTCTCTTTACACAAGGCGCCAAGCAGAGCATCCAGTTGGTTAACACCAACGGCAGTTG
GCACATCAACAGAACGGCTTTGAACTGCAACGATAGCCTTCAGACCGGCTGGATAGCGGGGCTTCTCTATCACAACAAATTCAACTCTTCAGG
CTGTCCCGAGAGGTTGGCCAGCTGCCGACCCCTTACCGATTTTGCCCAGGGCTGGGGCCCTATCAGTTACGCCAACGGAAGCGGGCCCGACCA
ACGCCCCTACTGCTGGCACTACCCCCCAAAGCCTTGCGGCATTGTGCCCGCACAGAGCGTATGTGGCCCGGTATATTGTTTCACTCCTAGCCC
CGTGGTGGTGGGAACGACCGACAGGTCGGGCGCGCCTACCTACGGATGGGGTGCAAACGATACGGACGTCTTCGTCCTTAACAACACCAGGCC
ACCACTGGGCAATTGGTTCGGTTGCACCTGGATGAACTCAACTGGATACACCAAAGTGTGCGGGGCGCCCCCCTGTGTCATCAGAGGGGTGGG
CAATAACACCTTGCACTGCCCCACCGATTGTTTCCGCAAGCATCCGGAAACCACATACTCTCGGTGCGGCTCCGGTCCCTGGATTACACCCAG
GTGCCTGGTCAACTACCCGTATAGGCTTTGGCATTACCCTTGTACCATCAACTACACCGTGTTCAAGGTCAGGATGTACGTGGGAGGGGTCGA
GCATAGGCTAGAGGCTGCCTGCAACTGGACGCGGGGCGAACGTTGCAGTCTGGAAGACAGGGACAGGTCCGAGCTCAGCCCGTTGCTGCTGTC
CACCACACAGTGGCAGGTCCTTCCCTGTTCCTTCACGACCCTGCCAGCCTTGTCCACCGGCCTCATCCACCTCCACCAGAACATTGTGGACGT
GCAGTACTTATACGGGGTGGGGTCAAGTATCGCGTCCTGGGCCATCAAGTGGGAGTACGTCGTTCTCCTATTCCTTCTGCTTGCAGACGCGCG
CGTCTGCTCCTGCTTGTGGATGATGTTACTCATATCCCAAGCGGAGGCGGCCTTGGAGAACCTCGTAGTGCTCAATGCAGCATCTTTGGCCGG
GACGCATGGTCTTGTGTCCTTCCTCGTGTTCTTCTGCTTTGCGTGGTACCTGAAGGGTAGGTGGGTGCCCGGAGCGACCTACGCCCTCTACGG
GATGTGGCCTCTCCTCCTGCTCCTGTTGGCGTTGCCCCAGCGGGCATACGCGTTGGACACGGAGGTGGCCGCGTCGTGTGGCGGCGCTGTTCT
TGTCGGGTTGATGGCGCTGACTCTGTCACCACATTACAAGCGATATGTCAGCTGGTGCTTGTGGTGGCTTCAGTACTTTCTGACCAGAGCAGA
AGCGCAACTGCACGTGTGGGTTCCCCCCCTCGACGTCCGAGGGGGGCGCGACGCCGTCATCTTACTCATGTGTGCTGTACGCCCGACTTTGGT
GTTTGACATCACCAAGCTGCTGCTGGCCGTCTTCGGGCCCCTTTGGATTCTCCAAGCCAGTTTGCTTAAAGTACCCTACTTTGTGCGCGTTCA
AGGCCTTCTCCGGATCTGCGCGCTAGCGCGGAAGATGGCGGGGGGTCATTACGTGCAAATGGTCATCATCAAACTAGGGGCGCTCACTGGCAC
CTATGTTTATAACCACCTCACTCCTCTTCGGGACTGGGCGCACAACGGCCTGCGAGATCTGGCTGTGGCTGTAGAGCCAGTCGTCTTCTCCCG
AATGGAGACCAACCTCATCACGTGGGGGGCAGACACCGCCGCGTGCGGTGACATCATCAACGGCTTGCCCGTCTCCGCCCGTAGGGGCCGGGA
GATACTGCTCGGACCAGCCGACGGAATGGTCTCCAAGGGGTGGAGGTTGCTGGCACCCATCACGGCATACGCCCAGCAGACAAGGGGCCTCCT
AGGGTGTATAATCACCAGCCTAACCGGCCGGGACAAAAACCAGGTGGAGGGTGAGGTCCAGATTGTGTCAACTGCTGCCCAAACTTTCCTGGC
AACGTGCATCAACGGAGTATGCTGGACCGTCTACCACGGGGCCGGGACGAGGACCATCGCATCACCCAAGGGTCCTGTCATCCAGATGTACAC
CAATGTAGACAAAGATCTTGTGGGCTGGCCCGCTCCCCAAGGCTCCCGTTCATTGACACCCTGCACCTGCGGCTCCTCGGACCTTTATCTGGT
CACGAGGCACGCCGATGTCATTCCCGTGCGCCGGCGGGGTGATAGCAGGGGCAGCCTGCTTTCGCCCCGGCCTATTTCCTACTTGAAAGGCTC
CTCGGGGGGTCCGCTGTTGTGCCCCGCGGGGCATGCTGTGGGCATATTCAGGGCCGCAGTGTGCACCCGTGGAGTGGCTAAGGCGGTGGACTT
TATCCCTGTAGAGAACCTAGAGACAACCATGAGGTCCCCGGTGTTCACGGACAACTCCTCTCCACCGGCAGTACCCCAGAGCTTCCAGGTGGC
CCACCTGCATGCTCCCACAGGCAGCGGCAAGAGCACCAAGGTCCCGGCTGCATACGCAGCTCAGGGCTATAAGGTGCTAGTGCTCAACCCCTC
TGTTGCTGCAACACTGGGCTTCGGTGTTTATATGTCCAAGGCCCATGGGATCGATCCTAACATCAGGACCGGGGTGAGAACAATTACCACTGG
CAGCCCCATCACGTACTCCACCTACGGCAAGTTCCTTGCCGACGGCGGGTGCTCAGGGGGTGCTTATGACATAATAATCTGTGACGAGTGCCA
CTCCACGGATGCCACATCTATCTTAGGCATCGGCACCGTCCTTGACCAAGCAGAGACCGCGGGGGCGAGGCTTGTTGTGCTCGCCACCGCTAC
CCCTCCGGGCTCCGTCACCGTGCCCCATCCTAACATCGAGGAGGTTGCTCTGTCCACCACCGGAGAGATCCCTTTTTATGGCAAGGCTATCCC
CCTCGAAGTAATCAAGGGGGGGAGGCATCTCATCTTCTGTCATTCAAAGAAGAAGTGTGACGAGCTCGCCGCAAAGCTGGTTGCATTGGGCGT
CAATGCCGTGGCCTATTACCGCGGCCTTGACGTGTCTGTCATCCCGACCAGCGGCGATGTTGTCGTCGTGGCAACTGATGCTCTCATGACCGG
CTATACCGGCGACTTCGACTCGGTGATAGACTGCAATACGTGTGTCACCCAGACAGTTGATTTCAGCCTTGACCCCACCTTCACCATTGAGAC
AACCACGCTTCCCCAGGATGCTGTCTCCCGTACACAACGTCGGGGCAGGACTGGCAGGGGGAAGCCAGGCACCTACAGATTTGTGGCACCGGG
GGAGCGCCCCTCCGGCATGTTCGACTCGTCTGTCCTCTGTGAGTGCTATGACGCGGGCTGTGCTTGGTATGAGCTCACGCCCGCCGAGACTAC
AGTTAGGCTACGAGCGTACATGAACACCCCGGGGCTTCCCGTGTGCCAGGACCACCTTGAATTTTGGGAGGGCGTCTTTACGGGCCTTACTCA
TATAGATGCCCACTTCCTATCCCAGACAAAGCAGAGTGGGGAGAACCTTCCTTACCTGGTAGCGTACCAAGCCACCGTGTGCGCTAGGGCTCA
AGCCCCTCCCCCATCGTGGGACCAGATGTGGAAGTGCTTGGTCCGCCTCAAGCCCACCCTCCATGGGCCAACACCCCTGCTATACAGATTGGG
CGCTGTTCAGAATGAAGTCACCCTGACACACCCAATCACAAAATACATCATGACATGTATGTCGGCTGA
Donor 67539/ Panel Number 10029/ Transmitted Variant #8 (SEQ ID NO: 15)
TCGCCCACAGGACGTCAAGTTCCCGGGTGGCGGTCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGCAGACGTCAGCCTATCCCCAAGGCACGTCGGCCCGAGGGTAGGACCTGGGCTCAGCC
CGGGTACCCCTGGCCCCTCTATGGCAATGAGGGCTGTGGGTGGGCGGGATGGCTCCTGTCTCCCCGTGGCTCTCGGCCTAGCTGGGGCCCCTC
AGACCCCCGGCGTAGGTCGCGTAATTTGGGTAAAGTCATCGATACCCTTACATGCGGCTTCGCCGACCTCATGGGGTACATACCGCTCGTCGG
CGCCCCTCTTGGGGGCGCTGCCAGGGCCCTGGCGCATGGCGTCCGGGTTCTGGAAGACGGCGTGAACTATGCAACAGGGAACCTTCCTGGTTG
CTCTTTCTCTATCTTCCTCCTGGCTTTGCTCTCTTGCCTGACTGTGCCTGCTTCAGCCTACCAAGTGCGCAACTCCACGGGGCTTTATCATGT
CACCAATGATTGCCCTAACTCGAGTATTGTGTACGAGGCGGCCGATGCTATCCTGCACACTCCGGGGTGTGTCCCTTGCGTCTACGAGGGTAA
CGCCTCGAGGTGTTGGGTGGCGATAACCCCCACGGTGGCCACCAGGGACGGCAAACTCCCCACAGTGCAGCTTCGACGTCACATCGATCTGCT
TGTCGGGAGCGCTACCCTTTGTTCGGCCCTTTACGTGGGGGACCTGTGCGGGTCTGTCTTTCTTGTCGGCCAACTGTTCACCTTCTCTCCCAG
GCTCCACTGGACAACGCAGGACTGTAATTGTTCCATCTACCCCGGCCATATAACGGGTCACCGTATGGCATGGGACATGATGATGAACTGGTC
CCCTACGACGGCGCTGGTAGTAGCTCAGATGCTCCGGATCCCGCAAGCCATCTTGGACATGATCGCTGGTGCTCACTGGGGAGTCCTAGCGGG
CATAGCGTATTTCTCCATGGTGGGGAACTGGGCGAAGGTCCTAGTAGTGCTGCTGCTATTTGCCGGCGTCGACGCGGGGACCCACGTCACCGG
GGGAGCTGCCGGCCGTACCACGGCTGGATTTGCTGGTCTCTTTACACGAGGCGCCCAGCAGAGCATCCAGTTGGTTAACACCAACGGCAGTTG
GCACATCAACAGAACGGCTTTGAACTGCAACGATAGCCTTCAGACCGGCTGGATAGCGGGGCTTCTCTATCACAACAAATTCAACTCTTCAGG
CTGTCCCGAGAGGTTGGCCAGCTGCCGACCCCTTACCGATTTTGCCCAGGGCTGGGGCCCTATCAGTTACGCCAACGGAAGCGGCCCCGACCA
ACGCCCCTACTGCTGGCACTACCCCCCAAAGCCTTGCGGCATTGTGCCCGCACAGAGCGTATGTGGCCCGGTATATTGTTTCACCCCTAGCCC
CGTGGTGGTGGGAACGACCGACAGGTCGGGCGCGCCTACCTACGGATGGGGTGCAAACGATACGGACGTCTTCGTCCTCAACAACACCAGGCC
ACCACTGGGCAATTGGTTCGGTTGCACCTGGATGAACTCAACTGGATATACCAAAGTGTGCGGGGCGCCCCCCTGTTTCATCAGAGGGGTGGG
CAACAACACCTTGCACTGCCCCACCGATTGCTTCCGCAAGCATCCGGAAGCCACATACTCTCGGTGCGGCTCCGGTCCCTGGATTACACCCAG
GTGCCTGGTCAACTACCCGTATAGGCTTTGGCATTACCCTTGTACCATCAACTACACCGTGTTCAAGGTCAGGATGTACGTGGGAGGGGTTGA
GCATAGGCTAGAGGCTGCCTGCAACTGGACGCGGGGCGAACGTTGCAGTCTGGAAGACAGGGACAGGTCCGAGCTCAGCCCGTTGCTGCTGTC
CACCACACAGTGGCAGGTCCTTCCCTGTTCCTTCACGACCCTGCCAGCCTTGTCCACTGGCCTCATCCACCTCCACCAGAACATTGTGGACGT
GCAGTACTTATACGGGGTGGGGTCAAGTATCGCGTCCTGGGCCATCAAGTGGGAGTACGTCGTTCTCCTATTCCTTCTGCTTGCAGACGCGCG
CGTCTGCTCCTGCTTGTGGATGATGTTGCTCATATCCCAAGCGGAGGCGGCCTTGGAGAACCTCGTAGTGCTCAATGCAGCATCTTTGGCCGG
GACGCATGGGTTTGTGTCCTTCCTCGTGTTCTTCTGCTTTGCGTGGTACCTGAAGGGTAGGTGGGTGCCCGGAGCAGCCTACGCCCTCTACGG
GATGTGGCCTCTCCTCCTGCTCCTGTTGGCGTTGCCCCAGCGGGCATACGCGTTGGACACGGAGGTGGCCGCGTCGTGTGGCGGCGCTGTTCT
TGTCGGGTTGATGGCGCTGACTCTGTCACCACATTACAAGCGATATGTCAGCTGGTGCTTGTGGTGGCTTCAGTACTTTCTGACCAGAGCAGA
AGCGCAACTGCACGTGTGGGTTCCCCCCCTCGACGTCCGAGGGGGGCGCGACGCCGTCATCTTACTCATGTGTGCTGTACGCCCGACTTTGGT
GTTTGACATCACCAAGCTGCTGCTGGCCGTCTTCGGGCCCCTTTGGATTCTCCAAGCCAGTTTGCTCAAAGTACCCTACTTTGTGCGCGTTCA
AGGCCTTCTCCGGATCTGCGCGCTAGCGCGGAAGATGGCGGGGGGTCATTACGTGCAAATGGTCATCATCAAGCTAGGGGCGCTCACTGGCAC
CTATGTTTATAACCACCTCACTCCTCTTCGGGACTGGGCGCACAACGGCCTGCGAGATCTGGCTGTGGCTGTAGAGCCAGTCGTCTTCTCCCG
AATGGAGACCAACCTCATCACGTGGGGGGCAGACACCGCCGCGTGCGGTGACATCATCAACGGCTTGCCCGTCTCCGCCCGTAGGGGCCGGGA
GATACTGCTCGGACCAGCCGACGGAATGGTCTCCAAGGGGTGGAGGTTGCTGGCACCCATCACGGCATATGCCCAGCAGACGAGGGGCCTCCT
AGGGTGTATAATCACCAGCCTAACCGGCCGGGACAAAAACCAGGTGGAGGGTGAGGTCCAGATTGTGTCAACTGCTGCCCAAACTTTCCTGGC
AACGTGCATCAATGGAGTATGCTGGACCGTCTACCACGGGGCCGGGACGAGGACCATCGCATCACCCAAGGGTCCTGTCATCCAGATGTACAC
CAATGTAGACAAAGATCTTGTGGGCTGGCCCGCTCCCCAAGGTTCCCGTTCATTGACACCCTGCACCTGCGGCTCCTCGGACCTTTATCTGGT
CACGAGGCACGCCGATGTCATTCCCGTGCGCCGGCGGGGTGATAGCAGGGGCAGCCTGCTTTCGCCCCGGCCCATTTCCTACTTGAAAGGCTC
CTCGGGGGGTCCGCTGTTGTGCCCCGCGGGGCATGCTGTGGGCATATTCAGGGCCGCAGTGTGCACCCGTGGAGTGGCTAAGGCGGTGGACTT
TATCCCTGTAGAGAACCTAGAGACAACCATGAGGTCCCCGGTGTTCACGGACAACTCCTCTCCACCGGCAGTACCCCAGAGCTTCCAGGTGGC
CCACCTGCATGCTCCCACAGGCAGCGGCAAGAGCACCAAGGTCCCGGCTGCATACGCAGCTCAGGGCTATAAGGTGCTAGTGCTCAACCCCTC
TGTTGCTGCAACACTAGGCTTCGGTGTTTATATGTCCAAGGCCCATGGGATCGATCCTAACATCAGGACCGGGGTGAGAACAATTACCACTGG
CAGCCCCATCACGTACTCCACCTACGGCAAGTTCCTTGCCGACGGCGGGTGCTCAGGGGGTGCTTATGACATAATAATCTGTGACGAGTGCCA
CTCCACGGATGCCACATCTATCTTAGGCATCGGCACCGTCCTTGACCAAGCAGAGACCGCGGGGGCGAGGCTTGTTGTGCTCGCCACCGCTAC
CCCTCCGGGCTCCGTCACCGTGCCCCATCCTAACATCGAGGAGGTTGCTCTGTCCACCACCGGAGAGATCCCTTTTTATGGCAAGGCTATCCC
CCTCGAAGTAATCAAGGGGGGGAGGCATCTCATCTTCTGTCATTCAAAGAAGAAGTGTGACGAGCTCGCCGCAAAGCTGGTTGCATTGGGCGT
CAATGCCGTGGCCTATTACCGCGGCCTTGACGTGTCTGTCATCCCGACCAGCGGCGATGTTGTCGTCGTGGCAACTGATGCTCTCATGACCGG
CTATACCGGCGACTTCGACTCGGTGATAGACTGCAACACGTGTGTCACCCAGACAGTTGATTTCAGCCTTGACCCCACCTTCACCATTGAGAC
AACCACGCTTCCCCAGGATGCTGTCTCCCGTACACAACGTCGGGGCAGGACTGGCAGAGGGAAGCCAGGCACCTACAGATTTGTGGCACCGGG
GGAGCGCCCCTCCGGCATGTTCGACTCGTCTGTCCTCTGTGAGTGCTATGACGCGGGCTGTGCTTGGTATGAGCTCACGCCCGCCGAGACTAC
AGTTAGGCTACGAGCGTACATGAACACCCCGGGACTTCCCGTGTGCCAGGACCACCTTGAATTTTGGGAGGGCGTCTTTACGGGCCTTACTCA
TATAGATGCCCACTTCCTATCCCAGACAAAGCAGAGCGGGGAGAACCTTCCTTACCTGGTAGCGTACCAAGCCACCGTGTGCGCTAGGGCTCA
AGCCCCTCCCCCATCGTGGGACCAGATGTGGAAGTGCTTGGTCCGCCTCAAGCCCACCCTCCATGGACCAACACCCCTGCTATACAGATTGGG
CGCTGTTCAGAATGAGATCACCCTGACACACCCAATCACAAAATACATCATGACATGTATGTCGGCTGA
Donor 68744/Panel Number 10020/ Transmitted Variant #1 (SEQ ID NO: 16)
TCGCCCACAGGACGTCAAGTTCCCGGGCGGCGGCCAGATCGTTGGTGGAGTTTACTTGTTGCCGCGCAGGGGCCCTAGATTGGGTGTGCGCGC
GACGAGGAAGACTTCCGAGCGGTCGCAACCTCGAGGTAGACGTCAGCCTATCCCCAAGGCGCGTCGGTCCGAGGGCAGGACCTGGGCTCAGCC
CGGGTACCCTTGGCCCCTCTATGGCAATGAGGGTTGCGGATGGGCGGGATGGCTCCTGTCCCCCCGTGGCTCTCGGCCTAGCTGGGGCCCCAC
AGACCCCCGGCGTAGGTCGCGCAATTTGGGCAAGGTCATCGATACCCTCACGTGCGGCTTCGCCGACCTCATGGGGTACATACCGCTCGTCGG
CGCCCCTCTTGGGGGCGCTGCCAGGGCCCTGGCGCATGGAGTCCGGGTTCTGGAAGACGGCGTGAATTATGCAACAGGGAACCTCCCCGGTTG
CTCTTTCTCTATCTTCCTTCTGGCCTTGCTCTCTTGCCTGACTGTGCCCGCTTCAGCCTACCAAGTGCGCAACTCCACGGGGCTTTACCATGT
CACCAATGATTGCCCTAATTCGAGTATTGTGTACGAGGCGGCCGATGCTATCCTGCACACCCCGGGGTGTGTCCCTTGCGTTCGTAAGGGTAA
CACCTCGGTGTGTTGGGTGGCGGTAACCCCTACGGTGGCCACCAGGGACGGCAAACTCCCCACAACGCAGCTTCGACGTCATATCGATCTGCT
CGTCGGGAGTGCCACCCTCTGTTCGGCCCTCTATGTGGGGGACCTGTGCGGGTCTGTCTTTCTTGTTGGCCAACTGTTTACCCTCTCTCCCAG
ACGCCACTGGACAACGCAAGGCTGCAATTGTTCCATCTATCCCGGCCATATAACGGGCCACCGCATGGCATGGGATATGATGATGAACTGGTC
CCCTACAACGGCGTTGGTAATGGCACAGCTGCTCCGGATCCCACAAGCCATCATGGACATGATCGCTGGTGCTCACTGGGGAGTCCTGGCGGG
CATAGCGTATTTCTCTATGGTGGGGAACTGGGCGAAGGTCCTGGTAGTGCTGCTGCTATTTGCCGGCGTCGACGCAAAAACTTATGTCACCGG
GGGAGCTGCTGCCCACACCACGTCCGGACTTGCTGGGCTCTTCTTACAAGGCCCCAAGCAGAACGTCCACTTGATCAACACCAACGGCAGTTG
GCACATCAATAGTACGGCCTTGAACTGCAACGCTAGCCTTGACACCGGCTGGATAGCAGGGCTTTTCTATTACAACAAATTCAACTCTTCAGG
TTGTCCCGAGAGGTTGGCCAGCTGCCGACTCCTTACTGATTTTGACCAGGGCTGGGGCCCTATCAGTCATGCCAACGGAAGCGGCCCCGACCA
ACGCCCCTACTGCTGGCACTACCCCCCAAAACCTTGCGGCATTGTGCCCGCGAAGAGTGTGTGTGGCCCGGTATATTGCTTCACTCCCAGCCC
TGTGGTGGTGGGAACGACCGACAGGTCGGGCGAACCTACCTACAACTGGGGTGAAAATGATACGGACGTCTTCGTCCTTAACAACATCAGGCC
ACCGCTGGGTAATTGGTTTGGTTGTACCTGGATGAACTCAACCGGATTCACCAAAGTGTGCGGGGCGCCTCCTTGTGACATCGGGGGGGCGGG
CAACAACACCTTGCACTGCCCCACTGATTGTTTCCGCAAGCATCCGGAAGCCACGTACTCTCGGTGCGGCTCCGGTCCCTGGATCACGCCCAG
GTGCCTGGTCCACTACCCATACAGGCTTTGGCATTATCCCTGTACCATCAACTACACCATATTTAAAGTCAGGATGTACGTGGGAGGGATCGA
GCACAGGTTGGAAGCTGCCTGCAACTGGACGCGGGGCGAGCGTTGCGATCTGGAAGACAGGGACAGGTCCGAGCTTAGCCCGTTGCTGCTGTC
TACCACACAGTGGCAAGTCCTTCCGTGTTCCTTCACGGCCATGCCAGCCTTGTCCACCGGCCTCATCCACCTCCACCAGAACATTGTGGATGT
GCAGTATTTGTACGGGGTGGGGTCAAGCATTGCGTCCTGGGCCATTAAGTGGGAGTACGTCGTTCTCCTGTTCCTTTTGCTTGCAGACGCGCG
CGTCTGCTCCTGCTTGTGGATGATGTTACTCATATCCCAAGCGGAGGCGGCTTTGGAGAACCTTGTAATACTCAATGCAGCATCCCTGGCCGG
GACGCACGGTCTTGTATCCTTCCTCGTGTTCTTCTGCTTTGCATGGTATCTGAAAGGCAGGTGGGTGCCCGGAGCGGCTTACGCCTTCTACGG
GATGTGGCCTCTCCTCCTGCTCCTGCTGGCGTTGCCCCAGCGGGCATACGCGCTGGACACGGAGGTGGCCGCGTCGTGTGGCGGCGTTGTCCT
TGTCGGGTTAATGGCGCTGACTTTATCACCACACTACAAGCGCTATATCAGCTGGTGCTTATGGTGGCTTCAGTATTTTCTGACCAGAGCGGA
AGCGCAGCTGCACGTGTGGGTTCCCCCCCTCAACGTCCGGGGGGGGCGTGACGCTGTCATCTTACTCATGTGTGTTGTACACCCGACTCTGGT
ATTCGACATCACCAAAATGCTGCTGGCCGTCTATGGACCCCTCTGGATTCTTCAAGCCAGTTTGCTTGAGGTACCCTACTTTGTGCGCGTCCA
AGGCCTTCTCCGGATCTGCGCGCTAGCGCGGAAGATGGTCGGAGGCCATTACGTGCAAATGGCCATCATCAAGTTAGGGGCGCTTACTGGCAC
CTATATTTATAATCATCTCACCCCTCTTCAGGACTGGGCGCACAACGGCCTGCGAGACCTGGCCGCGGCTGTAGAGCCAGTCGTTTTCTCCCA
AATGGAGACCAAGCTCATCACGTGGGGGGCAGACACTGCCGCGTGCGGTGACATTGTCGACGGCTTACCCGTCTCCGCCCGTAGGGGCCGGGA
GATACTGCTTGGGCCGGCCGATGGAATGGTCTCCAAGGGGTGGAGGTTGCTGGCGCCCATCACGGCGTACGCCCAGCAGACAAGGGGCCTCCT
AGGGTGTATAATCACCAGCCTAACTGGCCGGGACAAAAACCAAGTGGAAGGTGAGGTCCAGATTGTGTCGACTGCTGCCCAAACTTTCCTGGC
AACGTGCATCAATGGGGTATGCTGGACTGTCTACCACGGGGCCGGAACGAGGACCATCGCATCACCCAAGGGTCCTGTTATCCAGATGTATAC
CAATGTAGACCAAGACCTTGTGGGCTGGCCCGCTCCTCAAGGTGCCCGCTCATTGACACCCTGCACCTGCGGCTCCTCGGACCTTTACTTGGT
CACGAGGCACGCCGATGTCATTCCCGTGCGCCGGCGGGGTGATAGCAGGGGCAGCCTGCTCTCGCCCCGGCCTATTTCCTACTTGAAAGGCTC
CTCGGGAGGTCCGCTGTTGTGCCCCGCGGGACACGCCGTGGGCATATTTAGGGCCGCGGTGTGTACCCGTGGAGTGGCTAAGGCGGTGGACTT
TATCCCCGTGGAGAGCCTGGAAACAACCATGAGGTCCCCGGTGTTTACGGACAACTCCTCCCCACCAGCGGTGCCCCAGACCTTCCAGGTGGC
CCATTTGCATGCTCCCACAGGCAGCGGCAAGAGCACCAAAGTCCCGGCTGCATATGCAGCTCAGGGTTATAAGGTGCTAGTGCTCAACCCCTC
TGTCGCTGCAACATTGGGCTTTGGTGCCTACATGTCTAAGGCCCATGGGATCGATCCTAACATCAGGACCGGGGTGAGGACAATTACCACTGG
CAGCCCCATCACGTACTCCACCTACGGTAAGTTCCTTGCCGACGGCGGGTGCTCAGGGGGCGCTTATGACATAATAATCTGTGACGAGTGCCA
TTCCACGGATGCCACATCCATCTTGGGCATCGGCACTGTCCTTGACCAAGCAGAGACCGCGGGGGCGAGACTGGTTGTGCTCGCCACCGCTAC
CCCTCCGGGCTCTATCACTGTGCCACATCCCAACATCGAGGAGGTTGCTCTGTCCACCACCGGAGAGATCCCTTTTTACGGCAAGGCCATCCC
CCTTGATGTAATCAAGGGGGGGAGACATCTCATTTTCTGTCACTCAAAGAAGAAGTGTGACGAGCTTGCCGCAAAGCTGGTCGCATTGGGCGT
CAATGCCGTGGCCTACTACCGCGGTCTCGACGTATCTGTCATCCCGACCGGCGGCGATGTTGTCGTCGTGGCAACTGATGCTCTCATGACCGG
CTTTACTGGCGACTTCGACTCGGTGATAGACTGCAACACGTGCGTCACCCAGACAGTCGATTTCAGCCTTGACCCTACCTTCACCATTGAGAC
AACCACGCTTCCCCAGGATGCTGTCTCCCGCACTCAACGTCGGGGCAGGACTGGCAGGGGGAGGCAAGGCACCTACAGATTTGTGGCACCGGG
GGAGCGCCCCTCTGGCATGTTCGACTCGTCTGTCCTCTGTGAGTGCTATGACGCGGGCTGTGCTTGGTACGAGCTCACGCCTGCCGAGACCAC
AGTTAGGCTACGAGCGTATATGAACACCCCGGGGCTTCCCGTGTGTCAAGACCATCTTGAATTTTGGGAGGGCGTCTTTACAGGCCTCACCCA
TATAGATGCCCACTTCCTATCCCAGACAAAACAGAGCGGGGAGAATCTTCCTTACCTGGTAGCGTACCAAGCCACCGTGTGCGCTAGGGCGCA
AGCCCCCCCCCCATCGTGGGACCAGATGTGGAAGTGTTTGGTCCGCCTCAAACCCACCCTCCACGGGCCAACACCCCTGCTATACAGACTGGG
TGCTGTTCAAAATGAAGTCACCCTGACGCACCCAGTCACCAAATACATCATGACATGTATGTCGGCTGA
Donor 65064/ Panel Number 10017/ Transmitted Variant #1 (SEQ ID NO: 17)
ACCAACCGTCGCCCACAGGACGTCAAGTTCCCGGGTGGCGGTCAGATCGTTGGTGGAGTTTACTTGTTGCCGCGCAGGGGCCCTAGATTGGGT
GTGCGCGCGACGAGGAAGACTTCCGAGCGGTCGCAACCTCGTGGTAGACGTCAGCCTATCCCCAAGGCACGTCGGCCCGAGGGCAGGACCTGG
GCTCAGCCCGGGTACCCCTGGCCCCTCTATGGCAATGAGGGCTGCGGGTGGGCGGGATGGCTCCTGTCTCCCCGCGGCTCTCGGCCTAGTTGG
GGCCCCACGGACCCCCGGCGTAGGTCGCGCAATTTGGGTAAGGTCATCGATACCCTCACATGCGGCTTCGCCGACCTCATGGGGTACATACCG
CTCGTCGGCGCCCCTCTTGGAGGCGCTGCCAGGGCCCTGGCGCATGGCGTCCGGGTTCTGGAAGACGGCGTGAACTATGCAACAGGGAATCTT
CCTGGTTGCTCTTTCTCTATCTTCCTTCTGGCTCTGCTCTCTTGCCTGACTGTGCCCGCTTCAGCCTACCAAGTGCGCAACTCTACGGGGCTT
TACCATGTCACCAATGATTGCCCTAACTCGAGCATTGTGTACGAGGCGGCCGATGCCATCCTGCACACTCCGGGGTGTGTCCCTTGCGTCCGC
GAGGGTAACTCCTCGAGGTGTTGGGTGGCGGTGACCCCCACGGTAGCCACTAAGGACGGCAAGCTCCCCACAAGGCAGCTTCGACGTCACATC
GATCTGCTTGTCGGGAGCGCCACCCTCTGCTCGGCCCTCTACGTGGGGGACCTGTGCGGGTCCGTCTTTCTTGTCGGTCAGCTGTTCACCTTC
TCTCCCAGGCGCCACTGGACGACGCAAGACTGCAATTGTTCTATCTATCCCGGCCATATAACGGGTCACCGCATGGCGTGGGATATGATGATG
AACTGGTCCCCTACGGCGGCATTGGTAGTAGCTCAGCTGCTCCGGATCCCACAAGCCATCTTGGACATGATCGCCGGTGCTCACTGGGGAGTC
CTGGCGGGCATGGCGTATTTCTCCATGGTGGGGAACTGGGCGAAGGTCTTGGTAGTGCTGCTGCTATTTGCCGGCGTCGACGCGCAAACCCGC
GTCACCGGGGGAAGTGTCGCCTACACCACGGCTGGTATTGCTAGGTTTTTCCAACCAGGCGCCAAGCAGAACATCCAGCTGATCAACACCAAC
GGCAGTTGGCACATCAATAGCACGGCCTTGAACTGCAATGCAAGCCTTGAAACCGGCTGGTTAGCAGGACTTTTCTATCACAACAGATTCAAC
TCTTCAGGCTGTCCTGAGAGGTTGGCCAGCTGCCGAAGCCTTGCCGACTTTGACCAGGGCTGGGGCCCCATCAGCCATGCCAATGGAAGCGGC
CCCGACCACCGCCCCTACTGCTGGCACTACCCCCCAAAACCTTGTGGTATTGTGCCCGCAAAGAACGTGTGTGGCCCGGTATATTGCTTCACC
CCCAGCCCCGTGGTGGTGGGAACGACCGACAGGGCGGGTGCGCCTACCTACAACTGGGGCGCAAATGAAACGGACGTCTTCGTCCTTAACAAC
ACCAGGCCACCGCAGGGCAATTGGTTCGGTTGTACCTGGATGAACTCAACCGGGTTCACCAAAGTGTGCGGAGCGCCCCCTTGTGCCATCGGA
GGGGTGGGCAACAACACCTTGCACTGCCCCACTGATTGTTTCCGCAAGCATCCGGAAGCCACGTACTCTCGGTGCGGTTCTGGTCCCTGGATC
ACACCCAGGTGCATGGTCCACTACCCGTATAGGCTCTGGCATTATCCCTGTACCATCAACTACACTTTATTTAAAGTCAGGATGTACGTGGGT
GGGGTCGAGCACAGGCTGGAAGCTGCCTGCAACTGGACGCGGGGCGAACGTTGTGATCTGGAAGACAGGGACAGGTCTGAGCTTAGCCCGTTG
CTGCTGTCCACCACACAGTGGCAGGTCCTCCCATGTTCCTTTACGACCCTGCCAGCCTTGTCCACCGGCCTCATCCACCTCCACCAGAACATT
GTGGACGTGCAGTATTTGTACGGGGTGGGGTCAAGCATCGCGTCCTGGGCCATTAAATGGGAGTACGTCGTCCTCCTGTTCCTTCTGCTCGCA
GACGCACGCGTCTGCTCTTGCTTGTGGATGATGTTACTCATATCCCAAGCGGAGGCGGCTTTGGAGAACCTCGTGGTACTCAATGCAGCATCC
CTGGCCGGGACGCACGGTCTTGTACCCTTCCTCGTGTTCTTCTGCTTTGCGTGGTACCTGAAGGGTAGGTGGGTGCCCGGAGCGGTCTACGCC
CTCTACGGGATGTGGCCTCTCCTCCTGCTCTTGTTGGCGTTGCCCCAGCGAGCATACGCACTGGACACGGAGGTGGCCGCGTCGTGTGGCGGC
GTTGTTCTTGTCGGGTTGATGGCGCTGACTCTGTCACCATATTACAAGCACTATATCAGCTGGTGCATGTGGTGGCTTCAGTACTTTCTGACC
AGAGCAGAAGCGCAACTGCACGTGTGGGTTCCCCCTCTCAACGTCCGAGGGGGGCGTGACGCCGTCATCTTACTCACGTGTGTTGTACACCCG
ACTTTGGTATTTGACATCACCAAACTACTCCTGGCCGTTATCGGGCCCCTTTGGATCCTTCAAGCCAGTTTGCTTAAAGTACCCTACTTCGTG
CGCGTTCAGGGCCTTCTCCGAATCTGCGCACTAGCGCGGAAAATGGCCGGAGGCCATTACGTGCAAATGGCCATCATCAAGTTAGGGGCGCTT
ACTGGCACCTATGTCTACAACCATCTCACTCCCCTTCGAGACTGGGCGCACAACGGCCTGCGAGATCTGGCCGTGGCTGTGGAGCCAGTCGTC
TTCTCCCAAATGGAGACCAAGCTCATCACGTGGGGGGCAGACACCGCCGCGTGCGGTGACATCATCGACGGCTTGCCCGTCTCCGCCCGTAGG
GGCCGGGAGATATTGCTCGGGCCGGCCGATGGAATGGTCTCCAAGGGGTGGAGGTTGCTGGCGCCCATCACAGCGTACGCCCAGCAGACAAGG
GGCCTCCTAGGGTGTATAATCACCAGCCTGACTGGCCGGGACAAAAACCAAGCGGAGGGTGAGGTCCAGATTGTGTCAACTGCTGCCCAAACT
TTCCTGGCAACGTGCATCAATGGGGTATGCTGGACTGTCTACCACGGGGCCGGAACGAGGACCATTGCATCACCCAAGGGTCCTGTTATTCAG
ATGTACACCAATGTGGACAAAGACCTCGTGGGCTGGCCCGCCCCTCAAGGTACCCGCTCATTGACACCCTGCACCTGCGGCTCCTCGGACCTT
TACTTGGTCACGAGGCACGCCGATGTCATTCCCGTGCGCCGGCGAGGTGATAGCAGAGGCAGCCTACTTTCGCCCCGGCCCATTTCCTACTTG
AAAGGCTCCTCGGGGGGTCCACTGTTGTGCCCCGCGGGGCACGCCGTGGGCATATTCAGGGCCGCGGTGTGCACCCGTGGAGTGGCTAAAGCG
GTGGACTTTATCCCTGTGGAGAACCTAGAGACAACCATGAGGTCCCCGGTGTTCTCGGACAACTCCTCTCCACCAGCAGTGCCCCAGAGCTTC
CAGGTGGCTCACCTGCATGCTCCCACCGGCAGCGGTAAAAGCACCAAGGTCCCGGCCGCATACGCTGCTCAGGGCTACAAGGTGCTAGTGCTC
AACCCCTCTGTTGCCGCAACACTGGCCTTTGGCGCTTACATGTCCAAGGCCCATGGGGTCGATCCTAATGTCAGGACCGGGGTGAGAACAATT
ACCACTGGCAGCCCAATCACGTACTCCACCTACGGCAAGTTCCTTGCTGACGGCGGGTGCTCGGGGGGTGCCTATGACATAATAATTTGTGAC
GAGTGCCACTCCACGGATGCCACGTCCATCTTGGGCATCGGCACTGTCCTTGACCAAGCAGAGACCGCGGGGGCGAGGCTGGTTGTGCTCGCC
ACCGCTACCCCTCCGGGCTCCGTCACCGTGCCCCATCCTAACATCGAGGAGGTTGCTCTGTCCACCACCGGAGAGGTCCCCTTTTACGGCAAG
GCTATCCCCCTCGAGGTAATCAAGGGGGGGAGACATCTCATCTTCTGTCACTCAAAAAAGAAGTGCGACGAGCTCGCCGCAAAGCTGGTCGCG
TTGGGCATCAATGCCGTGGCCTACTACCGTGGCCTTGACGTGTCTGTCATCCCGACCAGCGGCGATGTTGTCGTCGTGGCGACCGATGCTCTC
ATGACCGGCTATACTGGCGACTTCGACTCGGTGATAGACTGCAACACGTGTGTCACCCAAACAGTCGACTTCAGCCTTGACCCTACCTTCACC
ATTGAGACAACCACGCTCCCCCAGGATGCTGTCTCCCGTACTCAACGCCGGGGCAGGACCGGCAGGGGGAAGTCAGGCATCTACAGATTCGTG
GCACCAGGGGAGCGCCCTTCCGGCATGTTCGACTCGTCCGTCCTCTGTGAGTGCTATGACGCGGGCTGTGCTTGGTATGAGCTCACGCCCGCT
GAGACCACTGTTAGGCTACGAGCGTACATGAACACCCCGGGGCTTCCTGTGTGCCAGGACCATCTTGAGTTTTGGGAGGGCGTTTTTACAGGC
CTCACCCACATAGATGCCCACTTTCTATCCCAGACAAAGCAGAGTGGGGAGAACCTTCCTTACCTGGTAGCGTACCAGGCCACCGTGTGCGCT
AGGGCTCAAGCCCCTCCCCCATCGTGGGACCAGATGTGGAAGTGTTTGATCCGCCTCAAGCCCACCCTCCATGGGCCAACACCTCTGCTATAC
AGACTGGGCGCTGTTCAGAATGAAGTCACCTTGACGCACCCAGTCACCAAATACATCATGACATGCA
Donor 65064/ Panel Number 10017/ Transmitted Variant #2 (SEQ ID NO: 18)
ACCAACCGTCGCCCACAGGACGTCAAGTTCCCGGGTGGCGGTCAGATCGTTGGTGGAGTTTACTTGTTGCCGCGCAGGGGCCCTAGATTGGGT
GTGCGCGCGACGAGGAAGACTTCCGAGCGGTCGCAACCTCGTGGTAGACGTCAGCCTATCCCCAAGGCACGTCGGCCCGAGGGCAGGGCCTGG
GCTCAGCCCGGGTACCCCTGGCCCCTCTATGGCAATGAGGGCTGCGGGTGGGCGGGATGGCTCCTGTCTCCCCGCGGCTCCCGGCCTAGTTGG
GGCCCCACGGACCCCCGGCGTAGGTCGCGCAATTTGGGTAAGGTCATCGATACCCTCACATGCGGCTTCGCCGACCTCATGGGGTACATACCG
CTCGTCGGCGCCCCTCTTGGAGGCGCTGCCAGGGCCCTGGCGCATGGCGTCCGGGTTCTGGAAGACGGCGTGAACTATGCAACAGGGAATCTT
CCTGGTTGCTCTTTCTCTATCTTCCTTCTGGCTCTGCTCTCTTGCCTGACTGTGCCCGCTTCAGCCTACCAAGTGCGCAACTCTACGGGGCTT
TACCATGTCACCAATGATTGCCCTAACTCGAGCATTGTGTACGAGGCGGCCGATGCCATCCTGCACACTCCGGGGTGTGTCCCTTGCGTCCGC
GAGGGTAACTCCTCGAGGTGTTGGGTGGCGGTGACCCCCACGGTGGCCACTAGGGACGGCAAGCTCCCCACAACGCAGCTTCGACGTCACATC
GATCTGCTTGTCGGGAGCGCCACCCTCTGCTCGGCCCTCTACGTGGGGGACCTGTGCGGGTCCGTCTTTCTTGTCGGTCAGCTGTTCACCTTC
TCTCCCAGGCAGCACTGGACGACGCAAGACTGCAATTGTTCTATCTATCCCGGCCATATAACGGGTCACCGCATGGCGTGGGATATGATGATG
AACTGGTCCCCTACGGCGGCATTGGTAGTAGCTCAGCTGCTCCGGATCCCACAAGCCATCTTGGACATGATCGCCGGTGCTCACTGGGGAGTC
CTGGCGGGCATGGCGTATTTCTCCATGGTGGGGAACTGGGCGAAGGTCTTGGTAGTGCTGCTGCTATTTGCCGGCGTCGACGCGTACACCCGC
GTCACTGGGGGAAGTGCCGCCTACACCACGTCTGGTCTTGCTAGGCTGTTCCAACCAGGCGCCCAGCAGAACATCCAGCTGATCAACACCAAC
GGCAGTTGGCACATCAATAGCACGGCCTTGAACTGCAACGCAAGCCTTGACACCGGCTGGTTAGCAGGACTTTTCTATTACAGCAAATTCAAC
TCTTCAGGCTGTCCTGAGAGGTTGGCCAGCTGCCGAAGCCTTGCCGACTTTGACCAGGGCTGGGGCCCCATCAGCCATGCCAATGGAAGCGGC
CCCGACCACCGCCCCTACTGCTGGCACTACCCCCCAAAACCTTGTGGTATTGTGCGCGCAAAGAGCGTGTGTGGCCCGGTATATTGCTTCACC
CCCAGCCCCGTGGTGGTGGGAACGACCGACAGGGCGGGTGCGCCTACCTACAACTGGGGCGCAAATGAAACGGACGTCTTCGTCCTTAACAAC
ACCAGGCCACCGCGGGGCAATTGGTTCGGTTGTACCTGGATGAACTCAACCGGATTCACCAAAGTGTGCGGAGCGCCCCCTTGTGCCATCGGA
GGGGTGGGCAACAACACCTTGCACTGCCCCACTGATTGTTTCCGCAAGCATCCGGAAGCCACGTACTCTCGGTGCGGTTCCGGTCCCTGGATC
ACACCCAGGTGCATGGTCCACTACCCGTATAGGCTCTGGCATTATCCCTGTACCATCAACTACACTTTATTTAAAGTCAGGATGTACGTGGGT
GGGGTCGAGCACAGGCTGGAAGCTGCCTGCAACTGGACGCGGGGCGAACGTTGTGATCTGGAAGACAGGGACAGGTCCGAGCTTAGCCCGTTG
CTGCTGTCCACCACACAGTGGCAGGTCCTCCCATGTTCCTTTACGACCCTGCCAGCCTTGTCCACCGGCCTCATCCACCTCCACCAGAACATT
GTGGACGTGCAGTATTTGTACGGGGTGGGGTCAAGCATCGCGTCCTGGGCCATTAAATGGGAGTACGTCGTCCTCCTGTTCCTTCTGCTCGCA
GACGCACGCGTCTGCTCTTGCTTGTGGATGATGTTACTCATATCCCAAGCGGAGGCGGCCTTGGAGAACCTCGTGGTACTCAATGCAGCATCC
CTGGCCGGGACGCACGGTCTTGTACCCTTCCTCGTGTTCTTCTGCTTTGCGTGGTACCTGAAGGGTAGGTGGGTGCCCGGAGCGGTCTACGCC
CTCTACGGGATGTGGCCTCTCCTCCTGCTCCTGTTGGCGTTGCCCCAGCGGGCATACGCACTGGACACGGAGGTGGCCGCGTCGTGTGGCGGC
GTTGTTCTTGTCGGGTTGATGGCGCTGACTCTGTCACCATATTACAAGCACTATATCAGCTGGTGCATGTGGTGGCTTCAGTACTTTCTGACC
AGAGCAGAAGCGCAACTGCACGTGTGGGTTCCCCCTCTCAACGTTCGAGGGGGGCGTGACGCCGTTATCTTACTCACGTGTGTTGTACACCCG
ACTTTGGTATTTGACATCACCAAACTACTCCTGGCCGTTATCGGGCCCCTTTGGATCCTTCAAGCCAGTTTGCTTAAAGTACCCTACTTCGTG
CGCGTTCAGGGCCTTCTCCGGATCTGCGCACTAGCGCGGAAAATGGCCGGAGGCCATTACGTGCAAATGGCCATCATCAAGTTAGGGGCGCTT
ACTGGCACCTATGTCTACAACCATCTCACTCCCCTTCGAGACTGGGCGCACAACGGCCTGCGAGATCTGGCCGTGGCTGTGGAGCCAGTCGTC
TTCTCCCAAATGGAGACCAAGCTCATCACGTGGGGGGCAGACACCGCCGCGTGCGGTGACATCATCGACGGCTTGCCCGTCTCCGCCCGTAGG
GGCCGGGAGATATTGCTCGGGCCGGCCGATGGAATGGTCTCCAAGGGGTGGAGGTTGCTGGCGCCCATCACAGCGTACGCCCAGCAGACAAGG
GGCCTCCTAGGGTGTATAATCACCAGCCTGACTGGCCGGGACAAAAACCAAGCGGAGGGTGAGGTCCAGATTGTGTCAACTGCTGCCCAAACT
TTCCTGGCAACGTGCATCAATGGGGTATGCTGGACTGTCTACCACGGGGCCGGAACGAGGACCATTGCATCACCCAAGGGTCCTGTTATTCAG
ATGTACACCAATGTGGACAAAGACCTCGTGGGCTGGCCCGCCCCTCAAGGTACCCGCTCATTGACACCCTGCACCTGCGGCTCCTCGGACCTT
TACCTGGTCACGAGGCACGCCGATGTCATTCCCGTGCGCCGGCGAGGTGATAGCAGAGGCAGCCTGCTTTCGCCCCGGCCCATTTCCTACTTG
AAAGGCTCCTCGGGGGGTCCGCTGTTGTGCCCCGCGGGGCACGCCGTGGGCATTTTCAGGGCCGCGGTGTGCACCCGTGGAGTGGCTAAAGCG
GTGGACTTTATCCCTGTGGAGAACCTAGAGACAACCATGAGGTCCCCGGTGTTCTCGGACAACTCCTCTCCACCAGCAGTGCCCCAGAGCTTC
CAGGTGGCTCACCTGCATGCTCCCACCGGCAGCGGTAAAAGCACCAAGGTCCCGGCCGCATACGCTGCTCAGGGCTACAAGGTGCTAGTGCTC
AACCCCTCTGTTGCCGCAACACTGGCCTTTGGCGCTTACATGTCCAAGGCCCATGGGGTCGATCCTAATGTCAGGACCGGGGTGAGAACAATT
ACCACTGGCAGCCCAATCACGTACTCCACCTACGGCAAGTTCCTTGCTGACGGCGGGTGCTCGGGGGGTGCCTATGACATAATAATTTGTGAC
GAGTGCCACTCCACGGATGCCACGTCTATCTTGGGCATCGGTACTGTCCTTGACCAAGCAGAGACCGCGGGGGCGAGGCTGGTTGTGCTCGCC
ACCGCTACCCCTCCGGGCTCCGTCACCGTGCCCCATCCTAACATCGAGGAGGTTGCTCTGTCCACCACCGGAGAGGTCCCCTTTTACGGCAAG
GCTATCCCCCTCGAGGTAATCAAGGGGGGGAGACATCTCATCTTCTGTCACTCAAAAAAGAAGTGCGACGAGCTCGCCGCAAAGCTGGTCGCG
TTGGGCATCAATGCCGTGGCCTACTACCGTGGCCTTGACGTGTCCGTCATCCCGACCAGCGGCGATGTTGTCGTCGTGGCGACCGATGCTCTC
ATGACCGGCTATACTGGCGACTTCGACTCGGTGATAGACTGCAACACGTGTGTCACCCAAACAGTCGACTTCAGCCTTGACCCTACCTTCACC
ATTGAGACAACCACGCTCCCCCAGGATGCTGTCTCCCGTACTCAACGCCGGGGCAGGACCGGCAGGGGGAAGTCAGGCATCTACAGATTCGTG
GCACCGGGGGAGCGCCCTTCCGGCATGTTCGACTCGTCCGTCCTCTGTGAGTGCTATGACGCGGGCTGTGCTTGGTATGAGCTCACGCCCGCT
GAGACCACTGTTAGGCTACGAGCGTACATGAACACCCCGGGGCTTCCTGTGTGCCAGGACCATCTTGAGTTTTGGGAGGGCGTCTTTACAGGC
CTCACCCACATAGATGCCCACTTTCTATCCCAGACAAAGCAGAGTGGGGAGAACCTTCCTTACCTGGTAGCGTACCAGGCCACCGTGTGCGCT
AGGGCTCAAGCCCCTCCCCCATCGTGGGACCAGATGTGGAAGTGTTTGATCCGCCTCAAGCCCACCCTCCATGGGCCAACACCTCTGCTATAC
AGACTGGGCGCTGTTCAGAATGAAGTCACCTTGACGCACCCAGTCACCAAATACATCATGACATGCA
Donor 67787/ Panel Number 10051/ Transmitted Variant #1 (SEQ ID NO: 19)
CCGCCCACAGGACGTCAAGTTCCCGGGCGGTGGTCAGATCGTTGGTGGAGTTTACCTGTTGCCGCGCAGGGGCCCCAGGTTGGGTGTGCGCGC
GACTAGGAAGACTTCCGAGCGGTCGCAACCTCGTGGAAGGCGACAACCTATCCCCAAGGCTCGCCAGCCCGAGGGTAGGGCCTGGGCTCAGCC
TGGGTACCCTTGGCCCCTCTATGGCAATGAGGGCTTGGGGTGGGCAGGATGGCTCCTGTCACCCCGTGGCTCTCGGCCTAGTTGGGGCCCCAC
GGACCCCCGGCGTAGGTCGCGTAATTTGGGTAAGGTCATCGATACCCTTACATGCGGCTTCGCCGACCTCATGGGGTACATTCCGCTCGTCGG
CGCCCCCTTAGGGGGCGCTGCCAGGGCCCTGGCGCATGGCGTCCGGGTTCTGGAGGACGGCGTGAATTATGCAACAGGGAATTTGCCCGGTTG
CTCTTTCTCTATCTTCCTTTTGGCTTTGCTGTCCTGTTTGACCATCCCAGTTTCCGCTTATGAGGTGCGCAACGTGTCCGGGGTGTATCATGT
CACGAACGACTGCTCCAACGCAAGCATTGTGTACGAGGCAACGGACGTGATCATGCATACCCCCGGGTGCGTGCCCTGCGTTCGGGAGGGCAA
TACCTCCCGCTGCTGGGCGGCGCTCACTCCCACACTCGCGGCCAGGAATTCCAGCGTCCCGACTACGACAATACGACGGCACGTCGATTTGCT
CGTTGGGGCGGCTGCTTTCTGTTCCGCTATGTACGTGGGGGATCTCTGCGGATCTGTTTTTCTCGTCTCCCAGCTGTTCACCTTCTCGCCTCG
CCGGCATGAGACGGTACAGGACTGCAATTGCTCAATCTATCCCGGCCACGTATCAGGTCACCGCATGGCTTGGGATATGATGATGAACTGGTC
ACCTACAACAGCCCTAGTGGTATCGCAGCTACTCCGGATCCCACAAGCTGTCGTGGACATGGTGGCGGGGGCCCACTGGGGAGTCCTGGCGGG
CCTTGCCTACTATTCCATGGTGGGGAACTGGGCTAAGGTCTTAATTGTGATGCTACTTTTTGCCGGCGTCGACGGGAGCACCTACACGACAGG
GGGGGCGCTGGGCCGTACCACCCACGGGCTTACGTCCTTCCTTTCAATTGGGCCGTCTCAGAAAATCCAGCTTATAAATACCAACGGCAGCTG
GCACATCAACAGAACTGCCTTGAACTGCAATGAGTCCCTCCACACTGGATGGCTCGCCGCGTTGTTCTACACAAACAAGTTCAACGCGTCCGG
ATGCCCGGAGCGCATGGCCAGCTGCCGCCCCATTGACACGTTCGATCAGGGGTGGGGTCCCATCACTCACGATGGGCAGGGCAGCTCGGACCA
GAGGCCCTACTGTTGGCACTACGCACCCCGACCGTGTGGTATTGTACCCGCGTCGGAGGTGTGTGGTCCAGTGTATTGCTTCACCCCGAGCCC
CGTTGTGGTGGGGACGACCGATCGTTCCGGCGTCCCTACGTATAGCTGGGGGGGGAATGAGACGGACGTGCTGCTCCTCAACAACACGCGGCC
GCCGCAAGGCAACTGGTTCGGCTGTACATGGATGAATAGCACTGGGTTCACCAAAACGTGCGGGGGCCCCCCGTGTAACATCAGGGGGAGCGG
CAATAACACTTTGACCTGCCCCACGGACTGCTTCCGGAAGCACCCCGAGGCCACTTACACCAAATGCGGTTCGGGGCCTTGGTTGACACCTAG
GTGCATGGTTGACTACCCATACAGGCTTTGGCACTACCCCTGCACTGTCAACTTCACCATCTTCAAGGTTAGGATGTATGTGGGGGGCGTGGA
GCACAGGCTCAACGCCGCGTGCAATTGGACTCGAGGAGAGCGTTGTGATTTGGAGGACAGGGATAGATCAGAGCTTAGCCCGCTGCTGCTGTC
TACAACAGAGTGGCAGATACTGCCCTGCTCCTTCACCACCTTACCGGCTCTGTCCACTGGTTTGATCCATCTCCATCAGAACATCGTGGACGT
GCAATACCTGTACGGTATAGGGTCGGCGGTTGTCTCCTTTGCAATCAAGTGGGAGTACATCGTGTTGCTCTTCCTTCTCCTGGCGGACGCGCG
CGTCTGCGCCTGCTTGTGGATGATGCTACTGATAGCTCAGGCCGAGGCTGCCCTAGAGAACCTGGTGGTTCTCAATGCGGCGTCCGTGGCCGG
GGCGCATGGCCTCCTCTCTTTTCTTGTGTTCTTCTGTGCTGCCTGGTACATCAAGGGCAGGTTGGTCCCTGGGGCGGCGTATGCCTTGTACGG
CGTATGGCCGCTACTCCTGCTCCTGTTGGCGTTGCCACCACGGGCATACGCCATGGACCGGGAGATGGCTGCATCGTGCGGAGGCGCGGTCTT
CATAGGTCTGGCACTCTTGACCTTGTCACCACACTATAAAGTGTTTCTCGCCCGGCTCATATGGTGGTTACAATATCTCATCACCAGAGCCGA
GGCGTGTTTGCATGTGTGGATCCCCCCCCTTAACGTTCGGGGGGGCCGCGATGCCATCATCCTCCTCACGTGTATGGTCCACCCAGAGCTAAT
CTTTACCATCACCAAGATCTTGCTCGCCATACTCGGTCCGCTCATGGTGCTCCAGGCTGGTCTAACCAAAGTGCCGTACTTTGTGCGTGCTCA
AGGGCTCATCCGTGTATGCATGTTGGTGCGGAAAGCCGCTGGGGGTCATTACGTCCAAATGGCTTTCATGAGGCTGGCCGCGCTGACGGGTAC
GTACATCTATGACCATCTTACCCCACTGCGGGACTGGGCCCACGCGGGCCTACGAGACCTTGCGGTGGCGGTTGAGCCCGTCGTCTTTTCTGA
CATGGAGACCAAGATCATCACCTGGGGGGCGGACACCGCAGCATGCGGGGACATCATCTCAGGCCTGCCCGTTTCCGCCCGGAGGGGGAGAGA
GATTTTTCTGGGACCGGCCGACGGTCTTGAAGGGCAGGGGTGGCGACTCCTCGCGCCTATCACGGCCTACTCCCAACAGACACGGGGCCTACT
TGGTTGTATCATCACTAGCCTCACAGGCCGGGACAAGAACCAGGTAGAGGGGGAGGTTCAAGTGGTTTCCACTGCAACACAATCTTTCCTGGC
GACCTGCATCAACGGCGTGTGTTGGACTGTCTACCATGGTGCCGGCTCAAAGACCCTAGCCGGCCCTAAGGGCCCAATCACCCAAATGTACAC
CAATGTAGACCAAGACCTCGTTGGCTGGCAAGCGCCCTCTGGGGCGCGCTCCTTGACACCGTGCACCTGCGGCAGCTCGGACCTTTACTTGGT
CACGAGGCATGCTGATGTCATACCGGTGCGCCGGCGGGGCGACAGCAGGGGGAGCTTACTCTCCCCCAGGCCCGTCTCCTACTTGAAGGGTTC
TTCGGGTGGTCCACTGCTTTGCCCCTCGGGGCACGCTGTAGGCATCTTCCGGGCTGCCGTGTGCACCCGGGGGGTTGCGAAGGCGGTGGACTT
CATACCCGTCGAATCTATGGAAACTACCATGCGGTCGCCGGTCTTCACGGACAACTCGTCCCCCCCGGCCGTACCGCAGACATTCCAAGTGGC
CCATCTACACGCCCCCACTGGCAGCGGCAAGAGCACCAAGGTGCCGGCTGCATATGCAGCCCAAGGGTACAAGGTACTTGTCCTGAACCCGTC
CGTCGCCGCCACTTTAGGTTTCGGGGCGTATATGTCCAAGGCACATGGTGTCGACCCTAACATTAGAACTGGGGTAAGGACCATCACCACGGG
CGCCCCTATTACGTACTCCACCTACGGCAAGTTCCTTGCCGACGGTGGTTGTTCTGGAGGCGCCTATGACATCATAATATGTGATGAATGCCA
CTCAACTGACTCGACCAGCATCTTGGGCATCGGCACAGTCCTGGACCAAGCGGAGACGGCTGGAGCGCGGCTTGTCGTGCTCGCCACCGCTAC
GCCTCCGGGATCAGTCACCGTGCCACATCCCAATATCGAGGAGGTGGCTCTGTCCAACATTGGAGAGATCCCCTTCTACGGCAAAGCCATCCC
CATCGAGACCATCAAGGGGGGGAGGCATCTCATTTTCTGCCATTCCAAAAAGAAATGTGACGAACTCGCCGCAAAGCTGACGGGCCTTGGAAT
CAATGCCGTAGCATATTACAGGGGCCTCGATGTGTCCGTCATACCGACCAGCGGAGACGTCGTTGTCGTGGCAACAGACGCTCTAATGACGGG
CTTTACTGGCGATTTTGACTCAGTGATCGACTGTAATACATGTGTCACCCAGACAGTCGATTTCAGCTTGGACCCTACCTTCACCATTGAGAC
CACGACCGTGCCCCAAGACGCGGTGTCGCGCTCACAGCGGCGAGGCAGGACTGGTAGGGGCAGGACAGGCATCTACAGGTTTGTGACTCCAGG
AGAACGGCCTTCGGGCATGTTCGATTCCTCGGTCCTGTGTGAGTGCTATGACGCGGGCTGTGCTTGGTACGAGCTCACACCCGCCGAGACCTC
AGTTAGGCTGCGGGCTTACCTAAATACACCAGGGTTGCCCGTTTGCCAGGACCATCTGGAGTTCTGGGAGAGCGTCTTCACAGGCCTCACCCA
CATAGATGCCCACTTCTTGTCCCAGACTAAACAGGCAGGAGACAACTTTCCCTACCTGGTAGCATACCAGGCTACGGTGTGCGCCAGGGCCCA
GGCTCCACCTCCATCGTGGGATCAAATGTGGAAGTGCCTCATACGGCTAAAGCCTACGCTGCACGGGCCAACACCCCTGCTGTATAGGCTAGG
AGCCGTCCAAAACGAGGTCACCCTCACACACCCCATAACCAAATACATCATGGCATGCATGTCGGCTGA
Donor 61067/ Panel Number 6213/ Transmitted Variant #1 (SEQ ID NO: 20)
CAACCGTCGCCCACAGGACGTTAAGTTCCCGGGTGGCGGTCAGATCGTTGGTGGAGTTTACTTGTTGCCGCGCAGGGGCCCTAGATTGGGTGT
GCGCGCGACGAGGAAGACTTCCGAGCGGTCGCAACCTCGCGGTAGACGTCAGCCTATCCCCAAGGCGCGTCGGCCCGAGGGCAGGACCTGGGC
TCAGCCCGGGTACCCCTGGCCCCTCTACGGCAATGAGGGCTGCGGGTGGGCGGGATGGCTCCTGTCCCCCCGTGGCTCTCGGCCTAGCTGGGG
CCCCACAGACCCCCGGCGTAGGTCGCGCAATTTGGGTAAGGTCATCGACACCCTCACGTGCGGCTTCGCCGACCTCATGGGGTACATTCCGCT
CGTCGGCGCCCCTCTTGGGGGCGCTGCCAGGGCCCTGGCGCATGGCGTCCGGGTCCTGGAAGACGGCGTGAATTATGCAACAGGGAACCTTCC
TGGTTGCTCTTTTTCTATCTTCCTTCTGGCCCTGCTCTCTTGCCTGACCGTGCCCGCGTCGGCCTATCAAGTACGCAACTCCTCGGGCCTCTA
CCATGTCACCAATGATTGCCCTAACTCGAGTATTGTGTACGAGACGGCCGATACCATTCTACATTCTCCGGGGTGTGTCCCTTGCGTTCGCGA
GGGTAACGCCTCGAAATGTTGGGTGGCGGTGGCCCCCACAGTCGCCACCAGGGATGGCAGACTCCCCACAACGCAGCTTCGACGTCACATCGA
TCTGCTTGTCGGGAGCGCCACCCTCTGCTCGGCCCTCTATGTGGGGGACTTGTGCGGGTCTGTCTTCCTTGTCGGTCAACTGTTCACTTTCTC
CCCCAGGCGCCACTGGACAACGCAAGACTGCAACTGTTCTATCTACCCCGGCCATATAACGGGTCACCGCATGGCATGGGATATGATGATGAA
CTGGTCCCCTACAGCAGCGCTGGTAGTAGCTCAGCTGCTCAGGGTCCCGCAAGCCATCTTGGACATGATCGCTGGTGCCCACTGGGGAGTCCT
AGCGGGCATAGCGTATTTCTCCATGGTGGGGAACTGGGCGAAGGTCTTAGTGGTGCTGTTGCTGTTTGCCGGCGTTGATGCGGGAACCCACAT
CACCGGGGGGAGTGTCGCTCATTCCACGGCACGACTCACCGGTCTTTTCAGTCGTGGCTCCCAGCAGCATATCCAGCTGATCAACACCAACGG
CAGTTGGCACATCAATCGCACGGCCCTGAACTGTAATGAAAGCCTCAACACTGGCTGGCTAGCGGGGCTCTTCTATTCCAACAAATTCAACTC
TTCAGGCTGCCCCGAGAGGTTGGCCAGCTGTAGACCCCTTGCCGATTTCGACCAGGGCTGGGGTCCTATCAGCTACGCCAACGGAAGCGGCCC
CGACCACCGGCCCTACTGCTGGCACTACCCCCCAAAGCCTTGTGGTATCGTGCCAGCACAGAGCGTATGTGGCCCAGTGTATTGCTTCACACC
CAGCCCCGTGGTGGTGGGAACGACCAACAAGTTGGGCGCGCCTACCTACAACTGGGGTTGCAATGATACGGACGTCTTCGTCCTTAATAACAC
CAGGCCGCCGTTGGGCAATTGGTTCGGTTGCACCTGGATGAACTCATCTGGATTTACCAAAGTGTGCGGAGCGCCTCCTTGTGTCATCGGAGG
GGGGGGCAACAACACCTTGCACTGCCCCACTGACTGTTTCCGCAAGCATCCGGAAGCCACATACTCTCGGTGTGGCTCCGGTCCCTGGATCAC
GCCCAGGTGCCTGGTCCACTATCCTTATAGGCTTTGGCATTATCCTTGTACTGTCAACTACACCATGTTCAAAGTCAGGATGTACGTGGGAGG
GGTCGAGCACAGGCTGGACGTTGCTTGCAACTGGACGCGGGGCGAGCGTTGCGATCTGGACGACAGGGACAGGTCCGAGCTCAGCCCGCTGCT
GCTGTCCACCACACAGTGGCAGGTCCTTCCGTGTTCCTTTACCACCTTGCCAGCCTTGACTACCGGCCTCATCCACCTCCACCAGAACATCGT
GGATGTGCAATACTTGTACGGGGTGGGGTCAAGCATTGTGTCCTGGGCCATCAAGTGGGAATACGTCATCCTCTTATTTCTCCTGCTTGCAGA
CGCGCGCATCTGCTCCTGCTTGTGGATGATGCTACTCATATCCCAAGCGGAGGCAGCGTTGGAGAACCTTGTGTTGCTCAACGCGGCGTCTCT
GGCCGGGACGCATGGCCTTGCGTCCTTCCTCGTGTTTTTCTGCTTTGCATGGTATCTGAAGGGTAAGTGGGCGCCCGGAGCAGCCTACGCCTT
CTACGGGATGTGGCCTCTCCTCCTGCTCCTGTTGGCGTTGCCCCAGCGGGCATACGCGCTGGACACGGAGATGGCCGCGTCGTGTGGCGGCGT
TGTTATTGTCGGGTTAATGGCGCTGACTCTGTCACCGCACTACAAGCGCTACATCAGCTGGTGCTTATGGTGGCTTCAGTATTTCTTGACCAG
AGTAGAAGCGCAACTGCACGTGTGGGTTCCCCCCCTCAACGTCCGGGGGGGGCGTGACGCTGTCATCCTACTCATGTGTGCTGTACACCCGGC
TTTGGTATTTGACATCACCAAGCTGCTGTTGGCCGTCTTCGGCCCTCTTTGGATTCTTCAAACCAGTCTGCTCAAAGTGCCCTATTTCGTGCG
CGTTCAAGGCCTTCTCCGGATCTGCGCGCTAGCGCGCAAGATGGCCGGCGGCCATTACGTGCAAATGGCCATCATCAAGATGGGGGCGCTTAC
TGGCACCTATGTTTATAACCATCTCACTCCTCTTCGGGACTGGGCGCACAACGGCCTACGAGATCTGGCCGTGGCTGTAGAGCCAGTCGTCTT
CTCCCAGATGGAGACCAAGCTCATCACGTGGGGGGCGGACACCGCCGCGTGCGGTGACATCATCAACGGCTTGCCCGTCTCTGCCCGTAGGGG
CCGAGAGATACTGCTCGGACCGGCCGATGGAATGGTCTCCAAGGGGTGGAGGTTGCTGGCGCCCATCACGGCGTATGCCCAGCAGACAAGGGG
CCTCTTGGGATGCATAATAACCAGCCTGACCGGCCGGGACAAAAACCAGGTGGAGGGTGAGGTTCAGATTGTGTCAACTGCTGCCCAGACCTT
TCTGGCAACCTGCATCAACGGGGTGTGCTGGACTGTCTACCACGGGGCCGGAACAAGGACCATCGCATCACCCAAAGGTCCTGTTATCCAGAT
GTACACCAATGTAGACCAAGACCTCGTAGGCTGGCCCGCTCCCCAAGGTGCCCGCTCATTGACACCCTGCACTTGCGGCTCCTCGGACCTTTA
CCTGGTCACGAGGCACGCCGATGTCATTCCCGTACGCCGACGGGGTGATAGCAGGGGCAGCCTGCTTTCGCCCCGGCCTATCTCTTACTTGAA
AGGCTCCTCGGGGGGTCCACTACTGTGCCCCGCGGGACACGCCGTAGGCATATTCAGGGCCGCGGTGTGCACCCGTGGAGTGGCTAAGGCGGT
GGACTTTATCCCCGTAGAGAGCCTAGAGACAACCATGAGGTCCCCGGTGTTCACAGACAATTCCTCCCCACCAGCAGTGCCCCAGAGCTTCCA
GGTGGCTCACCTGCATGCTCCCACCGGCAGCGGTAAGAGCACCAAGGTCCCGGCCGCATACGCGGCTCAGGGGTACAAGGTGTTGGTGCTCAA
CCCCTCCGTTGCTGCAACACTGGGTTTTGGTGCCTACATGTCCAAGGCCCATGGGGTTGATCCTAACATCAGGACTGGGGTGAGGACAATTAC
TACTGGCAGCTCCATCACGTACTCCACCTACGGCAAGTTCCTCGCCGACGGCGGGTGCTCGGGGGGTGCTTATGACATAATAATTTGTGACGA
GTGCCACTCCACGGATGCAACATCTATCTTGGGCATCGGCACCGTCCTTGACCAAGCAGAGACTGCGGGGGCGAGACTGGTTGTGCTCGCCAC
CGCTACTCCTCCGGGCTCCGTCACCGTGCCCCATCCTAACATCGAGGAGGTTGCTCTGTCTACCACCGGAGAGATCCCCTTTTACGGCAAGGC
TATCCCCCTTGAGGCAATCAAGGGAGGGAGACATCTCATTTTCTGCCACTCAAAGAAGAAGTGCGACGAGCTCGCCGCAAAACTGGTCGCGAT
GGGCATCAATGCCGTGGCTTACTACCGCGGCCTTGACGTGTCCGTCATCCCGACCAGTGGCGACGTTGTCGTTGTGGCAACTGATGCTCTCAT
GACCGGCTATACCGGCGACTTCGACTCGGTGATAGACTGCAACACGTGTGTCACCCAGACAGTCGACTTCAGCCTTGACCCTACCTTCACCAT
TGAGACAACCACACTTCCTCAGGACGCTGTCTCCCGCACCCAACGTCGGGGCAGGACTGGCAGGGGGAAGCCAGGCATCTACAGATTTGTGGC
ACCGGGAGAGCGCCCTTCCGGCATGTTCGACTCGTCCGTCCTCTGTGAGTGCTATGACGCAGGCTGTGCTTGGTATGAGCTCACACCCGCCGA
GACCACAGTCAGGCTACGAGCATACATGAACACTCCGGGGCTTCCCGTGTGCCAAGACCATCTTGAATTTTGGGAGGGCGTCTTCACGGGTCT
CACCCATATAGACGCCCACTTCCTATCTCAGACAAAGCAGAGTGGGGAAAACTTTCCTTACCTGGTAGCGTACCAAGCCACCGTGTGCGCTAG
AGCTCAAGCTCCCCCCCCATCGTGGGACCAGATGTGGAAGTGCTTGATCCGCCTCAAACCCACCCTCCATGGGCCAACACCTCTGCTATACAG
GCTGGGCGCGGTTCAGAATGAAGTCACCCTGACGCACCCAATCACCAAGTACATCATGACATGCATGTCGGCT
Donor 61067/ Panel Number 6213/ Transmitted Variant #2 (SEQ ID NO: 21)
CAACCGTCGCCCACAGGACGTCAAGTTCCCGGGTGGCGGTCAGATCGTTGGTGGAGTTTATTTGTTGCCGCGCAGGGGCCCTAGATTGGGTGT
GCGCGCGACGAGGAAGACTTCCGAGCGGTCGCAACCTCGAGGTAGACGTCAGCCTATCCCCAAGGCGCGTCGGCCCGAGGGCAGGACCTGGGC
TCAGCCCGGGTACCCTTGGCCCCTCTATGGCAATGAGGGCTGCGGGTGGGCGGGATGGCTCCTGTCCCCCCGTGGCTCTCGGCCTAGCTGGGG
CCCCACAGACCCCCGGCGTAGGTCGCGCAATTTGGGTAAGGTCATCGATACCCTCACGTGCGGCTTCGCCGACCTCATGGGGTACATTCCGCT
CGTCGGCGCCCCTCTTGGAGGCGCTGCCAGGGCCCTGGCGCATGGCGTCCGGGTTCTGGAAGACGGCGTGAACTATGCAACAGGGAATCTTCC
CGGTTGCTCTTTCTCTATCTTCCTTCTGGCTCTGCTCTCTTGCCTGACCGTGCCCGCGTCGGCCTACCAAGTACGCAACTCCTCGGGCCTTTA
TCATGTCACCAATGATTGCCCCAACTCGAGTATTGTGTACGAGACGGCCGATACCATCCTACATTCTCCGGGGTGTGTCCCTTGCGTTCGCGA
GGGTAACACCTCAAAATGTTGGGTGCCAGTGTCCCCCACAGTGGCCACCAGGGACGGCAAACTCCCCGCGACGCAGCTTCGACGTCACATCGA
TCTGCTTGTCGGGAGTGCCACCCTCTGTTCAGCCCTATATGTGGGGGACTTGTGCGGGTCTGTCTTTCTTGTCGGTCAACTGTTCACTTTCTC
CCCCAGGCGCCACTGGACAACGCAAGACTGCAACTGCTCTATCTACCCCGGCCATATAACGGGTCACCGCATGGCATGGGATATGATGATGAA
CTGGTCCCCTACAACAGCGCTGGTAGTAGCTCAGCTGCTCAGGGTTCCGCAAGCCATCTTGGATATGATCGCGGGTGCCCACTGGGGGGTCCT
AGCGGGCATAGCGTATTTTTCCATGGTGGGGAACTGGGCGAAGGTCCTGGTAGTGCTGTTGCTGTTTGCCAGCGTCGATGCGGAAACCCGCAC
CACCGGGGGGAGTGCTGCTCACACCACGTTTGGACTCGCCAGTTTATTCAATCCGGGCCCCAGCCAGAAAATCCAGCTGATTAACAGCAACGG
CAGTTGGCACATCAATCGCACAGCCTTGAACTGTAATGCGAGCCTCGACACTGGCTGGGTGGCGGGGCTCCTCTATTACCACAAGTTCAACTC
TTCGGGCTGCACCGAGAGGATGGCCAGCTGTAGACCCCTTGCCAATTTCGCCCAGGGCTGGGGCCCTATCAGCTACGCCAACGGAAGCGGCCC
CGAACACCGCCCCTACTGCTGGCACTACCCTCCAAAACCTTGTGGTATCGTGCCAGCGCTGAATGTATGTGGCCCAGTGTACTGCTTCACTCC
CAGCCCCGTGGTAGTGGGGACGACCGACAAGTTGGGCGCGCCCACCTACAAATGGGGTGCCAATGAGACGGACGTCTTCATTCTTAATAACAC
CAGGCCACCGTTGGGCAATTGGTTTGGTTGCACCTGGATGAATTCATCTGGATTTACCAAAGTGTGCGGAGCGCCTCCTTGTAACATCGGAGG
GATGGGCAACAACACCTTGCACTGCCCCACTGATTGTTTCCGCAAGCATCCGGAAGCCACATACTCTCGGTGTGGCTCCGGCCCCTGGCTCAC
GCCCAGGTGCTTGGTCCACTATGCTTATAGGCTTTGGCATTATCCTTGTACCGTCAACTACACCATGTTCAAAGTCAGGATGTACGTGGGAGG
GGTCGAGCACAGGCTGGACGTGGCCTGCAACTGGACGCGGGGCGAACCTTGTGATCTGGACGACAGGGACAGGTCCGAGCTCAGCCCGCTGCT
GCTGTCCACCACACAGTGGCAGGTCCTTCCGTGCTCCTTCACGACCCTGCCAGCCTTGACTACCGGCCTCATCCACCTCCACCAGAATATCGT
GGACGTGCAATACTTGTACGGGGTGGGGTCGAGCATTGTGTCCTGGGCCATCAAGTGGGAGTACGTCATCCTCTTGTTTCTCCTGCTTGCAGA
CGCGCGCATCTGCTCCTGCTTGTGGATGATGTTGCTCATATCCCAAGCGGAGGCAGCTTTGGAAAATCTTGTATTGCTCAATGCGGCGTCTCT
GGCCGGGACGCACGGTCTTGTGTCCTTCCTCGTGTTTTTCTGCTTTGCATGGTATCTGAAGGGTAGGTGGGTGCCCGGGGTGGCCTACGCCTG
CTACGGGATGTGGCCCTTCCTCCTGCTCCTGTTGGCGTTGCCCCAACGGGCATACGCGCTGGACACGGAGATGGCCGCGTCGTGTGGCGGCAT
TGTTCTTGCCGGGTTAATGGCGCTGACTCTGTCACCGCATTACAAGCGCTATATCAGCTGGTGCTTATGGTGGCTTCAGTATTTCCTGACTAG
AGTAGAAGCGCAACTGCACGTGTGGGTTCCCCCCCTCAACGTTCGAGGGGGGCGCGACGCTGTCATCCTACTCATGTGTGTTGTACACCCGGC
TTTGGTATTTGACATCACCAAGCTACTGCTGGCTGTCTTCGGACCCCTTTGGATTCTTCAGGCCAGTCTGCTCAAAGTGCCCTACTTCGTGCG
CGTTCAAGGCCTTCTCCGGATCTGCGCGCTAGTGCGCAAGGTGGCCGGAGGCCATTACGTGCAGATGGTCATCATCAAGATGGGGGCGCTTGT
TGGCACTTATGTCTATAACCATCTCACTCCTCTTCGGGACTGGGCGCACAACGGCCTGCAAGATCTGGCCGTGGCTGTAGAGCCAGTCGTCTT
CTCCCAAATGGAGACCAAGCTCATCACGTGGGGGGCGGACACCGCCGCGTGCGGTGACATCATCGACGGCTTGCCCGTCTCCGCCCGCAGGGG
CCGAGAGATACTGCTCGGACCGGCTGATGGGATGGTCTCAAAGGGGTGGAGGTTGCTGGCGCCCATCACGGCGTATGCCCAGCAGACAAGGGG
CCTCTTGGGATGCATAATCACCAGCCTGACCGGCCGGGACAAAAACCAGGTGGAGGGTGAGGTTCAAATTGTGTCAACTGCTACCCAGACCTT
CCTGGCGACCTGCATCAACGGGGTGTGCTGGACTGTCTACCACGGGGCCGGAACAAGGACCATCGCGTCGCCCAAGGGCCCTGTCATCCAAAT
GTATACCAATGTGGACCAAGATCTCGTAGGCTGGCCCGCTCCCCAAGGCGCCCGCTCATTGACACCCTGCGCTTGTGGCTCCTCGGACCTTTA
CCTGGTCACGAGGCACGCCGATGTCATTCCTGTGCGCAGGCGGGGTGATAGCAGAGGCAGCCTGCTTTCGCCCCGGCCCATCTCTTACTTGAA
AGGCTCCTCGGGGGGCCCGCTGCTGTGCCCCGCGGGACACGCCGTAGGCATATTCAGAGCTGCGGTGTGCACTCGTGGAGTGGCCAAGGCGGT
GGACTTCATCCCCGTAGAGAGCTTGGAAACAACCATGAGGTCTCCGGTGTTCACAGACAACTCTTCTCCACCAGCAGTGCCCCAGTGCTTCCA
AGTGGCCCACCTACATGCTCCCACCGGCAGCGGTAAGAGTACCAAGGTCCCGGCCGCGTACGCGGCTCAGGGCTACAAGGTGCTGGTGCTCAA
CCCCTCCGTTGCTGCAACGCTGGGCTTTGGTGCTTACATGTCCAAGGCCCATGGGATTGATCCTAACATCAGGACTGGGGTGAGGACAATTAC
TACTGGCAGCCCCATCACGTACTCCACCTACGGCAAGTTTCTTGCCGACGGCGGGTGCTCAGGGGGCGCTTATGACATAATAATTTGTGACGA
GTGCCACTCCACGGATGCAACATCCATCTTGGGCATCGGCACTGTCCTTGACCAAGCAGAGACTGCGGGGGCGAGACTGGTTGTGCTCGCCAC
CGCTACCCCTCCGGGTTCCGTCACCGTGCCCCACCCTAACATCGAGGAGGTTGCTCTGTCCACCACCGGAGAGATTCCTTTTTATGGCAAGGC
TATCCCCCTTGAGACAATCAAGGGGGGGAGACATCTCATTTTTTGCCACTCAAAGAAGAAGTGCGACGAGCTCGCTGCAAAGCTGGTCGCGAT
GGGCGTCAACGCCGTGGCCTACTACCGCGGCCTTGACGTGTCCGTCATCCCGACCAGCGGCGATGTTGTCGTCGTGGCAACTGATGCTCTCAT
GACCGGCTTTACCGGCGACTTCGACTCGGTGATAGATTGCAACACGTGTGTCACCCAGACAGTCGACTTCAGCCTTGATCCTACCTTCACCAT
TGAGACGACCACGCTCCCCCAGGACGCCGTCTCCCGCACTCAACGTCGGGGGAGGACTGGCAGGGGGAAGCCAGGCATCTACAGATTTGTGGC
ACCGGGGGAACGCCCTTCCGGCATGTTCGACTCGTCCGTCCTCTGCGAGTGCTATGACGCGGGCTGTGCTTGGTATGAACTCACACCCGCCGA
GACCACAGTTAGGCTGCGAGCGTACATGAACACCCCGGGGCTCCCCGTGTGCCAAGACCATCTTGAATTTTGGGAGGGCGTCTTTACGGGTCT
CACCCACATAGACGCCCACTTCCTATCTCAGACAAAGCAGAGTGGGGAAAACCTTCCTTACCTGGTAGCGTACCAAGCCACCGTGTGCGCTAG
GGCCCAAGCCCCTCCTCCATCGTGGGACCAAATGTGGAAGTGCCTGATCCGCCTCAAGCCCACCCTTCATGGGCCGACACCTCTGCTATACAG
ACTGGGTGCTGTTCAGAATGAGGTCACCCTGACACACCCAGTCACCAAGTACATCATGACATGCATGTCGGCT
Donor 62286/ Panel Number 6222/ Transmitted Variant #1 (SEQ ID NO: 22)
ACACCAACCGTCGCCCACAGGACGTCAAGTTCCCGGGTGGCGGCCAGATCGTTGGTGGAGTTTACTTGTTGCCGCGCAGGGGCCCTAGATTGG
GTGTGCGCGCGACGAGGAAGACTTCCGAGCGGTCGCAACCTCGAGGTAGACGTCAGCCTATCCCCAAGGCGCGTCGGCCCGAGGGCAGGACCT
GGGCCCAGCCCGGGTACCCTTGGCCCCTCTATGGCAATGAGGGCTGCGGATGGGCGGGATGGCTCCTGTCCCCCCGTGGCTCTCGGCCTAGCT
GGGGCCCCACAGACCCCCGGCGTAGGTCGCGCAATTTGGGTAAGGTCATCGATACCCTCACGTGCGGCTTCGCCGACCTCATGGGGTACATTC
CGCTCGTCGGCGCCCCTCTCGGAGGCGCTGCCAGGGCCCTGGCGCATGGCGTCCGGGTTCTGGAAGACGGCGTGAACTATGCAACAGGGAACC
TTCCTGGTTGCTCTTTCTCTATCTTCCTTCTGGCCCTGCTCTCTTGCCTGACTGTGCCCGCATCAGCCTATCAAGTACGCAACTCTTCGGGCC
TCTACCATGTCACCAATGATTGCCCTAACTCGAGTATTGTGTACGAGACGGCCGATACCATCCTACACTCTCCGGGGTGTGTCCCTTGCGTTC
GCGAGGGTAACGCCTCGAAATGTTGGGTGGCTGTGGCCCCCACGGTCGCCACCAGGGACGGCAAGCTCCCCGCAACGCAGCTTCGACGTCACA
TCGATCTGCTTGTCGGGGGCGCCACCCTCTGCTCGGCCCTCTACGTGGGGGACTTGTGCGGGTCCATCTTTCTTGTCGGCCAACTGTTCACGT
TCTCCCCCAGGCGCCACTGGACAACGCAAGACTGCAACTGTTCTATCTACCCCGGCCACATAACGGGTCATCGCATGGCATGGGATATGATGA
TGAACTGGTCCCCTACAACAGCGCTGGTAGTAGCCCAGCTGCTCAGGGTTCCACAAGCCATCTTGGATATGATCGCTGGTGCTCACTGGGGTG
TCCTAGCGGGCATAGCGTATTTTTCCATGGTGGGGAACTGGGCGAAGGTTCTGGTGGTGCTGCTGCTGTTTGCCGGCGTCGATGCGGCAACCT
ACACCACCGGGGGAGCTGCTGCCAGGACCGCGTCCACGTTCGCCGGCCTTTTCGATACGGGCGCCAAGCAGAATATCCAGCTGATCAACACCA
ACGGCAGTTGGCACATCAATCGCACGGCCTTGAATTGTAATGCGAGCCTCGACACTGGCTGGGTAGCGGGGCTCTTCTATTACCAAAAATTCA
ACTCCTCAGGCTGCCCCGAGAGGATGGCCAGCTGTAAGCCCCTCGCCGATTTCGACCAAGGCTGGGGCCCTATCAGCTACGCCAACGGAAGCG
GCCCCGAACACCGCCCCTACTGCTGGCACTACCCCCCAAAGCCTTGTGGTATCGTGCCAGCGCAGAACGTATGTGGCCCAGTGTATTGCTTCA
CTCCTAGCCCCGTGGTGGTGGGAACAACCGACAAGCGGGGTGCGCCTACCTACAACTGGGGTGGCAATGACACGGACGTCCTCGTCCTTAATA
ACACCAGGCCACCGCTGGGCAATTGGTTCGGTTGCACCTGGATGAACTCATCTGGATTTACCAAAGTGTGCGGAGCGCCTCCTTGTGTCATCG
GAGGAGTGGGCAACAACACCTTGCACTGCCCCACTGACTGTTTCCGCAAGCATCCAGAGGCCACATACTCTCGGTGTGGCTCCGGTCCCTGGA
TCACGCCCAGGTGCATGGTCCACTATCCTTACAGGCTTTGGCATTATCCTTGCACCGTCAACTACACCGTGTTCAAAGTCAGGATGTACGTGG
GAGGGGTCGAGCACAGGCTGGAAGTTGCTTGCAACTGGACGCGGGGCGAGCGTTGTGATCTGGACGACAGGGACAGGTCCGAGCTCAGCCCGC
TACTGCTGTCCACCACACAGTGGCAGGTCCTTCCATGCTCCTTCACGACCTTGCCAGCCTTGACCACCGGCCTCATCCACCTCCACCAGAACA
TCGTGGACGTGCAATATTTGTACGGGGTGGGGTCAAGCATCGTGTCCTGGGCCATCAAGTGGGAGTATGTCATCCTCTTGTTTCTCCTGCTTG
CAGATGCGCGCATCTGCTCCTGCTTGTGGATGATGCTACTCATATCCCAGGCGGAAGCAGCTTTGGAGAACCTCGTGGTGCTCAATGCGGCGT
CTCTGGCCGGGACGCATGGTCTTGTGTCCTTCCTCGTGTTCTTCTGCTTTGCATGGTACCTGAGGGGTAAGTGGGTGCCCGGAGCGGCCTACG
CCTTCTACGGGATGTGGCCTCTCCTCCTGCTCCTGTTGGCGTTGCCCCAGCGGGCATACGCGCTAGACACGGAGACGACCGCGTCGTGTGGCG
GCATTGTTCTTGTCGGGTTAATGGCGCTGACTCTGTCACCGCATTACAAGCGCTATGTCAGCTGGTGCTTATGGTGGCTTCAGTATTTCCTGA
CCAGAGTAGAAGCGCAACTGCACGTGTGGGTCCCCCCCCTCAACGTCCGGGGGGGGCGTGACGCTGTCATCTTACTCACGTGTGTCGTACACC
CGGCCTTGGTATTTGACATCACCAAGCTGCTGCTGGCCGTCTTCGGACCCCTTTGGATTCTTCAAACCAGCCTGCTCAAAGTGCCCTACTTCG
TGCGCGTCCAAGGCCTTCTCCGGATCTGCGCGCTAGTGCGCGAGATGGCCGGAGGCCATTACGTGCAGATGGCCATCATCAAGATGGGGGCGC
TTACTGGCACCTATGTTTATAACCATCTCACCCCTCTTCGGGACTGGGCGCACAACGGCCTACGAGATCTGGCCGTGGCTGTGGAGCCAGTCG
TCTTTTCCCAGATGGAGACCAAGCTCATTACGTGGGGGGCGGACACCGCCGCATGTGGTGACATCATCAACGGCTTGCCCGTCTCCGCCCGTA
GGGGCCGAGAGATATTGCTCGGACCGGCCGACGGGATGGTCTCTAAAGGGTGGAGGTTGCTGGCGCCTATCACGGCGTACGCCCAGCAGACAA
GGGGCCTCTTGGGGTGCATAATTACCAGCCTGACCGGCCGGGACAAAAACCAGGTGGAGGGTGAGGTCCAGATTGTGTCAACTGCTGCCCAGA
CCTTCCTGGCAACCTGCATCAACGGGGTGTGCTGGACTGTCTACCACGGAGCCGGAACAAGGACCATCGCGTCACCCAGGGGTCCTGTTATCC
AGATGTACACCAATGTAGACCAAGACTTAGTAGGCTGGCCCGCTCCCCAAGGTGCCCGCTCATTGACACCCTGCACTTGCGGCTCCTCGGACC
TTTACCTGGTCACGAGGCACGCCGATGTCATTCCCGTGCGCCGGCGGGGTGACAGCAGAGGCAGTCTGCTTTCGCCCCGGCCCATCTCTTACT
TAAAAGGCTCCTCGGGGGGCCCACTGCTGTGCCCCGCGGGACACGCCGTAGGCATATTCAGAGCCGCGGTGTGCACCCGTGGAGTGGCTAAGG
CGGTGGACTTCATCCCCGTAGAGAACCTAGAGACAACCATGAGGTCCCCGGTGTTCACAGACAACTCCTCTCCACCAGCAGTGCCCCAGAGCT
TCCAGGTGGCCCACCTGCATGCTCCCACCGGCAGCGGTAAGAGCACCAAGGTCCCGGCCGCATATGCTGCTCAGGGCTACAAGGTGCTGGTGC
TCAACCCTTCCGTTGCAGCAACATTGGGCTTTGGTGCTTATATGTCCAAGGCCCATGGGATTGATCCTAACATCAGGACTGGGGTGAGGACAA
TTACCACTGGCAGCCCCATCACGTACTCCACCTATGGCAAGTTTCTTGCCGACGGCGGGTGCTCGGGGGGTGCTTATGACATAATAATTTGTG
ACGAGTGCCACTCCACGGATGCAACATCCATCTTGGGCATCGGCACCGTCCTTGACCAAGCAGAGACTGCGGGGGCAAGACTGGTTGTGCTTG
CCACCGCCACCCCTCCGGGCTCCGTCACCGTGCCCCATCATAACATCGAGGAGGTTGCTCTGTCCACCACCGGAGAGATCCCCTTCTATGGCA
AGGCTATTCCCCTTGAGACAATCAAGGGGGGGAGACATCTCATCTTCTGCCATTCAAAGAAGAAGTGCGACGAGCTCGCCGCGAAACTGGCCG
CGTTGGGCGTTAATGCCGTGGCTTACTACCGCGGCCTTGACGTGTCCGTCATCCCGACCAGTGGCGATGTTGTCGTCGTGGCAACTGATGCAC
TCATGACCGGCTATACCGGCGACTTCGACTCGGTGATAGACTGCAATACGTGTGTCACCCAGACAGTCGACTTCAGCCTTGACCCTACCTTCA
CCATTGAGACAACCACGCTTCCCCAGGATGCTGTCTCCCGCACTCAACGTCGGGGCAGGACTGGCAGGGGGAAGCCAGGCATCTACAGATTTG
TGGCACCGGGGGAGCGCCCTTCCGGCATGTTCGACTCGTCCGTCCTCTGTGAGTGCTATGACGCGGGCTGTGCTTGGTATGAGCTCACACCCG
CTGAGACCACAGTTAGGCTACGAGCGTACATGAACACCCCGGGACTCCCTGTGTGCCAAGACCATCTTGAGTTTTGGGAAGGCGTCTTCACGG
GTCTCACCCATATAGACGCCCACTTCTTATCCCAGACAAAGCAGAGTGGAGAAAACTTTCCTTACCTGGTAGCGTACCAAGCCACCGTGTGCG
CTAGAGCCCAAGCCCCTCCCCCATCGTGGGACCAGATGTGGAAGTGTTTGATCCGTCTCAAGCCCACCCTGCATGGGCCAACACCTCTGCTAT
ACAGACTGGGCGCTGTTCAGAATGAAATCACCCTGACGCACCCGGTCACCAAGTATATCATGACATGCATGTCG

We claim:

1. A method for identifying transmitted hepatitis C virus (HCV) genomes, the method comprising:
(a) collecting a patient sample;
(b) isolating viral RNA from said sample;
(c) sequencing said viral RNA, wherein viral RNA sequencing includes HCV genomes of circulating virus;
(d) performing sequence alignment of selected HCV genome regions;
(e) analyzing phylogenetically selected sequence alignments; and
(f) identifying HCV genomes of transmitted virus.

2. The method of claim 2, the method further comprising: (g) detecting the variation in transmitted viral sequence over time, wherein variations in said viral sequences are identified.

3. The method of claim 1, wherein sequencing of HCV genomes is performed by single genome amplification (SGA).

4. The method of claim 1, wherein phylogenetic analysis is performed by mathematical modeling.

5. The method of claim 1, wherein HCV genomes identified according to the method of claim 1 mediate viral transmission.

6. The method of claim 1, wherein the identified HCV genomes of transmitted virus comprise env and core genes.

7. The method of claim 1, wherein HCV genomes comprise global genotypes.

8. The method of claim 7, wherein HCV genomes further comprise subtypes.

9. The method of claim 1, wherein HCV genomes comprise drug resistant variants.

10. The HCV genomes as identified by the method of claim 1, wherein polynucleotide(s) of circulating HCV genomes comprise SEQ ID NOS: 23-757.

11. The HCV genomes as identified by the method of claim 1, wherein said HCV genomes comprise polynucleotides of Table I.

12. An HCV polynucleotide comprising polynucleotides of Table I, wherein said polynucleotides mediate viral transmission.

13. The HCV polynucleotides of claim 12, wherein the polynucleotides comprise env and core genes.

14. A method of administering a vaccine, the method comprising administering one or more HCV polynucleotides of Table I, wherein a patient immune response is induced.

15. The method of claim 14, wherein one or more polypeptides encoded by a polynucleotide(s) of Table I is administered in the presence or absence of additional polynucleotide(s).
